(19) United States
(12) Patent Application Publication
(10) Pub. No.: US 2015/0040249 A1
(43) Pub. Date: Feb. 5, 2015

(54) Non-Human Animal Models of Depression and Methods of Use Thereof

(71) Applicant: The Board of Trustees of the Leland Stanford Junior University, Palo alto, CA (US)

(72) Inventors: Karl A. Deisseroth, Palo Alto, CA (US); Kay M. Tye, Cambridge, MA (US); Melissa R. Warden, San Mateo, CA (US)

(73) Assignee: The Board of Trustees of the Leland Stanford Junior University, Palo Alto, CA (US)

(21) Appl. No.: 14/385,331

(22) PCT Filed: Mar. 13, 2013
(86) PCT No.: PCT/US13/30893

§ 371 Date: Sep. 15, 2014

Related U.S. Application Data
(60) Provisional application No. 61/613,231, filed on Mar. 20, 2012.

(51) Int. Cl. A61K 49/00; C12N 15/85; A01K 67/027

(52) U.S. Cl. CPC A61K 49/0008; A01K 67/0275; C12N 15/8509; A01K 2267/0306; C12N 2015/8536
USPC 800/3; 800/9

(57) ABSTRACT

The disclosure provides non-human optogenetic animal models of depression. Specifically, non-human animals each expresses a light-responsive opsin in a neuron of the animal are provided. The animal models are useful for identifying agents and targets of therapeutic strategies for treatment of depression. Examples of using the non-human animals expressing light-responsive opsin including Halorhodopsin family of light-responsive chloride pumps and Channelrhodopsin family of light-responsive cation channel proteins are described.

### CROSS-REFERENCE

This application claims the benefit of U.S. Provisional Patent Application No. 61/613,231, filed Mar. 20, 2012, which application is incorporated herein by reference in its entirety.

### BACKGROUND

Major depressive disorder is characterized by low mood, suicidal thoughts, reduced motivation, and the inability to experience pleasure. Despite the prevalence of this debilitating psychiatric disease, the most commonly prescribed therapeutic interventions, selective serotonin reuptake inhibitors, are often ineffective and have severe adverse side effects.

Current non-human animal models of depression are non-specific. There is a need in the art for improved non-human animal models of depression.

### SUMMARY

The present disclosure provides non-human optogenetic animal models of depression. The animal models are useful for identifying agents for treating depression, and for identifying targets of therapeutic strategies for treatment of depression.

### BRIEF DESCRIPTION OF THE DRAWINGS

FIGS. 1A-E depict induction of a depression-like phenotype by selective inhibition of the ventral tegmental area (VTA) dopamine (DA) neurons.

FIGS. 2A-E depict rescue of a stress-induced depression-like phenotype by sparse, phasic photoactivation of VTA DA neurons.

FIGS. 3A-C depict the requirement for dopamine, but not glutamine, receptor signaling for mediating escape-related behavior.

FIGS. 4A-I depict modulation of NAc neural encoding of escape-related behavior in the TH::Cre rat by phasic activation of VTA DA neurons.

FIGS. 5A-E depict the use of automated forced swim test (FST) to provide a high temporal resolution readout that can be synchronized with simultaneously recorded neural data.

FIGS. 6A and 6B depict detection of individual kicks in the FST.

FIGS. 7A-C depict use of the magnetic induction method to detect immobility in a cage.

FIGS. 8A-G depict encoding of FST behavioral state by prefrontal neuronal activity.

FIGS. 9A-J depict induction of rapid and reversible behavioral activation in a challenging situation by optogenetic stimulation of mPFC axons in the dorsal raphe nucleus (DRN), but not excitatory medial prefrontal cortex (mPFC).

FIGS. 10A and 10B depict optogenetic stimulation of the rat mPFC.

FIGS. 11A and 11B depict DRN histology and optrode recording.

FIGS. 12A-J depict the effect of optogenetic stimulation of DRN-projecting mPFC neurons on mPFC encoding ability.

FIGS. 13A and 13B depict responses to the conditioned-place aversion test by THcre⁺/eNpHR3.0-eYFP mice and THcre⁺/eNpHR3.0-eYFP mice.

### DEFINITIONS

As used herein, the term “heterologous,” in reference to a nucleic acid, refers to a nucleic acid encoding a gene product (polypeptide or nucleic acid) that is not in its natural environment (i.e., has been altered by the hand of man). For example, a heterologous nucleic acid includes a nucleic acid from one species introduced into another species. A heterologous nucleic acid also includes a gene native to an organism that has been altered in some way (e.g., mutated, added in multiple copies, linked to a non-native promoter or enhancer sequence, etc.). Heterologous nucleic acids may comprise a nucleotide sequence that comprises cDNA forms of the nucleic acid; the cDNA sequences may be expressed in either a sense (to produce mRNA) or anti-sense orientation (to produce an anti-sense RNA transcript that is complementary to the mRNA transcript). Heterologous nucleic acids can in some embodiments distinguished from endogenous nucleic acids in that the heterologous nucleic acid sequences are typically joined to nucleotide sequences comprising regulatory elements such as promoters that are not found naturally associated with the gene for the protein encoded by the heterologous gene or with gene sequences in the chromosome, or are associated with portions of the chromosome not found in nature (e.g., genes expressed in loci where the gene is not normally expressed).

As used herein, the term “non-human mammal” refers to any non-human mammal, including, but not limited to, non-human primates, rodents (e.g., mice, rats, etc.), and the like. In some cases, the non-human mammal is a mouse. In other cases, the non-human mammal is a rat.

As used herein, “mood disorder” refers to disruption of feeling tone or emotional state experienced by an individual for an extensive period of time. Mood disorders include, but are not limited to, major depression disorder (i.e., unipolar disorder), mania, dysphoria, bipolar disorder, dysthymia, cyclothymia and the like. See, e.g., Diagnostic and Statistical Manual of Mental Disorders, Fourth Edition, (DSM IV).

As used herein, “anxiety disorder” refers to unpleasant emotional state comprising psychophysiological responses to anticipation of unreal or imagined danger, ostensibly resulting from unrecognized intrapsychic conflict. Physiological concomitants include increased heart rate, altered respiration rate, sweating, trembling, weakness, and fatigue; psychological concomitants include feelings of impending danger, powerlessness, apprehension, and tension. Anxiety disorders include, but are not limited to, panic disorder, obsessive-compulsive disorder, post-traumatic stress disorder, social phobia, social anxiety disorder, specific phobias, generalized anxiety disorder.

“Obsessive compulsive disorder” or “OCD” is an anxiety disorder characterized by recurrent obsessions or compulsions sufficient to cause marked distress in the individual. They are typically time-consuming, and/or significantly interfere with the person's normal functioning, social activities, or relationships. Obsessions are recurrent ideas, thoughts, images, or impulses that enter the mind and are persistent, intrusive, and unwelcome. Often, attempts are made to ignore or suppress the thoughts, or to neutralize them with some other thought or action. The individual may recognize the obsessions as a product of his or her own mind. Compulsions are repetitive, purposeful behaviors or movements performed in response to an obsession, and are typically designed to neutralize or prevent discomfort or some dreaded event or situation. For example, a common obsession concerns thoughts of contamination; excessive, repetitive, and non-purposeful hand washing is a common compulsion.

“Major depression disorder,” “major depressive disorder,” or “unipolar disorder” refers to a mood disorder involving any of the following symptoms: persistent sad, anxious, or “empty” mood; feelings of hopelessness or pessimism; feelings of guilt, worthlessness, or helplessness; loss of interest or pleasure in hobbies and activities that were once enjoyed, including sex; decreased energy, fatigue, being “slowed down”; difficulty concentrating, remembering, or making decisions; insomnia, early-morning awakening, or oversleeping; appetite and/or weight loss or overeating and weight gain; thoughts of death or suicide or suicide attempts; restlessness or irritability, or persistent physical symptoms that do not respond to treatment, such as headaches digestive disorders, and chronic pain. Various subtypes of depression are described in, e.g., DSM IV.

“Bipolar disorder” is a mood disorder characterized by alternating periods of extreme moods. A person with bipolar disorder experiences cycling of moods that usually swing try being overly elated or irritable (mania) to sad and hopeless (depression) and then back again, with periods of normal mood in between. Diagnosis of bipolar disorder is described in, e.g., DSM IV. Bipolar disorders include bipolar disorder I (mania with or without major depression) and bipolar disorder II (hypomania with major depression), see, e.g., DSM IV.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “a light-activated cation channel” includes a plurality of such light-activated cation channels and reference to “the depressive behavior” includes reference to one or more depressive behaviors and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION

The present disclosure provides non-human optogenetic animal models of depression. The animal models are useful for identifying agents for treating depression, and for identifying targets of therapeutic strategies for treatment of depression.

### Non-Human Animal Models of Depression

The present disclosure provides a non-human animal that expresses a light-responsive opsin (e.g., a light-responsive ion channel; a light-responsive ion pump; etc.) in a neuron of the animal. Activation of the light-responsive opsin by exposure of the light-activated opsin to light modulates the behavior of the animal. In particular embodiments, light activation of the light-responsive opsin induces depression in the animal. In other embodiments, light activation of the light-responsive opsin relieves depression.

In some cases, a subject non-human animal model of depression exhibits symptoms of depression in the presence of light that activates the light-responsive opsin. In other cases, a subject non-human animal model of depression exhibits symptoms of depression in the absence of light that activates the light-responsive opsin.

A subject non-human animal model of depression can be used to analyze the effect of a test agent on any of a variety of adverse psychological and physiological states, including, but not limited to, dysphoria, depression, anhedonia, suicidality, agitation, anxiety, drug addiction withdrawal symptoms, and the like. In some cases, a test agent that reduces or alleviates an adverse state is considered a candidate agent for treating a mood disorder (e.g., major depression disorder (i.e., unipolar disorder), mania, dysphoria, bipolar disorder, dysthymia, cyclothymia, and the like). Thus, although depression is discussed, a subject screening method can be used to analyze the effect of a test agent on any of a variety of adverse states; and test agents identified can be considered candidate agents for treating any of a variety of mood disorders and other adverse psychological and physiological states.

Symptoms of depression in the non-human animal model include, e.g., reduced escape-related behavior, anxiety, and stress. Tests for depression and/or anxiety and/or stress include the forced swim test (FST) (see, e.g., Porsolt et al. (1977) Nature 266:730; and Petit-Demouliere, et al. (2005) Psychopharmacology 177: 245); the tail suspension test (see, e.g., Cryan et al. (2005) Neurosci. Behav. Rev. 29:571; and Li et al. (2001) Neuropharmacol. 40:1028); conditioned place aversion (see, e.g., Bechtholt-Gompf et al. (2010) Neuropsychopharmacol. 35:2049); the novelty hypophagia test (Dulawa, et al. (2005) Neurosci. Biobehav. Rev. 29:771); the social defeat stress test (see, e.g., Blanchard et al. (2001) Physiol Behav. 73:261-271; and Kudryavtseva et al. (1991) Pharmacol. Biochem. Behav. 38: 315); the sucrose preference test (see, e.g., Kurre Nielsen, et al. (2000) Behavioural Brain Research 107:21-33); the open field test (see, e.g., Holmes (2001) Neurosci. Biobehav. Rev. 25:261-273); the elevated plus maze test (see, e.g., Holmes (2001) supra); and the like.

A nucleic acid comprising a nucleotide sequence encoding a light-responsive opsin is introduced into a non-human mammal. A nucleic acid comprising a nucleotide sequence encoding a light-responsive opsin is also referred to herein as a “heterologous nucleic acid” or a “transgene.” The nucleic acid is expressed, such that the light-responsive opsin is synthesized in a neuron in the non-human mammal.

The light-responsive opsin can, when exposed to light at an activating wavelength (a wavelength that activates the opsin), either promoter hyperpolarization or depolarization of the plasma membrane of a cell (e.g., a neuron) in which the light-responsive opsin is expressed. For example, where a light-activated opsin is expressed in a dopaminergic (DA) neuron of the ventral tegmental area, and where the light-activated opsin promotes hyperpolarization in the presence of light of an activating wavelength, the activity of the DA neurons is inhibited. As another example, where a light-activated opsin is expressed in a DA neuron of the ventral tegmental area, and where the light-activated opsin promotes depolarization of the neurons when activated by light of an activating wavelength, the DA neuron is activated. As another example, where a light-activated opsin is expressed in an excitatory (glutamaergic) neuron in the medial prefrontal cortex, and where the light-activated opsin promotes depolarization of the neurons when activated by light of an activating wavelength, the excitatory neurons are activated.

In some cases, the transgene is integrated into the genome of a neuron in the non-human mammal. Integration into the genome can be targeted, e.g., the transgene is integrated at a specific, targeted site in the genome. Integration into the genome of the neuron can be non-targeted, e.g., the transgene integrates into the genome at a random site. In other cases, the transgene remains episomal, e.g., the transgene is not integrated into the genome of the non-human mammal. In some cases, the transgene is present in substantially all cells of the mammal; in other cases, the transgene is present in only a subset of the cells of the mammal (e.g., the transgene is present only in a neuronal cell population in the mammal). Where the transgene is present in substantially all cells of the mammal, in many embodiments the transgene is expressed in only a subset of the cells, e.g., only in a neuronal cell population of the mammal.

Introduction of a Transgene into a Subset of Cells

As noted above, in some cases, a transgene (e.g., nucleic acid comprising a nucleotide sequence encoding a light-responsive opsin) is present in only a subset of cells of a mammal. For example, in some cases, the transgene is present only in brain cells. In some of these embodiments, the transgene is integrated into the genome (either at a random integration site, or at a targeted integration site) of the subset of cells. In other cases, the transgene remains episomal.

In some cases, the light-responsive opsin-encoding nucleotide sequence is operably linked to one or more transcriptional control elements that provide for cell type-specific expression of the transgene. For example, in some cases, the light-responsive opsin-encoding nucleotide sequence is operably linked to a control element (e.g., a promoter) that provides for neuron-specific expression of the transgene. In some cases, the neuron-specific promoter provides for expression of the transgene in a sub-type of neurons, e.g., dopaminergic neurons, excitatory neurons, neurons of the medial prefrontal cortex, and the like.

Neuron-specific promoters and other control elements (e.g., enhancers) are known in the art. Suitable neuron-specific control sequences include, but are not limited to, a neuron-specific enolase (NSE) promoter (see, e.g., EMBL HSENO2, X51956); an aromatic amino acid decarboxylase (AADC) promoter; a neurofilament promoter (see, e.g., GenBank HUMNFL, L04147); a synapsin promoter (see, e.g., GenBank HUMSYNIB, M55301); a thy-1 promoter (see, e.g., Chen et al. (1987) Cell 51:7-19; and Llewellyn, et al. (2010) Nat. Med. 16(10):1161-1166); a serotonin receptor promoter (see, e.g., GenBank S62283); a tyrosine hydroxylase promoter (TH) (see, e.g., Oh et al. (2009) Gene Ther 16:437; Sasaoka et al. (1992) Mol. Brain Res. 16:274; Boundy et al. (1998) J. Neurosci. 18:9989; and Kaneda et al. (1991) Neuron 6:583-594); a GnRH promoter (see, e.g., Radovick et al. (1991) Proc. Natl. Acad. Sci. USA 88:3402-3406); an L7 promoter (see, e.g., Oberdick et al. (1990) Science 248:223-226); a DNMT promoter (see, e.g., Bartge et al. (1988) Proc. Natl. Acad. Sci. USA 85:3648-3652); an enkephalin promoter (see, e.g., Comb et al. (1988) EMBO J. 17:3793-3805); a myelin basic protein (MBP) promoter; a Ca²⁺-calmodulin-dependent protein kinase II-alpha (CamKIIα) promoter (see, e.g., Mayford et al. (1996) Proc. Natl. Acad. Sci. USA 93:13250; and Casanova et al. (2001) Genesis 31:37); and a CMV enhancer/platelet-derived growth factor-β promoter (see, e.g., Liu et al. (2004) Gene Therapy 11:52-60).

A transgene (e.g., nucleic acid comprising a nucleotide sequence encoding a light-responsive opsin) can be injected directly into a tissue of interest, or adjacent to a tissue of interest, to provide for expression of the light-responsive opsin in the tissue of interest. For example, the transgene can be injected into, or adjacent to, a brain region of interest, e.g., the transgene can be injected into, or adjacent to, the prefrontal cortex, the ventral tegmental area, etc.

Integration into the Genome of a Zygote or ES Cell

In another aspect, the present disclosure provides a zygote or embryonic stem (ES) cell whose genome comprises a transgene (e.g., nucleic acid comprising a nucleotide sequence encoding a light-responsive opsin). A DNA construct which comprises the transgene may be integrated into the genome of the transgenic mammal by any standard method such as those described in Hogan et al., “Manipulating the Mouse Embryo”, Cold Spring Harbor Laboratory Press, 1986; Kraemer et al., “Genetic Manipulation of the Early Mammalian Embryo”, Cold Spring harbor Laboratory Press, 1985; Wagner et al., U.S. Pat. No. 4,873,191, Krimpenfort et al U.S. Pat. No. 5,175,384 and Krimpenfort et al., Biotechnology, 9: 88 (1991), all of which are incorporated herein by reference. As an example, a transgene is microinjected into pronuclei of zygotes of non-human mammalian mammals, such as mice, rats, etc. These injected embryos are transplanted to the oviduts or uteri of pseudopregnant females from which founder mammals are obtained. The founder mammals (Fo), are transgenic (heterozygous) and can be mated with non-transgenic mammals of the same species to obtain F1 non-transgenic and transgenic offspring at a ratio of 1:1. A heterozygote mammal from one line of transgenic mammals may be crossed with a heterozygote mammal from a different line of transgenic mammals to produce mammals that are heterozygous at two loci. Mammals whose genome comprises the transgene are identified by standard techniques such as polymerase chain reaction, Southern blot assays, or other methods known in the art.

In some cases, the light-responsive opsin-encoding nucleotide sequence is operably linked to one or more transcriptional control elements that provide for cell type-specific expression of the transgene. For example, in some cases, the light-responsive opsin-encoding nucleotide sequence is operably linked to a control element (e.g., a promoter) that provides for neuron-specific expression of the transgene. In some cases, the neuron-specific promoter provides for expression of the transgene in a sub-type of neurons, e.g., dopaminergic neurons, excitatory neurons, neurons of the medial prefrontal cortex, and the like. Exemplary promoters include those listed above.

### Light-Responsive Opsins

Optogenetics refers to the combination of genetic and optical methods used to control specific events in targeted cells of living tissue, even within freely moving mammals and other animals, with the temporal precision (millisecond-timescale) needed to keep pace with functioning intact biological systems. Optogenetics requires the introduction of fast light-responsive channel or pump proteins to the plasma membranes of target neuronal cells that allow temporally precise manipulation of neuronal membrane potential while maintaining cell-type resolution through the use of specific targeting mechanisms. Any microbial opsin that can be used to promote neural cell membrane hyperpolarization or depolarization in response to light may be used. For example, the Halorhodopsin family of light-responsive chloride pumps (e.g., NpHR, NpHR2.0, NpHR3.0, NpHR3.1) and the GtR3 proton pump can be used to promote neural cell membrane hyperpolarization in response to light. Additionally, members of the Channelrhodopsin family of light-responsive cation channel proteins (e.g., ChR2, SFOs, SSFOs, C1V1s) can be used to promote neural cell membrane depolarization or depolarization-induced synaptic depletion in response to a light stimulus.

### Light-Responsive Chloride Pumps

In some cases, a light-responsive opsin expressed in a neural cell of a non-human animal model is a light-responsive ion pump, e.g., a light-responsive chloride pump. For example, one or more members of the Halorhodopsin family of light-responsive chloride pumps are expressed on the plasma membranes of neural cells. In some embodiments, one or more light-responsive chloride pumps are expressed on the plasma membrane of a neuron in the VTA. In other embodiments, one or more light-responsive chloride pumps are expressed on the plasma membrane of a neuron in the mPFC.

In some aspects, said one or more light-responsive chloride pump proteins expressed on the plasma membranes of a neuron described above can be derived from Natronomonas pharaonis. In some embodiments, the light-responsive chloride pump proteins can be responsive to amber light as well as red light and can mediate a hyperpolarizing current in the nerve cell when the light-responsive chloride pump proteins are illuminated with amber or red light. The wavelength of light which can activate the light-responsive chloride pumps can be between about 580 and 630 nm. In some embodiments, the light can be at a wavelength of about 589 nm or the light can have a wavelength greater than about 630 nm (e.g. less than about 740 nm). In another embodiment, the light has a wavelength of around 630 nm. In some embodiments, the light-responsive chloride pump protein can hyperpolarize a neural membrane for at least about 90 minutes when exposed to a continuous pulse of light. In some embodiments, the light-responsive chloride pump protein can comprise an amino acid sequence at least about 90%, 91%, 92%, 93%, 94%, 95%, 96%, 97%, 98%, 99%, or 100% identical to the sequence shown in SEQ ID NO: 1. Additionally, the light-responsive chloride pump protein can comprise substitutions, deletions, and/or insertions introduced into a native amino acid sequence to increase or decrease sensitivity to light, increase or decrease sensitivity to particular wavelengths of light, and/or increase or decrease the ability of the light-responsive protein to regulate the polarization state of the plasma membrane of the cell. In some embodiments, the light-responsive chloride pump protein contains one or more conservative amino acid substitutions. In some embodiments, the light-responsive protein contains one or more non-conservative amino acid substitutions. The light-responsive protein comprising substitutions, deletions, and/or insertions introduced into the native amino acid sequence suitably retains the ability to hyperpolarize the plasma membrane of a neuronal cell in response to light.

Additionally, in other aspects, the light-responsive chloride pump protein can comprise a core amino acid sequence at least about 90%, 91%, 92%, 93%, 94%, 95%, 96%, 97%, 98%, 99%, or 100% identical to the sequence shown in SEQ ID NO: 1 and an endoplasmic reticulum (ER) export signal. This ER export signal can be fused to the C-terminus of the core amino acid sequence or can be fused to the N-terminus of the core amino acid sequence. In some embodiments, the ER export signal is linked to the core amino acid sequence by a linker. The linker can comprise any of about 5, 10, 20, 30, 40, 50, 75, 100, 125, 150, 175, 200, 225, 250, 275, 300, 400, or 500 amino acids in length. The linker may further comprise a fluorescent protein, for example, but not limited to, a yellow fluorescent protein, a red fluorescent protein, a green fluorescent protein, or a cyan fluorescent protein. In some embodiments, the ER export signal can comprise the amino acid sequence FXYENE (SEQ ID NO:12), where X can be any amino acid. In another embodiment, the ER export signal can comprise the amino acid sequence VXXSL, where X can be any amino acid. In some embodiments, the ER export signal can comprise the amino acid sequence FCYENEV (SEQ ID NO:13).

Endoplasmic reticulum (ER) export sequences that are suitable for use in a modified opsin include, e.g., VXXSL (where X is any amino acid) (e.g., VKESL (SEQ ID NO:14); VLGSL (SEQ ID NO:15); etc.); NANSFCYENEVALTSK (SEQ ID NO:16); FXYENE (SEQ ID NO:12; where X is any amino acid), e.g., FCYENEV (SEQ ID NO:13); and the like. An ER export sequence can have a length of from about 5 amino acids to about 25 amino acids, e.g., from about 5 amino acids to about 10 amino acids, from about 10 amino acids to about 15 amino acids, from about 15 amino acids to about 20 amino acids, or from about 20 amino acids to about 25 amino acids.

In other aspects, the light-responsive chloride pump protein expressed in a neuron in a non-human animal model of the present disclosure can comprise a light-responsive protein expressed on the cell membrane, wherein the protein comprises a core amino acid sequence at least about 90%, 91%, 92%, 93%, 94%, 95%, 96%, 97%, 98%, 99%, or 100% identical to the sequence shown in SEQ ID NO: 1 and a trafficking signal (e.g., which can enhance transport of the light-responsive chloride pump protein to the plasma membrane). The trafficking signal may be fused to the C-terminus of the core amino acid sequence or may be fused to the N-terminus of the core amino acid sequence. In some embodiments, the trafficking signal can be linked to the core amino acid sequence by a linker, which can comprise any of about 5, 10, 20, 30, 40, 50, 75, 100, 125, 150, 175, 200, 225, 250, 275, 300, 400, or 500 amino acids in length. The linker may further comprise a fluorescent protein, for example, but not limited to, a yellow fluorescent protein, a red fluorescent protein, a green fluorescent protein, or a cyan fluorescent protein. In some embodiments, the trafficking signal can be derived from the amino acid sequence of the human inward rectifier potassium channel Kir2.1. In other embodiments, the trafficking signal can comprise the amino acid sequence KSRITSEGEYIPLDQIDINV (SEQ ID NO:17).

In some aspects, the light-responsive chloride pump protein can comprise a core amino acid sequence at least about 90%, 91%, 92%, 93%, 94%, 95%, 96%, 97%, 98%, 99%, or 100% identical to the sequence shown in SEQ ID NO: 1 and at least one (such as one, two, three, or more) amino acid sequence motifs which enhance transport to the plasma membranes of mammalian cells selected from the group consisting of an ER export signal, a signal peptide, and a membrane trafficking signal. In some embodiments, the light-responsive chloride pump protein comprises an N-terminal signal peptide, a C-terminal ER Export signal, and a C-terminal trafficking signal. In some embodiments, the C-terminal ER Export signal and the C-terminal trafficking signal can be linked by a linker. The linker can comprise any of about 5, 10, 20, 30, 40, 50, 75, 100, 125, 150, 175, 200, 225, 250, 275, 300, 400, or 500 amino acids in length. The linker can also further comprise a fluorescent protein, for example, but not limited to, a yellow fluorescent protein, a red fluorescent protein, a green fluorescent protein, or a cyan fluorescent protein. In some embodiments the ER Export signal can be more C-terminally located than the trafficking signal. In other embodiments the trafficking signal is more C-terminally located than the ER Export signal. In some embodiments, the signal peptide comprises the amino acid sequence MTETLPPVTESAVALQAE (SEQ ID NO:18). In another embodiment, the light-responsive chloride pump protein comprises an amino acid sequence at least 95% identical to SEQ ID NO:2.

Moreover, in other aspects, the light-responsive chloride pump proteins can comprise a core amino acid sequence at least about 90%, 91%, 92%, 93%, 94%, 95%, 96%, 97%, 98%, 99%, or 100% identical to the sequence shown in SEQ ID NO: 1, wherein the N-terminal signal peptide of SEQ ID NO:1 is deleted or substituted. In some embodiments, other signal peptides (such as signal peptides from other opsins) can be used. The light-responsive protein can further comprise an ER transport signal and/or a membrane trafficking signal described herein. In some embodiments, the light-responsive chloride pump protein comprises an amino acid sequence at least 95% identical to SEQ ID NO:3.

In some embodiments, the light-responsive opsin protein is a NpHR opsin protein comprising an amino acid sequence at least 95%, at least 96%, at least 97%, at least 98%, at least 99% or 100% identical to the sequence shown in SEQ ID NO:1. In some embodiments, the NpHR opsin protein further comprises an endoplasmic reticulum (ER) export signal and/or a membrane trafficking signal. For example, the NpHR opsin protein comprises an amino acid sequence at least 95% identical to the sequence shown in SEQ ID NO:1 and an endoplasmic reticulum (ER) export signal. In some embodiments, the amino acid sequence at least 95% identical to the sequence shown in SEQ ID NO:1 is linked to the ER export signal through a linker. In some embodiments, the ER export signal comprises the amino acid sequence FXYENE (SEQ ID NO:12), where X can be any amino acid. In another embodiment, the ER export signal comprises the amino acid sequence VXXSL, where X can be any amino acid. In some embodiments, the ER export signal comprises the amino acid sequence FCYENEV (SEQ ID NO:13). In some embodiments, the NpHR opsin protein comprises an amino acid sequence at least 95% identical to the sequence shown in SEQ ID NO:1, an ER export signal, and a membrane trafficking signal. In other embodiments, the NpHR opsin protein comprises, from the N-terminus to the C-terminus, the amino acid sequence at least 95% identical to the sequence shown in SEQ ID NO:1, the ER export signal, and the membrane trafficking signal. In other embodiments, the NpHR opsin protein comprises, from the N-terminus to the C-terminus, the amino acid sequence at least 95% identical to the sequence shown in SEQ ID NO:1, the membrane trafficking signal, and the ER export signal. In some embodiments, the membrane trafficking signal is derived from the amino acid sequence of the human inward rectifier potassium channel Kir2.1. In some embodiments, the membrane trafficking signal comprises the amino acid sequence K S R I T S E G E Y I P L D Q I D I N V (SEQ ID NO:17). In some embodiments, the membrane trafficking signal is linked to the amino acid sequence at least 95% identical to the sequence shown in SEQ ID NO:1 by a linker. In some embodiments, the membrane trafficking signal is linked to the ER export signal through a linker. The linker may comprise any of 5, 10, 20, 30, 40, 50, 75, 100, 125, 150, 175, 200, 225, 250, 275, 300, 400, or 500 amino acids in length. The linker may further comprise a fluorescent protein, for example, but not limited to, a yellow fluorescent protein, a red fluorescent protein, a green fluorescent protein, or a cyan fluorescent protein. In some embodiments, the light-responsive opsin protein further comprises an N-terminal signal peptide. In some embodiments, the light-responsive opsin protein comprises the amino acid sequence of SEQ ID NO:2. In some embodiments, the light-responsive opsin protein comprises the amino acid sequence of SEQ ID NO:3.

In some cases, a light-responsive protein comprising a core amino acid sequence at least about 90%, 91%, 92%, 93%, 94%, 95%, 96%, 97%, 98%, 99%, or 100% identical to the sequence shown in SEQ ID NO:1, an ER export signal, and a membrane trafficking signal, can be used to generate a non-human animal model of the present disclosure.

Further disclosure related to light-responsive chloride pump proteins can be found in U.S. Patent Application Publication Nos: 2009/0093403 and 2010/0145418, and International Patent Publication No. WO 2011/116238, the disclosures of each of which are hereby incorporated by reference in their entireties.

### Light-Responsive Proton Pumps

In some aspects, one or more light-responsive proton pumps are expressed on the plasma membranes of a neuron in a non-human animal model of the present disclosure. In some embodiments, the light-responsive proton pump protein can be responsive to blue light and can be derived from Guillardia theta, wherein the proton pump protein can be capable of mediating a hyperpolarizing current in the cell when the cell is illuminated with blue light. The light can have a wavelength between about 450 and about 495 nm or can have a wavelength of about 490 nm. In another embodiment, the light-responsive proton pump protein can comprise an amino acid sequence at least about 90%, 91%, 92%, 93%, 94%, 95%, 96%, 97%, 98%, 99%, or 100% identical to the sequence shown in SEQ ID NO:4. The light-responsive proton pump protein can additionally comprise substitutions, deletions, and/or insertions introduced into a native amino acid sequence to increase or decrease sensitivity to light, increase or decrease sensitivity to particular wavelengths of light, and/or increase or decrease the ability of the light-responsive proton pump protein to regulate the polarization state of the plasma membrane of the cell. Additionally, the light-responsive proton pump protein can contain one or more conservative amino acid substitutions and/or one or more non-conservative amino acid substitutions. The light-responsive proton pump protein comprising substitutions, deletions, and/or insertions introduced into the native amino acid sequence suitably retains the ability to hyperpolarize the plasma membrane of a neuronal cell in response to light.

In other aspects of the methods disclosed herein, the light-responsive proton pump protein can comprise a core amino acid sequence at least about 90%, 91%, 92%, 93%, 94%, 95%, 96%, 97%, 98%, 99%, or 100% identical to the sequence shown in SEQ ID NO:4 and at least one (such as one, two, three, or more) amino acid sequence motifs which enhance transport to the plasma membranes of mammalian cells selected from the group consisting of a signal peptide, an ER export signal, and a membrane trafficking signal. In some embodiments, the light-responsive proton pump protein comprises an N-terminal signal peptide and a C-terminal ER export signal. In some embodiments, the light-responsive proton pump protein comprises an N-terminal signal peptide and a C-terminal trafficking signal. In some embodiments, the light-responsive proton pump protein comprises an N-terminal signal peptide, a C-terminal ER Export signal, and a C-terminal trafficking signal. In some embodiments, the light-responsive proton pump protein comprises a C-terminal ER Export signal and a C-terminal trafficking signal. In some embodiments, the C-terminal ER Export signal and the C-terminal trafficking signal are linked by a linker. The linker can comprise any of about 5, 10, 20, 30, 40, 50, 75, 100, 125, 150, 175, 200, 225, 250, 275, 300, 400, or 500 amino acids in length. The linker may further comprise a fluorescent protein, for example, but not limited to, a yellow fluorescent protein, a red fluorescent protein, a green fluorescent protein, or a cyan fluorescent protein. In some embodiments the ER Export signal is more C-terminally located than the trafficking signal. In some embodiments the trafficking signal is more C-terminally located than the ER Export signal.

Further disclosure related to light-responsive proton pump proteins can be found in International Patent Application No. PCT/US2011/028893, the disclosure of which is hereby incorporated by reference in its entirety.

### Light-Responsive Cation Channel Proteins

In some aspects, one or more light-responsive cation channels is expressed on the plasma membranes of a neuron in a subject non-human animal model. In some aspects, the light-responsive cation channel protein can be derived from Chlamydomonas reinhardtii, wherein the cation channel protein can be capable of mediating a depolarizing current in the cell when the cell is illuminated with light. In another embodiment, the light-responsive cation channel protein can comprise an amino acid sequence at least about 90%, 91%, 92%, 93%, 94%, 95%, 96%, 97%, 98%, 99%, or 100% identical to the sequence shown in SEQ ID NO:5. The light used to activate the light-responsive cation channel protein derived from Chlamydomonas reinhardtii can have a wavelength between about 460 and about 495 nm or can have a wavelength of about 480 nm. Additionally, the light can have an intensity of at least about 100 Hz. In some embodiments, activation of the light-responsive cation channel derived from Chlamydomonas reinhardtii with light having an intensity of 100 Hz can cause depolarization-induced synaptic depletion of the neurons expressing the light-responsive cation channel. The light-responsive cation channel protein can additionally comprise substitutions, deletions, and/or insertions introduced into a native amino acid sequence to increase or decrease sensitivity to light, increase or decrease sensitivity to particular wavelengths of light, and/or increase or decrease the ability of the light-responsive cation channel protein to regulate the polarization state of the plasma membrane of the cell. Additionally, the light-responsive cation channel protein can contain one or more conservative amino acid substitutions and/or one or more non-conservative amino acid substitutions. The light-responsive proton pump protein comprising substitutions, deletions, and/or insertions introduced into the native amino acid sequence suitably retains the ability to depolarize the plasma membrane of a neuronal cell in response to light.

Further disclosure related to light-responsive cation channel proteins can be found in U.S. Patent Application Publication No. 2007/0054319 and International Patent Application Publication Nos. WO 2009/131837 and WO 2007/024391, the disclosures of each of which are hereby incorporated by reference in their entireties.

### Step Function Opsins and Stabilized Step Function Opsins

In some cases, the light-responsive cation channel protein can be a step function opsin (SFO) protein or a stabilized step function opsin (SSFO) protein that can have specific amino acid substitutions at key positions throughout the retinal binding pocket of the protein. In some embodiments, the SFO protein can have a mutation at amino acid residue C128 of SEQ ID NO:5. In other embodiments, the SFO protein has a C128A mutation in SEQ ID NO:5. In other embodiments, the SFO protein has a C128S mutation in SEQ ID NO:5. In another embodiment, the SFO protein has a C128T mutation in SEQ ID NO:5. In some embodiments, the SFO protein can comprise an amino acid sequence at least about 90%, 91%, 92%, 93%, 94%, 95%, 96%, 97%, 98%, 99%, or 100% identical to the sequence shown in SEQ ID NO:6.

In some embodiments, the SSFO protein can have a mutation at amino acid residue D156 of SEQ ID NO:5. In other embodiments, the SSFO protein can have a mutation at both amino acid residues C128 and D156 of SEQ ID NO:5. In one embodiment, the SSFO protein has an C128S and a D156A mutation in SEQ ID NO:5. In another embodiment, the SSFO protein can comprise an amino acid sequence at least about 90%, 91%, 92%, 93%, 94%, 95%, 96%, 97%, 98%, 99%, or 100% identical to the sequence shown in SEQ ID NO:7.

In some embodiments the SFO or SSFO protein is capable of mediating a depolarizing current in the cell when the cell is illuminated with blue light. In other embodiments, the light can have a wavelength of about 445 nm. Additionally, the light can have an intensity of about 100 Hz. In some embodiments, activation of the SFO or SSFO protein with light having an intensity of 100 Hz can cause depolarization-induced synaptic depletion of the neurons expressing the SFO or SSFO protein. In some embodiments, each of the disclosed step function opsin and stabilized step function opsin proteins can have specific properties and characteristics for use in depolarizing the membrane of a neuronal cell in response to light.

Further disclosure related to SFO or SSFO proteins can be found in International Patent Application Publication No. WO 2010/056970 and U.S. Provisional Patent Application Nos. 61/410,704 and 61/511,905, the disclosures of each of which are hereby incorporated by reference in their entireties.

### C1V1 Chimeric Cation Channels

In some cases, the light-responsive cation channel protein can be a C1V1 chimeric protein derived from the VChR1 protein of Volvox carteri and the ChR1 protein from Chlamydomonas reinhardti, wherein the protein comprises the amino acid sequence of VChR1 having at least the first and second transmembrane helices replaced by the first and second transmembrane helices of ChR1; is responsive to light; and is capable of mediating a depolarizing current in the cell when the cell is illuminated with light. In some embodiments, the C1V1 protein can further comprise a replacement within the intracellular loop domain located between the second and third transmembrane helices of the chimeric light responsive protein, wherein at least a portion of the intracellular loop domain is replaced by the corresponding portion from ChR1. In another embodiment, the portion of the intracellular loop domain of the C1V1 chimeric protein can be replaced with the corresponding portion from ChR1 extending to amino acid residue A145 of the ChR1. In other embodiments, the C1V1 chimeric protein can further comprise a replacement within the third transmembrane helix of the chimeric light responsive protein, wherein at least a portion of the third transmembrane helix is replaced by the corresponding sequence of ChR1. In yet another embodiment, the portion of the intracellular loop domain of the C1V1 chimeric protein can be replaced with the corresponding portion from ChR1 extending to amino acid residue W163 of the ChR1. In other embodiments, the C1V1 chimeric protein can comprise an amino acid sequence at least about 90%, 91%, 92%, 93%, 94%, 95%, 96%, 97%, 98%, 99%, or 100% identical to the sequence shown in SEQ ID NO:8.

In some embodiments, the C1V1 protein can mediate a depolarizing current in the cell when the cell is illuminated with green light. In other embodiments, the light can have a wavelength of between about 540 nm to about 560 nm. In some embodiments, the light can have a wavelength of about 542 nm. In some embodiments, the C1V1 chimeric protein is not capable of mediating a depolarizing current in the cell when the cell is illuminated with violet light. In some embodiments, the chimeric protein is not capable of mediating a depolarizing current in the cell when the cell is illuminated with light having a wavelength of about 405 nm. Additionally, the light can have an intensity of about 100 Hz. In some embodiments, activation of the C1V1 chimeric protein with light having an intensity of 100 Hz can cause depolarization-induced synaptic depletion of the neurons expressing the C1V1 chimeric protein. In some embodiments, the disclosed C1V1 chimeric protein can have specific properties and characteristics for use in depolarizing the membrane of a neuronal cell in response to light.

### C1V1 Chimeric Mutant Variants

In some light-responsive opsins suitable for use can comprise substituted or mutated amino acid sequences, wherein the mutant polypeptide retains the characteristic light-responsive nature of the precursor C1V1 chimeric polypeptide but may also possess altered properties in some specific aspects. For example, the mutant light-responsive C1V1 chimeric proteins can exhibit an increased level of expression both within an animal cell or on the animal cell plasma membrane; an altered responsiveness when exposed to different wavelengths of light, particularly red light; and/or a combination of traits whereby the chimeric C1V1 polypeptide possess the properties of low desensitization, fast deactivation, low violet-light activation for minimal cross-activation with other light-responsive cation channels, and/or strong expression in animal cells.

For example, C1V1 chimeric light-responsive opsin proteins that can have specific amino acid substitutions at key positions throughout the retinal binding pocket of the VChR1 portion of the chimeric polypeptide are suitable for use. In some embodiments, the C1V1 protein can have a mutation at amino acid residue E122 of SEQ ID NO:7. In some embodiments, the C1V1 protein can have a mutation at amino acid residue E162 of SEQ ID NO:7. In other embodiments, the C1V1 protein can have a mutation at both amino acid residues E162 and E122 of SEQ ID NO:7. In other embodiments, the C1V1 protein can comprise an amino acid sequence at least about 90%, 91%, 92%, 93%, 94%, 95%, 96%, 97%, 98%, 99%, or 100% identical to the sequence shown in SEQ ID NO:9, SEQ ID NO:10, or SEQ ID NO:11. In some embodiments, each of the disclosed mutant C1V1 chimeric proteins can have specific properties and characteristics for use in depolarizing the membrane of an animal cell in response to light.

In some aspects, the C1V1-E122 mutant chimeric protein is capable of mediating a depolarizing current in the cell when the cell is illuminated with light. In some embodiments the light can be green light. In other embodiments, the light can have a wavelength of between about 540 nm to about 560 nm. In some embodiments, the light can have a wavelength of about 546 nm. In other embodiments, the C1V1-E122 mutant chimeric protein can mediate a depolarizing current in the cell when the cell is illuminated with red light. In some embodiments, the red light can have a wavelength of about 630 nm. In some embodiments, the C1V1-E122 mutant chimeric protein does not mediate a depolarizing current in the cell when the cell is illuminated with violet light. In some embodiments, the chimeric protein does not mediate a depolarizing current in the cell when the cell is illuminated with light having a wavelength of about 405 nm. Additionally, the light can have an intensity of about 100 Hz. In some embodiments, activation of the C1V1-E122 mutant chimeric protein with light having an intensity of 100 Hz can cause depolarization-induced synaptic depletion of the neurons expressing the C1V1-E122 mutant chimeric protein. In some embodiments, the disclosed C1V1-E122 mutant chimeric protein can have specific properties and characteristics for use in depolarizing the membrane of a neuronal cell in response to light.

In other aspects, the C1V1-E162 mutant chimeric protein is capable of mediating a depolarizing current in the cell when the cell is illuminated with light. In some embodiments the light can be green light. In other embodiments, the light can have a wavelength of between about 540 nm to about 535 nm. In some embodiments, the light can have a wavelength of about 542 nm. In other embodiments, the light can have a wavelength of about 530 nm. In some embodiments, the C1V1-E162 mutant chimeric protein does not mediate a depolarizing current in the cell when the cell is illuminated with violet light. In some embodiments, the chimeric protein does not mediate a depolarizing current in the cell when the cell is illuminated with light having a wavelength of about 405 nm. Additionally, the light can have an intensity of about 100 Hz. In some embodiments, activation of the C1V1-E162 mutant chimeric protein with light having an intensity of 100 Hz can cause depolarization-induced synaptic depletion of the neurons expressing the C1V1-E162 mutant chimeric protein. In some embodiments, the disclosed C1V1-E162 mutant chimeric protein can have specific properties and characteristics for use in depolarizing the membrane of a neuronal cell in response to light.

In yet other aspects, the C1V1-E122/E162 mutant chimeric protein is capable of mediating a depolarizing current in the cell when the cell is illuminated with light. In some embodiments the light can be green light. In other embodiments, the light can have a wavelength of between about 540 nm to about 560 nm. In some embodiments, the light can have a wavelength of about 546 nm. In some embodiments, the C1V1-E122/E162 mutant chimeric protein does not mediate a depolarizing current in the cell when the cell is illuminated with violet light. In some embodiments, the chimeric protein does not mediate a depolarizing current in the cell when the cell is illuminated with light having a wavelength of about 405 nm. In some embodiments, the C1V1-E122/E162 mutant chimeric protein can exhibit less activation when exposed to violet light relative to C1V1 chimeric proteins lacking mutations at E122/E162 or relative to other light-responsive cation channel proteins. Additionally, the light can have an intensity of about 100 Hz. In some embodiments, activation of the C1V1-E122/E162 mutant chimeric protein with light having an intensity of 100 Hz can cause depolarization-induced synaptic depletion of the neurons expressing the C1V1-E122/E162 mutant chimeric protein. In some embodiments, the disclosed C1V1-E122/E162 mutant chimeric protein can have specific properties and characteristics for use in depolarizing the membrane of a neuronal cell in response to light.

Further disclosure related to C1V1 chimeric cation channels as well as mutant variants of the same can be found in U.S. Provisional Patent Application Nos. 61/410,736, 61/410,744, and 61/511,912, the disclosures of each of which are hereby incorporated by reference in their entireties.

### Sequences

The amino acid sequence of NpHR without the signal peptide:

(SEQ ID NO: 1)

VTQRELFEFVLNDPLLASSLYINIALAGLSILLFVFMTRGLDDPRAKLIA
VSTILVPVVSIASYTGLASGLTISVLEMPAGHFAEGSSVMLGGEEVDGVV
TMWGRYLTWALSTPMILLALGLLAGSNATKLFTAITFDIAMCVTGLAAAL
TTSSHLMRWFWYAISCACFLVVLYILLVEWAQDAKAAGTADMFNTLKLLT
VVMWLGYPIVWALGVEGIAVLPVGVTSWGYSFLDIVAKYIFAFLLLNYLT
SNESVVSGSILDVPSASGTPADD

The amino acid sequence of eYFP-NpHR3.0:

(SEQ ID NO: 2)

MTETLPPVTESAVALQAEVTQRELFEFVLNDPLLASSLYINIALAGLSIL
LFVFMTRGLDDPRAKLIAVSTILVPVVSIASYTGLASGLTISVLEMPAGH
FAEGSSVMLGGEEVDGVVTMWGRYLTWALSTPMILLALGLLAGSNATKLF
TAITFDIAMCVTGLAAALTTSSHLMRWFWYAISCACFLVVLYILLVEWAQ
DAKAAGTADMFNTLKLLTVVMWLGYPIVWALGVEGIAVLPVGVTSWGYSF
LDIVAKYIFAFLLLNYLTSNESVVSGSILDVPSASGTPADDAAAKSRITS
EGEYIPLDQIDINVVSKGEELFTGVVPILVELDGDVNGHKFSVSGEGEGD
ATYGKLTLKFICTTGKLPVPWPTLVTTFGYGLQCFARYPDHMKQHDFFKS
AMPEGYVQERTIFFKDDGNYKTRAEVKFEGDTLVNRIELKGIDFKEDGNI
LGHKLEYNYNSHNVYIMADKQKNGIKVNFKIRHNIEDGSVQLADHYQQNT
PIGDGPVLLPDNHYLSYQSALSKDPNEKRDHMVLLEFVTAAGITLGMDEL
YKFCYENEV

The amino acid sequence of eYFP-NpHR3.1:

(SEQ ID NO: 3)

MVTQRELFEFVLNDPLLASSLYINIALAGLSILLFVFMTRGLDDPRAKLI
AVSTILVPVVSIASYTGLASGLTISVLEMPAGHFAEGSSVMLGGEEVDGV
VTMWGRYLTWALSTPMILLALGLLAGSNATKLFTAITFDIAMCVTGLAAA
LTTSSHLMRWFWYAISCACFLVVLYILLVEWAQDAKAAGTADMFNTLKLL
TVVMWLGYPIVWALGVEGIAVLPVGVTSWGYSFLDIVAKYIFAFLLLNYL
TSNESVVSGSILDVPSASGTPADDAAAKSRITSEGEYIPLDQIDINVVSK
GEELFTGVVPILVELDGDVNGHKFSVSGEGEGDATYGKLTLKFICTTGKL
PVPWPTLVTTFGYGLQCFARYPDHMKQHDFFKSAMPEGYVQERTIFFKDD
GNYKTRAEVKFEGDTLVNRIELKGIDFKEDGNILGHKLEYNYNSHNVYIM
ADKQKNGIKVNFKIRHNIEDGSVQLADHYQQNTPIGDGPVLLPDNHYLSY
QSALSKDPNEKRDHMVLLEFVTAAGITLGMDELYKFCYENEV

The amino acid sequence of GtR3:

(SEQ ID NO: 4)

ASSFGKALLEFVFIVFACITLLLGINAAKSKAASRVLFPATFVTGIASIA
YFSMASGGGWVIAPDCRQLFVARYLDWLITTPLLLIDLGLVAGVSRWDIM
ALCLSDVLMIATGAFGSLTVGNVKWVWWFFGMCWFLHIIFALGKSWAEAA
KAKGGDSASVYSKIAGITVITWFCYPVVWVFAEGFGNFSVTFEVLIYGVL
DVISKAVFGLILMSGAATGYESI

The amino acid sequence of ChR2:

(SEQ ID NO: 5)

MDYGGALSAVGRELLFVTNPVVVNGSVLVPEDQCYCAGWIESRGTNGAQT
ASNVLQWLAAGFSILLLMFYAYQTWKSTCGWEEIYVCAIEMVKVILEFFF
EFKNPSMLYLATGHRVQWLRYAEWLLTCPVILIHLSNLTGLSNDYSRRTM
GLLVSDIGTIVWGATSAMATGYVKVIFFCLGLCYGANTFFHAAKAYIEGY
HTVPKGRCRQVVTGMAWLFFVSWGMFPILFILGPEGFGVLSVYGSTVGHT
IIDLMSKNCWGLLGHYLRVLIHEHILIHGDIRKTTKLNIGGTEIEVETLV
EDEAEAGAVP

The amino acid sequence of SFO:

(SEQ ID NO: 6)

MDYGGALSAVGRELLFVTNPVVVNGSVLVPEDQCYCAGWIESRGTNGAQT
ASNVLQWLAAGFSILLLMFYAYQTWKSTCGWEEIYVCAIEMVKVILEFFF
EFKNPSMLYLATGHRVQWLRYAEWLLTSPVILIHLSNLTGLSNDYSRRTM
GLLVSDIGTIVWGATSAMATGYVKVIFFCLGLCYGANTFFHAAKAYIEGY
HTVPKGRCRQVVTGMAWLFFVSWGMFPILFILGPEGFGVLSVYGSTVGHT
IIDLMSKNCWGLLGHYLRVLIHEHILIHGDIRKTTKLNIGGTEIEVETLV
EDEAEAGAVP

The amino acid sequence of SSFO:

(SEQ ID NO: 7)

MDYGGALSAVGRELLFVTNPVVVNGSVLVPEDQCYCAGWIESRGTNGAQT
ASNVLQWLAAGFSILLLMFYAYQTWKSTCGWEEIYVCAIEMVKVILEFFF
EFKNPSMLYLATGHRVQWLRYAEWLLTSPVILIHLSNLTGLSNDYSRRTM
GLLVSAIGTIVWGATSAMATGYVKVIFFCLGLCYGANTFFHAAKAYIEGY
HTVPKGRCRQVVTGMAWLFFVSWGMFPILFILGPEGFGVLSVYGSTVGHT
IIDLMSKNCWGLLGHYLRVLIHEHILIHGDIRKTTKLNIGGTEIEVETLV
EDEAEAGAVP

The amino acid sequence of C1V1:

(SEQ ID NO: 8)

MSRRPWLLALALAVALAAGSAGASTGSDATVPVATQDGPDYVFHRAHERM
LFQTSYTLENNGSVICIPNNGQCFCLAWLKSNGTNAEKLAANILQWITFA
LSALCLMFYGYQTWKSTCGWEEIYVATIEMIKFIIEYFHEFDEPAVIYSS
NGNKTVWLRYAEWLLTCPVLLIHLSNLTGLKDDYSKRTMGLLVSDVGCIV
WGATSAMCTGWTKILFFLISLSYGMYTYFHAAKVYIEAFHTVPKGICREL
VRVMAWTFFVAWGMFPVLFLLGTEGFGHISPYGSAIGHSILDLIAKNMWG
VLGNYLRVKIHEHILLYGDIRKKQKITIAGQEMEVETLVAEEED

The amino acid sequence of C1V1 (E122T):

(SEQ ID NO: 9)

MSRRPWLLALALAVALAAGSAGASTGSDATVPVATQDGPDYVFHRAHERM
LFQTSYTLENNGSVICIPNNGQCFCLAWLKSNGTNAEKLAANILQWITFA
LSALCLMFYGYQTWKSTCGWETIYVATIEMIKFIIEYFHEFDEPAVIYSS
NGNKTVWLRYAEWLLTCPVLLIHLSNLTGLKDDYSKRTMGLLVSDVGCIV
WGATSAMCTGWTKILFFLISLSYGMYTYFHAAKVYIEAFHTVPKGICREL
VRVMAWTFFVAWGMFPVLFLLGTEGFGHISPYGSAIGHSILDLIAKNMWG
VLGNYLRVKIHEHILLYGDIRKKQKITIAGQEMEVETLVAEEED

The amino acid sequence of C1V1 (E162T):

(SEQ ID NO: 10)

MSRRPWLLALALAVALAAGSAGASTGSDATVPVATQDGPDYVFHRAHERM
LFQTSYTLENNGSVICIPNNGQCFCLAWLKSNGTNAEKLAANILQWITFA
LSALCLMFYGYQTWKSTCGWEEIYVATIEMIKFIIEYFHEFDEPAVIYSS
NGNKTVWLRYATWLLTCPVLLIHLSNLTGLKDDYSKRTMGLLVSDVGCIV
WGATSAMCTGWTKILFFLISLSYGMYTYFHAAKVYIEAFHTVPKGICREL
VRVMAWTFFVAWGMFPVLFLLGTEGFGHISPYGSAIGHSILDLIAKNMWG
VLGNYLRVKIHEHILLYGDIRKKQKITIAGQEMEVETLVAEEED

The amino acid sequence of C1V1 (E122T/E162T):

(SEQ ID NO: 11)

MSRRPWLLALALAVALAAGSAGASTGSDATVPVATQDGPDYVFHRAHERM
LFQTSYTLENNGSVICIPNNGQCFCLAWLKSNGTNAEKLAANILQWITFA
LSALCLMFYGYQTWKSTCGWETIYVATIEMIKFIIEYFHEFDEPAVIYSS
NGNKTVWLRYATWLLTCPVLLIHLSNLTGLKDDYSKRTMGLLVSDVGCIV
WGATSAMCTGWTKILFFLISLSYGMYTYFHAAKVYIEAFHTVPKGICREL
VRVMAWTFFVAWGMFPVLFLLGTEGFGHISPYGSAIGHSILDLIAKNMWG
VLGNYLRVKIHEHILLYGDIRKKQKITIAGQEMEVETLVAEEED

### Modifications

A light-responsive opsin can comprise various modifications, e.g., the addition of one or more amino acid sequence motifs that enhance transport to the plasma membranes of mammalian cells. Light-responsive opsin proteins having components derived from evolutionarily simpler organisms may not be expressed or tolerated by mammalian cells or may exhibit impaired subcellular localization when expressed at high levels in mammalian cells. Consequently, in some embodiments, the light-responsive opsin proteins expressed in a cell can be fused to one or more amino acid sequence motifs selected from the group consisting of a signal peptide, an endoplasmic reticulum (ER) export signal, a membrane trafficking signal, and/or an N-terminal Golgi export signal. The one or more amino acid sequence motifs which enhance light-responsive protein transport to the plasma membranes of mammalian cells can be fused to the N-terminus, the C-terminus, or to both the N- and C-terminal ends of the light-responsive protein. Optionally, the light-responsive protein and the one or more amino acid sequence motifs may be separated by a linker. In some embodiments, the light-responsive protein can be modified by the addition of a trafficking signal (ts) which enhances transport of the protein to the cell plasma membrane. In some embodiments, the trafficking signal can be derived from the amino acid sequence of the human inward rectifier potassium channel Kir2.1. In other embodiments, the trafficking signal can comprise the amino acid sequence KSRITSEGEYIPLDQIDINV (SEQ ID NO:17).

Trafficking sequences that are suitable for use can comprise an amino acid sequence having 90%, 91%, 92%, 93%, 94%, 95%, 96%, 97%, 98%, 99%, or 100%, amino acid sequence identity to an amino acid sequence such a trafficking sequence of human inward rectifier potassium channel Kir2.1 (e.g., KSRITSEGEYIPLDQIDINV; SEQ ID NO:17).

A trafficking sequence can have a length of from about 10 amino acids to about 50 amino acids, e.g., from about 10 amino acids to about 20 amino acids, from about 20 amino acids to about 30 amino acids, from about 30 amino acids to about 40 amino acids, or from about 40 amino acids to about 50 amino acids.

Signal sequences that are suitable for use can comprise an amino acid sequence having 90%, 91%, 92%, 93%, 94%, 95%, 96%, 97%, 98%, 99%, or 100%, amino acid sequence identity to an amino acid sequence such as one of the following:

1) the signal peptide of hChR2 (e.g., MDYGGALSAVGRELLFVTNPVVVNGS; SEQ ID NO:19);

2) the 132 subunit signal peptide of the neuronal nicotinic acetylcholine receptor (e.g., MAGHSNSMALFSFSLLWLCSGVLGTEF; SEQ ID NO:20);

3) a nicotinic acetylcholine receptor signal sequence (e.g., MGLRALMLWLLAAAGLVRESLQG; SEQ ID NO:21); and

4) a nicotinic acetylcholine receptor signal sequence (e.g., MRGTPLLLVVSLFSLLQD; SEQ ID NO:22).

A signal sequence can have a length of from about 10 amino acids to about 50 amino acids, e.g., from about 10 amino acids to about 20 amino acids, from about 20 amino acids to about 30 amino acids, from about 30 amino acids to about 40 amino acids, or from about 40 amino acids to about 50 amino acids.

Endoplasmic reticulum (ER) export sequences that are suitable for use in a modified opsin of the present disclosure include, e.g., VXXSL (where X is any amino acid) (e.g., VKESL (SEQ ID NO:14); VLGSL (SEQ ID NO:15); etc.); NANSFCYENEVALTSK (SEQ ID NO:16); FXYENE (SEQ ID NO:12) where X is any amino acid), e.g., FCYENEV (SEQ ID NO:13); and the like. An ER export sequence can have a length of from about 5 amino acids to about 25 amino acids, e.g., from about 5 amino acids to about 10 amino acids, from about 10 amino acids to about 15 amino acids, from about 15 amino acids to about 20 amino acids, or from about 20 amino acids to about 25 amino acids.

Additional protein motifs which can enhance light-responsive protein transport to the plasma membrane of a cell are described in U.S. patent application Ser. No. 12/041,628, which is incorporated herein by reference in its entirety. In some embodiments, the signal peptide sequence in the protein can be deleted or substituted with a signal peptide sequence from a different protein.

### Fusions

In some cases, a light-activated opsin is a fusion protein, e.g., a light-activated opsin comprises heterologous amino acids (e.g., a fusion partner), e.g., at the amino terminus and/or at the carboxyl terminus and/or internally to the light-activated opsin. For example, a fusion protein can include a light-activated opsin and a fusion partner, where suitable fusion partners include, enzymes, fluorescent proteins, epitope tags, and the like.

Suitable fluorescent proteins that can be linked to a subject antibody include, but are not limited to, a green fluorescent protein (GFP) from Aequoria victoria or a mutant or derivative thereof e.g., as described in U.S. Pat. Nos. 6,066,476; 6,020,192; 5,985,577; 5,976,796; 5,968,750; 5,968,738; 5,958,713; 5,919,445; 5,874,304; e.g., Enhanced GFP, many such GFP which are available commercially, e.g., from Clontech, Inc.; a red fluorescent protein; a yellow fluorescent protein (YFP); any of a variety of fluorescent and colored proteins from Anthozoan species, as described in, e.g., Matz et al. (1999) Nature Biotechnol. 17:969-973; mCherry; enhanced GFP, enhanced YFP; and the like.

### Nucleic Acids

A polynucleotide comprising a nucleotide sequence encoding a light-responsive protein can be used to generate a subject non-human animal model. In some embodiments, the polynucleotide comprises an expression cassette. In some embodiments, the polynucleotide is a vector comprising the above-described nucleic acid. In some embodiments, the nucleic acid encoding a light-responsive opsin is operably linked to a promoter. Promoters are well known in the art. Any promoter that functions in the host cell can be used for expression of the light-responsive opsin proteins and/or any variant thereof. In one embodiment, the promoter used to drive expression of the light-responsive opsin proteins can be a promoter that is specific to dopaminergic neurons. In other embodiments, the promoter is capable of driving expression of the light-responsive opsin proteins in excitatory neurons. Initiation control regions or promoters, which are useful to drive expression of the light-responsive opsin proteins or variant thereof in a specific animal cell are numerous and familiar to those skilled in the art. Virtually any promoter capable of driving these nucleic acids can be used.

Neuron-specific promoters and other control elements (e.g., enhancers) are known in the art. Suitable neuron-specific control sequences include, but are not limited to, a neuron-specific enolase (NSE) promoter (see, e.g., EMBL HSENO2, X51956); an aromatic amino acid decarboxylase (AADC) promoter; a neurofilament promoter (see, e.g., GenBank HUMNFL, L04147); a synapsin promoter (see, e.g., GenBank HUMSYNIB, M55301); a thy-1 promoter (see, e.g., Chen et al. (1987) Cell 51:7-19; and Llewellyn, et al. (2010) Nat. Med. 16(10):1161-1166); a serotonin receptor promoter (see, e.g., GenBank S62283); a tyrosine hydroxylase promoter (TH) (see, e.g., Oh et al. (2009) Gene Ther 16:437; Sasaoka et al. (1992) Mol. Brain Res. 16:274; Boundy et al. (1998) J. Neurosci. 18:9989; and Kaneda et al. (1991) Neuron 6:583-594); a GnRH promoter (see, e.g., Radovick et al. (1991) Proc. Natl. Acad. Sci. USA 88:3402-3406); an L7 promoter (see, e.g., Oberdick et al. (1990) Science 248:223-226); a DNMT promoter (see, e.g., Bartge et al. (1988) Proc. Natl. Acad. Sci. USA 85:3648-3652); an enkephalin promoter (see, e.g., Comb et al. (1988) EMBO J. 17:3793-3805); a myelin basic protein (MBP) promoter; a Ca²⁺-calmodulin-dependent protein kinase II-alpha (CamKIIα) promoter (see, e.g., Mayford et al. (1996) Proc. Natl. Acad. Sci. USA 93:13250; and Casanova et al. (2001) Genesis 31:37); and a CMV enhancer/platelet-derived growth factor-β promoter (see, e.g., Liu et al. (2004) Gene Therapy 11:52-60).

In some embodiments, the promoter used to drive expression of the light-responsive protein can be a Thyl promoter, which is capable of driving robust expression of transgenes in neurons (See, e.g., Llewellyn, et al. (2010) Nat. Med. 16(10):1161-1166). In other embodiments, the promoter used to drive expression of the light-responsive protein can be an EF1α promoter, a cytomegalovirus (CMV) promoter, the CAG promoter, the synapsin promoter, or any other ubiquitous promoter capable of driving expression of the light-responsive opsin proteins in a neuron of a mammal.

In some cases, the nucleic acid is an expression vector comprising a transgene (e.g., a nucleotide sequence encoding a light-responsive protein or any variant thereof described herein). The vectors that can be administered include vectors comprising a nucleotide sequence which encodes an RNA (e.g., an mRNA) that when transcribed from the polynucleotides of the vector will result in the accumulation of light-responsive opsin proteins on the plasma membranes of target animal cells. Vectors which may be used, include, without limitation, lentiviral, herpes simplex virus (HSV), adenoviral, and adeno-associated viral (AAV) vectors. Lentiviruses include, but are not limited to HIV-1, HIV-2, SIV, FIV and EIAV. Lentiviruses may be pseudotyped with the envelope proteins of other viruses, including, but not limited to VSV, rabies, Mo-MLV, baculovirus and Ebola. Such vectors may be prepared using standard methods in the art.

In some embodiments, the vector is a recombinant AAV vector. AAV vectors are DNA viruses of relatively small size that can integrate, in a stable and site-specific manner, into the genome of the cells that they infect. They are able to infect a wide spectrum of cells without inducing any effects on cellular growth, morphology or differentiation, and they do not appear to be involved in human pathologies. The AAV genome has been cloned, sequenced and characterized. It encompasses approximately 4700 bases and contains an inverted terminal repeat (ITR) region of approximately 145 bases at each end, which serves as an origin of replication for the virus. The remainder of the genome is divided into two essential regions that carry the encapsidation functions: the left-hand part of the genome, that contains the rep gene involved in viral replication and expression of the viral genes; and the right-hand part of the genome, that contains the cap gene encoding the capsid proteins of the virus.

AAV vectors may be prepared using standard methods in the art. Adeno-associated viruses of any serotype are suitable (see, e.g., Blacklow, pp. 165-174 of “Parvoviruses and Human Disease” J. R. Pattison, ed. (1988); Rose, Comprehensive Virology 3:1, 1974; P. Tattersall “The Evolution of Parvovirus Taxonomy” In Parvoviruses (J R Kerr, S F Cotmore, M E Bloom, R M Linden, C R Parrish, Eds.) p 5-14, Hudder Arnold, London, UK (2006); and D E Bowles, J E Rabinowitz, R J Samulski “The Genus Dependovirus” (J R Kerr, S F Cotmore, M E Bloom, R M Linden, C R Parrish, Eds.) p 15-23, Hudder Arnold, London, UK (2006), the disclosures of each of which are hereby incorporated by reference herein in their entireties). Methods for purifying for vectors may be found in, for example, U.S. Pat. Nos. 6,566,118, 6,989,264, and 6,995,006 and WO/1999/011764 titled “Methods for Generating High Titer Helper-free Preparation of Recombinant AAV Vectors”, the disclosures of which are herein incorporated by reference in their entirety. Preparation of hybrid vectors is described in, for example, PCT Application No. PCT/US2005/027091, the disclosure of which is herein incorporated by reference in its entirety.

The use of vectors derived from the AAVs for transferring genes in vitro and in vivo has been described (See e.g., International Patent Application Publication Nos.: 91/18088 and WO 93/09239; U.S. Pat. Nos. 4,797,368, 6,596,535, and 5,139,941; and European Patent No.: 0488528, all of which are hereby incorporated by reference herein in their entireties). These publications describe various AAV-derived constructs in which the rep and/or cap genes are deleted and replaced by a gene of interest, and the use of these constructs for transferring the gene of interest in vitro (into cultured cells) or in vivo (directly into an organism). The replication defective recombinant AAVs according to the invention can be prepared by co-transfecting a plasmid containing the nucleic acid sequence of interest flanked by two AAV inverted terminal repeat (ITR) regions, and a plasmid carrying the AAV encapsidation genes (rep and cap genes), into a cell line that is infected with a human helper virus (for example an adenovirus). The AAV recombinants that are produced are then purified by standard techniques.

In some embodiments, the vector(s) for use in generating a subject non-human animal model are encapsidated into a virus particle (e.g. AAV virus particle including, but not limited to, AAV1, AAV2, AAV3, AAV4, AAV5, AAV6, AAV7, AAV8, AAV9, AAV10, AAV11, AAV12, AAV13, AAV14, AAV15, and AAV16). Methods of producing such particles are known in the art and are described in U.S. Pat. No. 6,596,535, the disclosure of which is hereby incorporated by reference in its entirety.

### Delivery of Light-Responsive Opsin Proteins

In some aspects, a polynucleotide encoding the light-responsive opsin proteins disclosed herein (for example, an AAV vector) is delivered directly to the neurons of interest (e.g., dopaminergic neurons of the ventral tegmental area (VTA); excitatory (glutamaergic) neurons of the medial prefrontal cortex (mPFC)) with a needle, catheter, or related device, using neurosurgical techniques known in the art, such as by stereotactic injection (See, e.g., Stein et al., J. Virol, 73:3424-3429, 1999; Davidson et al., PNAS, 97:3428-3432, 2000; Davidson et al., Nat. Genet. 3:219-223, 1993; and Alisky & Davidson, Hum. Gene Ther. 11:2315-2329, 2000, the contents of each of which are hereby incorporated by reference herein in their entireties) or fluoroscopy. In some embodiments, the polynucleotide encoding the light-responsive opsin proteins disclosed herein (for example, an AAV1 vector) can be delivered to dopaminergic neurons of the VTA. In other embodiments, the polynucleotide encoding the light-responsive opsin proteins disclosed herein (for example, an AAV vector) can be delivered to excitatory (glutamaergic) neurons of the mPFC.

In some aspects, polynucleotides encoding the light-responsive opsin proteins disclosed herein (for example, an AAV vector) can be delivered directly to the neurons of interest with a needle, catheter, or related device, using neurosurgical techniques known in the art, such as by stereotactic injection or fluoroscopy.

Other methods to deliver the light-responsive opsin proteins to the neurons of interest can also be used, such as, but not limited to, transfection with ionic lipids or polymers; electroporation; optical transfection; impalefection (e.g., method of gene delivery using a nanomaterial such as carbon nanofibers, carbon nanotubes, nanowires, and the like; see, e.g., Melechko et al. (2004) Nano Letters 4(7): p. 1213-1219); or via gene gun.

In some embodiments, a viral vector, such adenovirus, AAV2, and Rabies glycoprotein-pseudotyped lentivirus, is used, where the viral vector is taken up by muscle cells and retrogradely transported to a neuron (See, e.g., Azzouz et al., 2009, Antioxid Redox Signal., 11(7):1523-34; Kaspar et al., 2003, Science, 301(5634):839-842; Manabe et al., 2002. Apoptosis, 7(4):329-334).

### Light and Electrical Sources

In some aspects of the invention, the light-responsive opsin proteins disclosed herein can be activated by an implantable light source (such as a light cuff) or an implantable electrode placed around or near neurons expressing the light-responsive opsin proteins or nerves controlling such neurons. Electrode cuffs and electrodes surgically placed around or near nerves for use in electrical stimulation of those nerves are well known in the art (See, for example, U.S. Pat. Nos. 4,602,624, 7,142,925 and 6,600,956 as well as U.S. Patent Publication Nos. 2008/0172116 and 2010/0094372, the disclosures of each of which are hereby incorporated by reference in their entireties). The light sources (such as a light cuff) or electrodes can be comprised of any useful composition or mixture of compositions, such as platinum or stainless steel, as are known in the art, and may be of any useful configuration for stimulating the light-responsive opsin proteins expressed in a neuron, or nerves controlling such a neurons. For example, where the light-responsive opsin is expressed in an excitatory (glutamaergic) neuron of the mPFC, the light source can be used to direct light onto the excitatory (glutamaergic) neuron of the mPFC that express a light-responsive opsin; or the light source can be used to direct light onto the dorsal raphe nucleus (DRN), which is one of several targets of projection from the mPRC.

The electrodes or implantable light source (such as a light cuff) may be placed around or near a light-responsive opsin-expressing neuron (e.g., a dopaminergic neuron of the VTA; or an excitatory neuron of the mPFC); or the electrodes or implantable light source may be placed around or near the DRN. Suitable brain regions for placement of an electrode or implantable light source can be identified those skilled in the art prior to placing the electrode or implantable light source around or near the brain regions using known techniques in the art.

The implantable light source (such as a light cuff) can comprise an inner body, the inner body having at least one means for generating light which is configured to a power source. In some embodiments, the power source can be an internal battery for powering the light-generating means. In another embodiment, the implantable light source can comprise an external antenna for receiving wirelessly transmitted electromagnetic energy from an external source for powering the light-generating means. The wirelessly transmitted electromagnetic energy can be a radio wave, a microwave, or any other electromagnetic energy source that can be transmitted from an external source to power the light-generating means of the implantable light source (such as a light cuff). In one embodiment, the light-generating means is controlled by an integrated circuit produced using semiconductor or other processes known in the art.

In some aspects, the light means can be a light emitting diode (LED). In some embodiments, the LED can generate blue and/or green light. In other embodiments, the LED can generate amber and/or yellow light. In some embodiments, several micro LEDs are embedded into the inner body of the implantable light source (such as a light cuff). In other embodiments, the light-generating means is a solid state laser diode or any other means capable of generating light. The light generating means can generate light having an intensity sufficient to activate the light-responsive opsin proteins expressed on the plasma membrane of the nerves in proximity to the light source (such as a light cuff). In some embodiments, the light-generating means produces light having an intensity of any of about 0.05 mW/mm², 0.1 mW/mm², 0.2 mW/mm², 0.3 mW/mm², 0.4 mW/mm², 0.5 mW/mm², about 0.6 mW/mm², about 0.7 mW/mm², about 0.8 mW/mm², about 0.9 mW/mm², about 1.0 mW/mm², about 1.1 mW/mm², about 1.2 mW/mm², about 1.3 mW/mm², about 1.4 mW/mm², about 1.5 mW/mm², about 1.6 mW/mm², about 1.7 mW/mm², about 1.8 mW/mm², about 1.9 mW/mm², about 2.0 mW/mm², about 2.1 mW/mm², about 2.2 mW/mm², about 2.3 mW/mm², about 2.4 mW/mm², about 2.5 mW/mm², about 3 mW/mm², about 3.5 mW/mm², about 4 mW/mm², about 4.5 mW/mm², about 5 mW/mm², about 5.5 mW/mm², about 6 mW/mm², about 7 mW/mm², about 8 mW/mm², about 9 mW/mm², or about 10 mW/mm², inclusive, including values in between these numbers. In other embodiments, the light-generating means produces light having an intensity of at least about 100 Hz.

In some aspects, the light-generating means can be externally activated by an external controller. The external controller can comprise a power generator which can be mounted to a transmitting coil. In some embodiments of the external controller, a battery can be connected to the power generator, for providing power thereto. A switch can be connected to the power generator, allowing an individual to manually activate or deactivate the power generator. In some embodiments, upon activation of the switch, the power generator can provide power to the light-generating means on the light source through electromagnetic coupling between the transmitting coil on the external controller and the external antenna of the implantable light source (such as a light cuff). The transmitting coil can establish an electromagnetic coupling with the external antenna of the implantable light source when in proximity thereof, for supplying power to the light-generating means and for transmitting one or more control signals to the implantable light source. In some embodiments, the electromagnetic coupling between the transmitting coil of the external controller and the external antenna of the implantable light source (such as a light cuff) can be radio-frequency magnetic inductance coupling. When radio-frequency magnetic inductance coupling is used, the operational frequency of the radio wave can be between about 1 and 20 MHz, inclusive, including any values in between these numbers (for example, about 1 MHz, about 2 MHz, about 3 MHz, about 4 MHz, about 5 MHz, about 6 MHz, about 7 MHz, about 8 MHz, about 9 MHz, about 10 MHz, about 11 MHz, about 12 MHz, about 13 MHz, about 14 MHz, about 15 MHz, about 16 MHz, about 17 MHz, about 18 MHz, about 19 MHz, or about 20 MHz). However, other coupling techniques may be used, such as an optical receiver, infrared, or a biomedical telemetry system (See, e.g., Kiourti, “Biomedical Telemetry: Communication between Implanted Devices and the External World, Opticon1826, (8): Spring, 2010).

### Screening Methods

The present disclosure provides methods of identifying an agent that treats depression in a mammal. The present disclosure also provides methods of identifying targets for therapeutic intervention in the treatment of depression. The present disclosure also provides methods of identifying drugs that are under development for treatment of a disorder, which drugs could induce depression in an individual.

As used herein, the term “determining” refers to both quantitative and qualitative determinations and as such, the term “determining” is used interchangeably herein with “assaying,” “measuring,” and the like.

The terms “candidate agent,” “test agent,” “agent”, “substance” and “compound” are used interchangeably herein. Candidate agents encompass numerous chemical classes, typically synthetic, semi-synthetic, or naturally occurring inorganic or organic molecules. Candidate agents include those found in large libraries of synthetic or natural compounds. For example, synthetic compound libraries are commercially available from Maybridge Chemical Co. (Trevillet, Cornwall, UK), ComGenex (South San Francisco, Calif.), and MicroSource (New Milford, Conn.). A rare chemical library is available from Aldrich (Milwaukee, Wis.) and can also be used. Alternatively, libraries of natural compounds in the form of bacterial, fungal, plant and animal extracts are available from Pan Labs (Bothell, Wash.) or are readily producible.

Candidate agents can be small organic or inorganic compounds having a molecular weight of more than 50 daltons and less than about 2,500 daltons. Candidate agents can comprise functional groups necessary for structural interaction with proteins, e.g., hydrogen bonding, and may include at least an amine, carbonyl, hydroxyl or carboxyl group, and may contain at least two of the functional chemical groups. The candidate agents may comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, and derivatives, structural analogs or combinations thereof.

Assays of the present disclosure include controls, where suitable controls include a subject non-human animal model that has been exposed to activating light, but has not been administered the test agent.

A subject screening method can be used to analyze the effect of a test agent on any of a variety of adverse psychological and physiological states, including, but not limited to, dysphoria, depression, anhedonia, suicidality, agitation, anxiety, drug addiction withdrawal symptoms, and the like. In some cases, a test agent that reduces or alleviates an adverse state is considered a candidate agent for treating a mood disorder (e.g., major depression disorder (i.e., unipolar disorder), mania, dysphoria, bipolar disorder, dysthymia, cyclothymia, and the like). Thus, although depression is discussed in the present disclosure, a subject screening method can be used to analyze the effect of a test agent on any of a variety of adverse states; and test agents identified can be considered candidate agents for treating any of a variety of mood disorders and other adverse psychological and physiological states.

Symptoms of depression in the non-human animal model include, e.g., reduced escape-related behavior, anxiety, and stress. Tests for depression and/or anxiety and/or stress include the forced swim test (FST) (see, e.g., Porsolt et al. (1977) Nature 266:730; and Petit-Demouliere, et al. (2005) Psychopharmacology 177: 245); the tail suspension test (see, e.g., Cryan et al. (2005) Neurosci. Behav. Rev. 29:571; and Li et al. (2001) Neuropharmacol. 40:1028); conditioned place aversion (see, e.g., Bechtholt-Gompf et al. (2010) Neuropsychopharmacol. 35:2049); the novelty hypophagia test (Dulawa, et al. (2005) Neurosci. Biobehav. Rev. 29:771); the social defeat stress test (see, e.g., Blanchard et al. (2001) Physiol Behav. 73:261-271; and Kudryavtseva et al. (1991) Pharmacol. Biochem. Behav. 38: 315); the sucrose preference test (see, e.g., Kurre Nielsen, et al. (2000) Behavioural Brain Research 107:21-33); the open field test (see, e.g., Holmes (2001) Neurosci. Biobehav. Rev. 25:261-273); the elevated plus maze test (see, e.g., Holmes (2001) supra); and the like. Any such test can be used in a subject screening method.

### Methods of Identifying Agents Suitable for Treating Depression

The present disclosure provides methods of identifying candidate agents for treating depression. In some cases, the methods generally involve: a) contacting a subject non-human animal (e.g., a rodent, such as a rat or a mouse) that expresses a light-responsive opsin in ventral tegmental area (VTA) dopaminergic (DA) neurons with a test agent, and b) comparing the behavior of the rodent in a depression assay to the behavior of a control rodent that has not been contacted with the test agent. An anti-depressive behavior of the rodent contacted with the test agent indicates that the test agent is a candidate for treating depression.

### Hyperpolarizing Opsin Expressed in DA Neurons of the VTA

In some cases, the active optogenetic inhibitor of neuronal activity (light-responsive opsin) is a halorhodopsin (e.g., NpHR) that promotes hyperpolarization of the DA neurons when activated by light at or near the VTA. Hyperpolarization of the DA neurons inhibits activity of these neurons. The non-human animal model exhibits characteristics of depression when the light-responsive opsin is activated by light. A test agent is administered to the non-human animal model. When DA neurons of the VTA are exposed to light of a wavelength (e.g., amber light) that activates that light-responsive opsin, a test agent that is a candidate agent for treating depression will ameliorate at least one symptom of depression in the non-human animal model.

Thus, in some cases, a subject method involves: a) contacting a subject non-human animal (e.g., a rodent, such as a rat or a mouse) that expresses a halorhodopsin (e.g., NpHR) that promotes hyperpolarization of the DA neurons when activated by light at or near the VTA with a test agent, and b) determining the effect of the test agent on the behavior of the rodent in a depression assay. An anti-depressive behavior of the rodent contacted with the test agent, compared to the behavior of a control rodent that has not been contacted with the test agent, indicates that the test agent is a candidate for treating depression. In these embodiments, the determining step is carried out after, or concurrently with, exposure of the halorhodopsin to light of a wavelength that would activate the halorhodopsin.

The present disclosure provides a method for identifying a candidate agent for treating an adverse psychological or physiological state in an individual, where the method generally involves: contacting a rodent that expresses an active optogenetic inhibitor of neuronal activity in VTA dopaminergic neurons with a test agent, and determining the effect of the test agent on a behavior of the rodent in a conditioned place aversion (CPA) test. Modulation of the CPA response behavior of the rodent contacted with the test agent, compared to the behavior of a control rodent that has not been contacted with the test agent, indicates that the test agent is a candidate agent for treating an adverse psychological state in an individual. In these embodiments, the determining step is carried out after, or concurrently with, exposure of the halorhodopsin to light of a wavelength that would activate the halorhodopsin. Advserse psychological and physiological states include, but are not limited to, dysphoria, depression, anhedonia, suicidality, agitation, anxiety, drug addiction withdrawal symptoms, and the like.

In some embodiments, the halorhodopsin is comprises both ER export and membrane trafficking signals. For example, in some cases, the halorhodopsin is an NpHR opsin protein that comprises, from the N-terminus to the C-terminus, the amino acid sequence at least 95% identical to the sequence shown in SEQ ID NO:1, the ER export signal, and the membrane trafficking signal. In other cases, the halorhodopsin is an NpHR opsin protein that comprises, from the N-terminus to the C-terminus, the amino acid sequence at least 95% identical to the sequence shown in SEQ ID NO:1, the membrane trafficking signal, and the ER export signal. In some cases, the membrane trafficking signal is derived from the amino acid sequence of the human inward rectifier potassium channel Kir2.1. In some cases, the membrane trafficking signal comprises the amino acid sequence KSRITSEGEYIPLDQIDINV (SEQ ID NO:17). In some cases, the ER export signal comprises the sequence FCYENEV (SEQ ID NO:13).

In some cases, the halorhodopsin-encoding nucleotide sequence is operably linked to a neuron-specific promoter, e.g., a promoter that provides for expression of the halorhodopsin in a neuron. In some embodiments, the promoter is a tyrosine hydroxylase promoter.

Symptoms of depression in the non-human animal model include, e.g., reduced escape-related behavior. A test agent of interest (e.g., a test agent that is a candidate agent for treating depression), increases escape-related behavior, compared to a control animal not treated with the test agent. For example, in some cases, a test agent of interest (e.g., a test agent that is a candidate agent for treating depression), increases escape-related behavior by at least about 10%, at least about 20%, at least about 30%, at least about 40%, at least about 50%, at least about 2-fold, or more than 2-fold, compared to a control animal not treated with the test agent.

Tests for depression include the forced swim test (FST) (see, e.g., Porsolt et al. (1977) Nature 266:730); the tail suspension test (see, e.g., Cryan et al. (2005) Neurosci. Behav. Rev. 29:571); and the like.

In some embodiments, a test agent increases performance in the tail suspension test. The tail suspension test is based on the fact that animals subjected to the short-term, inescapable stress of being suspended by their tail, will develop an immobile posture. A test agent that is a candidate agent for treating depression will reduce the immobility and promote the occurrence of escape-related behavior, compared to a control animal not treated with the test agent.

### Depolarizing Opsin in DA Neurons of the VTA

In some cases, the active optogenetic inhibitor of neuronal activity (light-responsive opsin) is a channelrhodopsin (e.g., ChR2) that promotes depolarization of DA neurons of the VTA when activated by light at or near the VTA. Depolarization of the DA neurons activates these neurons. The non-human animal model exhibits characteristics of depression under conditions of chronic mild stress (CMS) when the light-responsive opsin is not activated by light. Activation of the channelrhodopsin by light of an activating alleviates the symptoms of depression. A test agent is administered to the non-human animal model. When DA neurons of the VTA are not exposed to light of a wavelength that activates the depolarizing light-responsive opsin, a test agent that is a candidate agent for treating depression will ameliorate at least one symptom of depression in the non-human animal model. In some cases, when DA neurons of the VTA are not exposed to light of a wavelength that activates the depolarizing light-responsive opsin, a test agent that is a candidate agent for treating depression will ameliorate at least one symptom of depression in the non-human animal model to the same extent as exposure to light of an activating wavelength.

Thus, in some cases, a subject method involves: a) contacting a subject non-human animal (e.g., a rodent, such as a rat or a mouse) that expresses a channelrhodopsin (e.g., ChR2) that promotes depolarization of the DA neurons when activated by light at or near the VTA with a test agent, and b) determining the effect of the test agent on the behavior of the rodent in a depression assay. An anti-depressive behavior of the rodent contacted with the test agent, compared to the behavior of a control rodent that has not been contacted with the test agent, indicates that the test agent is a candidate for treating depression. In these embodiments, the determining step is carried out in the absence of exposure of the channelrhodopsin to light of a wavelength that would activate the channelrhodopsin. In some cases, a test agent that is a candidate agent for treating depression alleviates one or more symptom of depression to an extent that is at least about 50%, at least about 60%, at least about 70%, at least about 80%, at least about 90%, or more than 90%, of the extent to which the symptom of depression is alleviated by exposure of the channelrhodopsin to light of a wavelength that would activate the channelrhodopsin. In some cases, a test agent that is a candidate agent for treating depression alleviates one or more symptom of depression to the same extent as exposure of the channelrhodopsin to light of a wavelength that would activate the channelrhodopsin.

CMS conditions have been described in the art. See, e.g., Forbes et al. (1996) Physiol. & Behavior 60:1481; and are described in the Examples.

In some cases, the depolarizing opsin is a light-responsive cation channel protein comprising an amino acid sequence at least about 90%, 91%, 92%, 93%, 94%, 95%, 96%, 97%, 98%, 99%, or 100% identical to the sequence set forth in SEQ ID NO:5. In some embodiments, the light-responsive channel protein comprises a membrane trafficking signal and/or an ER export signal. In some cases, the membrane trafficking signal comprises the amino acid sequence KSRITSEGEYIPLDQIDINV (SEQ ID NO:17). In some cases, the ER export signal comprises the sequence FCYENEV (SEQ ID NO:13).

### Methods of Identifying Targets for Therapeutic Intervention

A subject non-human animal model can be used to identify additional targets for therapeutic intervention in the treatment of depression. Thus, the present disclosure provides a method of identifying a protein that promotes depression, where such a protein would be considered a potential therapeutic target for treating depression, e.g., a target that can be used to identify drugs that modulate activity of the target and thereby treat depression.

In some cases, the present disclosure provides a method for identifying a protein that promotes depression in an individual, where the method generally involves: a) contacting a subject non-human animal that expresses an active optogenetic activator of neuronal activity in medial prefrontal cortex (mPFC) excitatory neurons with the protein, and b) comparing the behavior of the non-human animal in a depression assay to the behavior of a control rodent that has not been contacted with the protein. A depressive behavior of the non-human animal contacted with the agent indicates that the protein promotes depression.

A subject non-human animal can be contacted with a protein either by introducing the protein itself into the animal or by introducing into the animal a nucleic acid comprising a nucleotide sequence encoding the protein. For example, an expression construct comprising a nucleotide sequence encoding a protein to be tested for a depression-inducing effect can be introduced directly into a neuron (e.g., by injection, as described above). A cDNA library can be tested in this manner.

In some cases, the active optogenetic activator is a ChR2. In some cases, the non-human animal that expresses an active optogenetic activator of neuronal activity in mPFC excitatory neurons is engineered by: expressing an optogenetic activator of neuronal activity in mPFC excitatory neurons, and exposing the dorsal raphe nucleus (DRN) to light to activate the optogenetic activator.

In some cases, the active optogenetic activator of neuronal activity in mPFC excitatory neurons is a light-responsive cation channel protein comprising an amino acid sequence at least about 90%, 91%, 92%, 93%, 94%, 95%, 96%, 97%, 98%, 99%, or 100% identical to the sequence set forth in SEQ ID NO:5. In some embodiments, the active optogenetic activator of neuronal activity in mPFC excitatory neurons comprises a membrane trafficking signal and/or an ER export signal. In some cases, the membrane trafficking signal comprises the amino acid sequence KSRITSEGEYIPLDQIDINV (SEQ ID NO:17). In some cases, the ER export signal comprises the sequence FCYENEV (SEQ ID NO:13).

Methods of Evaluating Drugs in Development for Treating Disorders Other than Depression

A subject non-human animal model can be used to test a drug, which is being developed for treatment of a disorder other than depression, for a depression-inducing effect. A drug that induces symptom(s) of depression in a subject non-human animal model may need to be re-evaluated for its suitability in treating the disorder other than depression; may need to be chemically modified so that it no longer induces symptom(s) of depression in a subject non-human animal model, yet retains efficacy in treating the disorder other than depression; or may need to include in a warning label the possibility that the drug may possibly induce symptom(s) of depression.

In some cases, the present disclosure provides a method for screening an agent (e.g., a drug under development for treating a disorder other than depression) for the ability to promote depression in an individual, where the method generally involves: a) contacting a subject non-human animal that expresses an active optogenetic activator of neuronal activity in medial prefrontal cortex (mPFC) excitatory neurons with the agent, and b) comparing the behavior of the non-human animal in a depression assay to the behavior of a control rodent that has not been contacted with the agent. A depressive behavior of the non-human animal contacted with the agent indicates that the agent promotes depression. In some cases, the active optogenetic activator is a ChR2. In some cases, the non-human animal that expresses an active optogenetic activator of neuronal activity in mPFC excitatory neurons is engineered by: expressing an optogenetic activator of neuronal activity in mPFC excitatory neurons, and exposing the dorsal raphe nucleus (DRN) to light to activate the optogenetic activator.

In some cases, the active optogenetic activator of neuronal activity in mPFC excitatory neurons is a light-responsive cation channel protein comprising an amino acid sequence at least about 90%, 91%, 92%, 93%, 94%, 95%, 96%, 97%, 98%, 99%, or 100% identical to the sequence set forth in SEQ ID NO:5. In some embodiments, the active optogenetic activator of neuronal activity in mPFC excitatory neurons comprises a membrane trafficking signal and/or an ER export signal. In some cases, the membrane trafficking signal comprises the amino acid sequence KSRITSEGEYIPLDQIDINV (SEQ ID NO:17). In some cases, the ER export signal comprises the sequence FCYENEV (SEQ ID NO:13).

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

### Example 1

### Dopamine Neurons Modulate Both Neural Encoding and Expression of Depression-Related Behavior

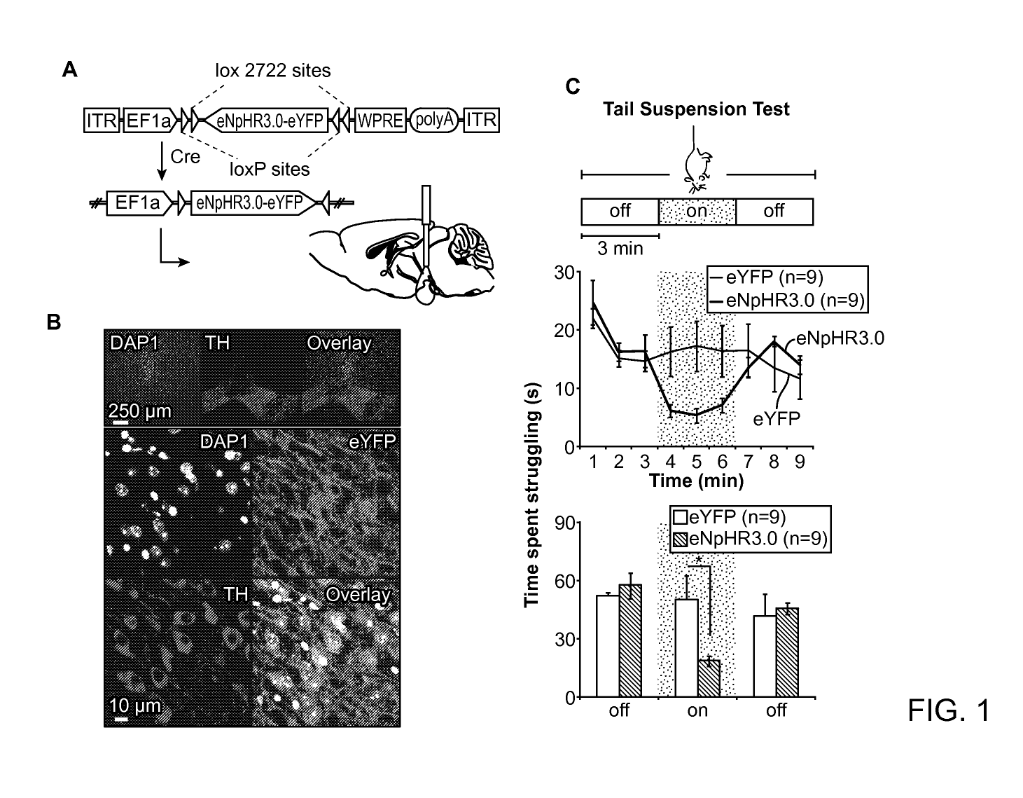

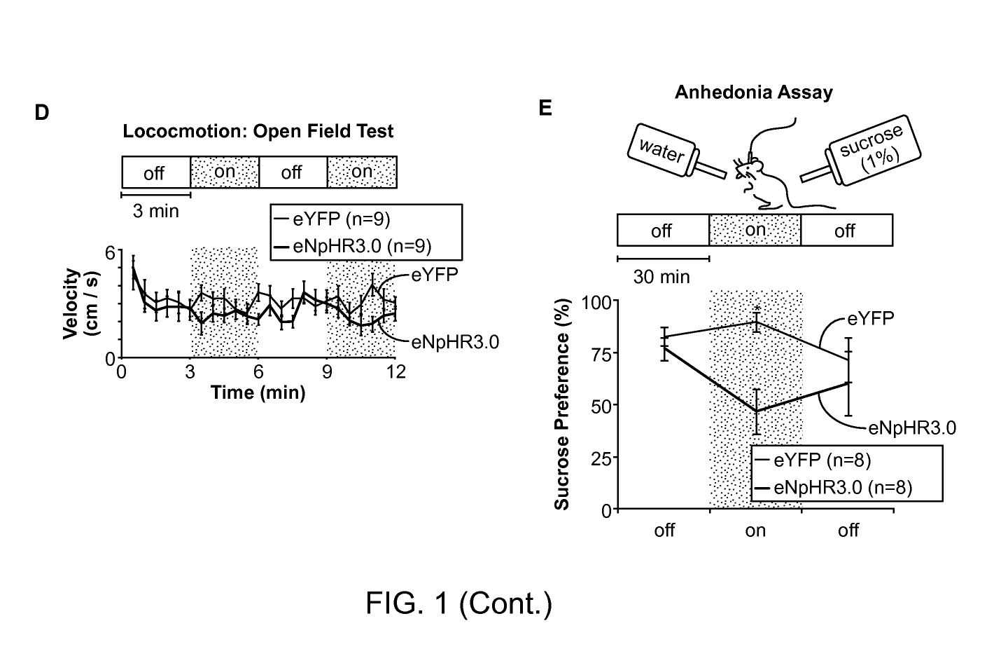

Whether the activity of ventral tegmental area (VTA) dopamine neurons was required for maintaining normal levels of motivation and hedonia was investigated. To test whether selectively inhibiting the activity of VTA DA neurons could acutely induce depression-like behavior, cell-type specific targeting and precise temporal manipulations allowed by optogenetic techniques (21-23) were utilized. To inhibit VTA DA neurons, expressed an enhanced halorhodopsin that hyperpolarizes neuronal membranes upon illumination with amber light (eNpHR3.0) (23) was selectively expressed in tyrosine hydroxylase (TH) positive neurons by injecting the double-floxed cre-dependent viral construct into the VTA of TH::Cre mice (24) (FIG. 1A) before testing these animals on multiple depression assays.

Since eNpHR3.0 is fused to the enhanced yellow fluorescent protein (eYFP), the specificity of the targeting in the animals used for these behavioral assays was immunohistochemically verified by quantifying the proportion of VTA neurons that expressed eNpHR3.0-eYFP, or in age-matched controls expressing eYFP alone, that were TH+ (FIG. 1B. The two primary categories of depression assays in rodents involve measures of motivation (25-28) and anhedonia (27,29,30). These assays have been well-validated in that performance improves with chronic treatment of anti-depressant medications (25-27,29,30).

In the context of depressive phenotypes, motivation is assayed by presenting the rodent with an inescapable stressor, such as suspension by the animal's tail or a forced swim in cold water. Assay analysis entails quantifying the proportion of time that the animal spends performing escape-related behavior, or struggling, relative to the time spent immobile. Immobility, hanging in the tail suspension test (TST) or floating in the forced swim test (FST) in such assays has historically been interpreted as a sign of “behavioral despair” (27,28). Here, when mice with VTA DA neurons expressing eNpHR3.0 were compared to eYFP controls on a 9-min session on the TST with 3 epochs including a baseline light off epoch, a light on epoch and then another light off epoch, we saw a significant reduction in escape-related behavior upon constant illumination with 591 nm light (2-way ANOVA revealed a GroupxEpoch Interaction, F4,38=3.95, p=0.00089, with a Bonferroni post-hoc test showing a significant reduction in escape-related behavior in the eNpHR3.0 group relative to the eYFP group, p<0.05) that returned to baseline once the light was off (FIG. 1C).

Whether this transient reduction in escape-related behavior was a gross locomotor effect, rather than an increase in motivation, was investigated by evaluating these same animals while freely-exploring in a novel, but not stressful environment, on the open field test (OFT) during a 12-min session across two 3-min alterations between light off and light on conditions. While there may have been a subtle trend toward decreased locomotion in the eNpHR3.0 group upon illumination, there was no significant difference in locomotor velocity between groups (2-way ANOVA showed no GroupxEpoch interaction, F3,48=1.76, p=0.17). The significant reduction in escape-related behavior and the non-significant trend towards reduced locomotion may reflect a reduction in motivation upon inhibition of VTA DA neurons.

In addition to assaying escape-related behavior in the face of an inescapable stressor, a novel variation on the well-validated anhedonia assay, the sucrose preference test (29-34), was developed to determine whether selective inhibition of VTA DA neurons could acutely induce anhedonia. To increase the sensitivity of our sucrose preference measure, the number of licks on spouts delivering either water or a 1% sucrose solution during a 90-min session was quantified by automated detection, to determine sucrose preference within a baseline 30-min light off epoch, followed by a 30-min light on epoch, ending with another 30-min light off epoch (FIG. 1E). Remarkably, a significant reduction in sucrose preference during illumination in eNpHR3.0, but not eYFP control, mice was observed (FIG. 1E; 2-way ANOVA revealed a significant effect of Opsin, F1,42=6.31, p=0.016; Bonferroni post-hoc tests revealed a significant difference between groups only in the light on epoch, p<0.05). Thus, inhibition of VTA DA neurons significantly reduced escape-related behavior in the face of an inescapable stressor, as well as acutely eliciting anhedonia, as measured by reduced sucrose preference. Taken together, it is shown here that selective inhibition of VTA DA neurons acutely mimics a depression-like phenotype in measures of both increased “behavioral despair” and anhedonia (27,28).

Next, whether phasic activation of VTA DA neurons could serve to rescue a depression-like phenotype induced by unpredictable chronic mild stress (CMS) (31,32,34,35) was investigated. In humans, most patients suffering from depression state that chronic stress gradually triggered a long-lasting depressive state (on the order of months), rather than a brief (on the order of days) set of stressful events (36-40, 19, 41). To faithfully model a depression-like state as observed in humans, an unpredictable Chronic Mild Stress (CMS) paradigm was used to induce a depressive-like state in rodents (32, 34-36, 42-47), wherein unpredictable mild stressors were delivered twice daily for 8-12 weeks in adult rodents. CMS has been shown to produce decreases in motivation, as assayed by a reduction in escape-related behavior in the face of inescapable stressors, as well as anhedonia, as measured by sucrose preference (27,29-32,34,44,18).

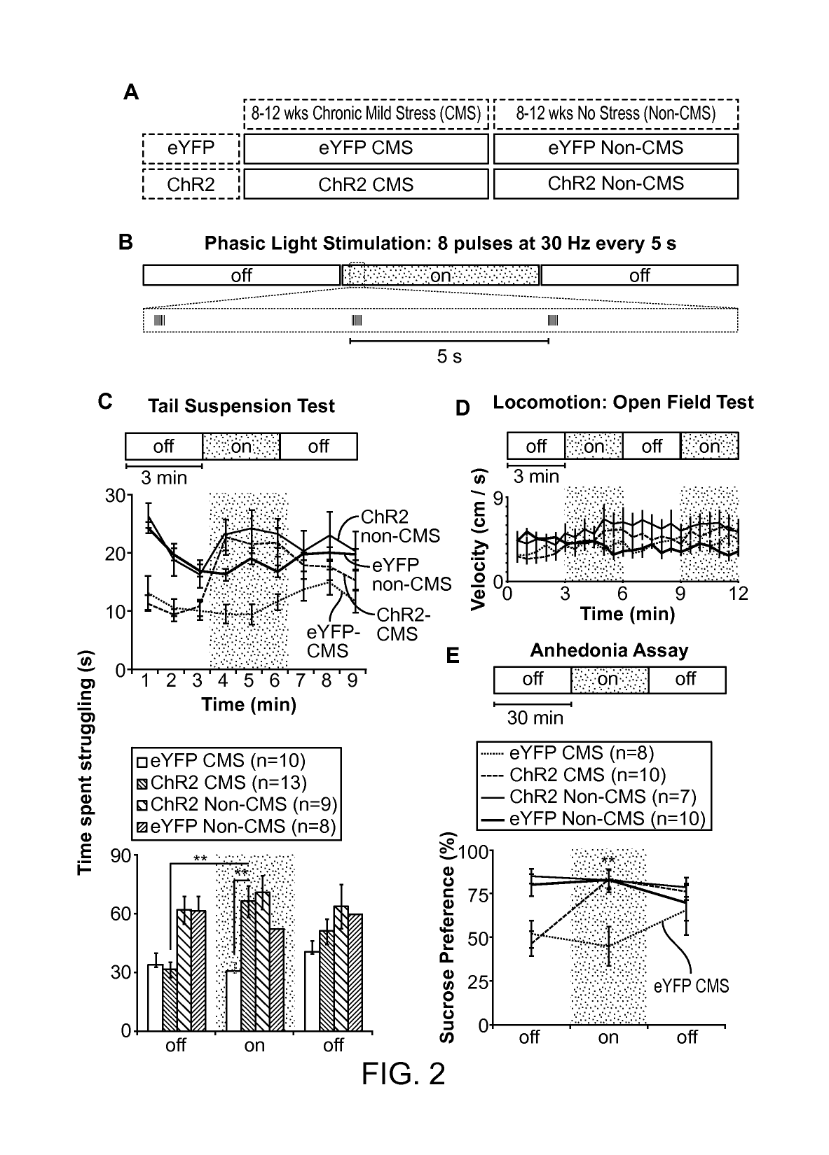

Since it was demonstrated that inhibiting VTA DA neurons acutely produced a depression-like phenotype (FIG. 1), it was then determined whether activation of VTA DA neurons could rescue a depression-like phenotype induced by chronic stress. To selectively activate VTA DA neurons, viral transduction methods were used to selectively express channelrhodopsin (ChR2), a light-activated cation channel that depolarizes membranes and produces action potentials with millisecond precision (22,48) and has been shown to release dopamine transients in the nucleus accumbens (NAc) when expressed in VTA TH+ at the parameters used (24,49,50). To test this, four experimental groups were included (FIG. 2A): 1) A group with ChR2-transduced TH+ neurons in the VTA, that was exposed to a chronic mild stress (CMS) protocol for 8-12 weeks, 2) As a control, the eYFP fluorophore was expressed alone in VTA DA neurons; these animals were treated with the CMS protocol, 3) Expressed either ChR2 or 4) eYFP alone in VTA DA neurons in animals that were housed in a low-stress environment (Non-CMS) for the 8-12 week duration.

To test whether phasic firing in VTA DA neurons could rescue a CMS-induced depression-like phenotype, we examined animals during baseline (off), phasic illumination (on) and post-illumination (light off) epochs (FIG. 2B) during multiple depression assays. To produce phasic firing in VTA DA neurons, a sparse, bursting illumination pattern was used (FIG. 2B; 8 pulses at 30 Hz, 5-ms pulse width, every 5 seconds) to elicit phasic spiking24 and the release of dopamine transients (24,49) during illumination (on) epochs. To examine the effects of phasic firing in VTA DA neurons on motivation all 4 groups of animals were subjected to the Tail Suspension Test (TST) and the amount of struggling or escape-related behavior during each epoch was quantified.

At baseline, consistent with previous studies (25,32,33,43), it was observed that CMS reduced the amount of struggling relative to Non-CMS controls by ˜50% (eYFP CMS=33.8±6.1; ChR2 CMS=31.3±3.9; ChR2 Non-CMS=61.7±7.3; eYFP Non-CMS 61.3±7.6; FIG. 2C). A 2-way ANOVA not only revealed a significant GroupxEpoch interaction, F6,108=3.36, p=0.0045, but also a very strong effect of Group, F3,108=16.92, p<0.0001. Upon illumination, the ChR2 CMS group showed a significant increase in escape-related behavior, relative to the eYFP CMS group (p<0.001, Bonferroni post-hoc test). Thus, phasic illumination of VTA DA neurons in ChR2 CMS mice, but not eYFP CMS mice, rescued the CMS-induced depression-like phenotype on the order of seconds (FIG. 2C). Importantly, a significant difference in struggling between eYFP Non-CMS and ChR2 Non-CMS mice upon illumination (Bonferroni post-hoc test) was not observed, indicating that the increase in escape-related behavior was specific to animals that displayed a stress-induced depression-like phenotype.

Since the DA system is also linked to locomotion, whether the stimulation parameters that were used during the TST were inducing gross locomotor effects was tested. The locomotion of all the mice included in the TST assay was examined in an open field chamber during two 3-min light on epochs, interleaved with two 3-min light off epochs using the same phasic illumination parameters described above (FIG. 2D). Although there was a trend toward increased velocity in ChR2 groups upon illumination, a significant GroupxEpoch interaction in a 2-way ANOVA was not observed (F9,152=0.99, p=0.4493), and no detectable differences were revealed by Bonferroni post-hoc tests. However, a significant effect of Group was observed (F3,152=5.06, p=0.0023), which may reflect differences between CMS and Non-CMS groups in initial exploration in the open field chamber (FIG. 2D).

Next, it was asked whether phasic activation of VTA DA neurons would also rescue CMS-induced decreases in sucrose preference. To detect an acute change in sucrose preference, a novel variation on the sucrose preference test was developed to increase the sensitivity of this assay. Using a lickometer to detect licks at spouts delivering either water or 1% sucrose solution, sucrose preference was assayed in a single 90-min session, across three 30-min epochs (FIG. 2E). A 2-way ANOVA revealed a significant GroupxEpoch interaction (F6,62=4.33, p=0.001), as well as a significant effect of Group (F3,31=3.40, p=0.0299). Consistent with previous studies, baseline measurements showed that eYFP and ChR2 CMS mice had a significantly lower sucrose preference in comparison to eYFP and ChR2 Non-CMS mice prior to illumination on the lickometer assay (Bonferroni post-hoc tests, p<0.05 and p<0.01, respectively). However, phasic activation of VTA DA neurons acutely rescued the CMS-induced anhedonic effect in ChR2 CMS, but not eYFP CMS, animals (1-way ANOVA, Dunn's post-hoc test comparing baseline to light on epoch, p<0.01 for ChR2 CMS mice, p=0.2851 for eYFP CMS mice).

The data presented above demonstrate that there is a bidirectional effect of VTA DA neuron activity on multiple assays for depression-related behaviors. However, VTA DA neurons project to multiple regions throughout the brain (51-54), so it is not clear which downstream targets may be contributing to this behavior. Furthermore, there is evidence that VTA DA neurons co-release glutamate in the ventral striatum (55-57). Given that deep brain stimulation in the ventral striatum, particularly the nucleus accumbens (NAc) in human patients diagnosed with major depressive disorder has helped to alleviate the symptoms of depression (58,59), it was tested whether glutamate or dopamine transmission in the NAc was mediating the light-induced rescue of this depression-like phenotype in mice.

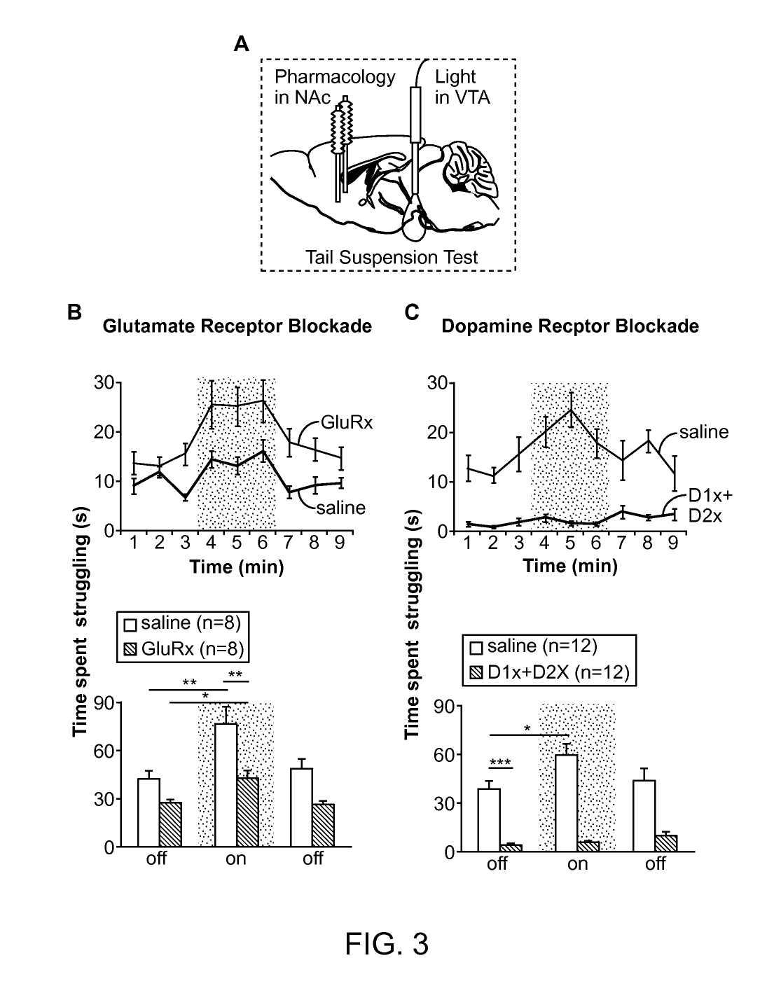

To investigate the contribution of VTA DA neuron transmission in the NAc during the TST, an additional group of mice was included, which were implanted with bilateral guide cannulae in the NAc in addition to viral transduction of VTA DA neurons and chronic implantation of a fiber optic cable aimed at the VTA (FIG. 3A), prior to undergoing 8-12 weeks of unpredictable chronic mild stress. To investigate the functional role of glutamate transmission upon the phasic activation of VTA DA neurons, a within-subject comparison was performed, counterbalanced for order, and infused either saline or a mixture of the α-amino-3-hydroxy-5-methyl-4-isoxazolepropionic acid receptor (AMPAR) antagonist, 2,3-dihydroxy-6-nitro-7-sulfamoyl-benzo[f]quinoxaline-2,3-dione (NBQX), and N-Methyl-D-aspartate receptor (NMDAR) antagonist, (2R)-amino-5-phosphonovaleric acid (AP5), into the NAc just prior to testing on the TST.

A 2-way ANOVA revealed a significant GroupxEpoch interaction (F2,28=3.69, p=0.0379), a significant effect of Group (F1,14=7.24, p=0.0176), and a significant effect of Epoch (F2,28=38.98, p<0.0001). Following intra-NAc saline treatment, we replicated the rescue of the stress-induced depression-like phenotype upon VTA illumination (1-way ANOVA, p<0.001, Dunn post-hoc test comparing baseline to light on epochs, p<0.01; FIG. 3B). Following intra-NAc glutamate receptor blockade, a rescue of the stress-induced depression-like phenotype upon illumination was also observed (1-way ANOVA, p<0.001, Dunn post-hoc test comparing baseline to light on epochs, p<0.001; FIG. 3B). Indeed, the amount of time spent struggling was greater overall in the animals treated with glutamate receptor antagonists, as seen by the significant effect of Group.

Since the NAc receives robust glutamatergic innervation from a number of regions, including the PFC, Amygdala and Hippocampus, it was speculated that the net effect of these inputs may serve to suppress escape-related behavior on the TST. Moreover, the findings presented herein are consistent with recent studies showing that ketamine, an NMDAR antagonist, can acutely improve depression-like symptoms in humans (60-62).

Since it was demonstrated that glutamate transmission in the NAc was not required for mediating our light-induced increase on escape-related behavior during tail suspension, it was then tested whether dopamine signaling in the NAc was critically involved in modulating escape-related behavior. To do this, a within-subjects comparison was performed following intra-NAc saline or intra-NAc dopamine receptor antagonism, using a mixture of SCH 23390 (a D1 receptor antagonist) or raclopride (a D2 receptor antagonist) prior to testing on the TST. A significant attenuation of escape-related behavior was observed with NAc dopamine receptor blockade during both baseline and illumination epochs (2-way ANOVA revealed a significant DrugxEpoch interaction, F2,44=3.52, p=0.0381, a significant effect of Drug, F1,22=53.74, p<0.0001, and a significant effect of Epoch, F2,44=3.48, p=0.0395; FIG. 3C).

These findings are consistent with the hypothesis that dopaminergic innervation from the VTA to the NAc is important for maintaining baseline levels of escape-related behavior as well as the rescue of a CMS-induced reduction of escape-related behavior upon increasing phasic activity of VTA DA neurons. The data are also consistent with reports that depletion of dopamine signaling produces depression-like symptoms in pre-Parkinsonian patients, who often experience depression prior to the onset of Parkinsonian symptoms.

The data above have demonstrated that selective inhibition of VTA DA neurons acutely produces depression-related behavior in measures of both motivation and anhedonia, and that phasic activation of VTA DA neurons acutely rescues an unpredictable chronic mild stress-induced depression-like phenotype, mediated by dopaminergic signaling in the NAc. However, dopamine receptor blockade in the NAc attenuated both light-induced and baseline levels of escape-related behavior, making it difficult to interpret the role of dopamine signaling in the NAc in mediating the light-induced rescue of depression-related behavior on this motivation assay. Therefore, the activity of NAc neurons during a depression assay under both baseline conditions and during phasic activation of VTA DA neurons was investigated. To do this, several new tools were combined.

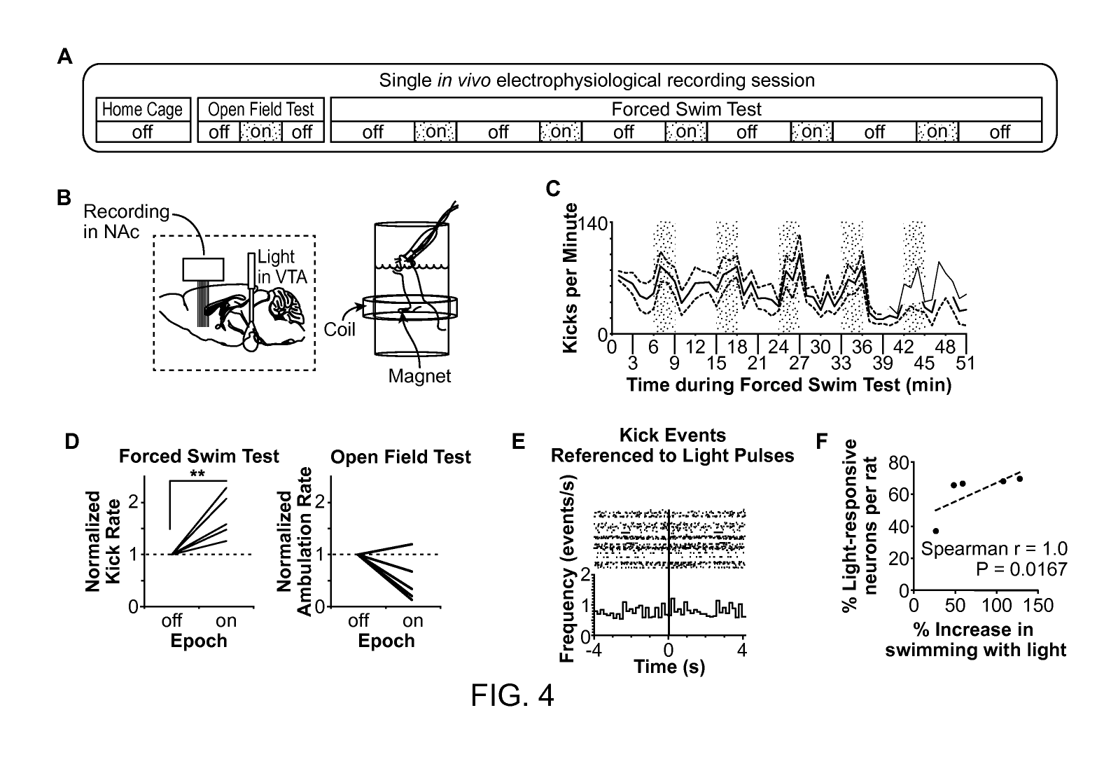

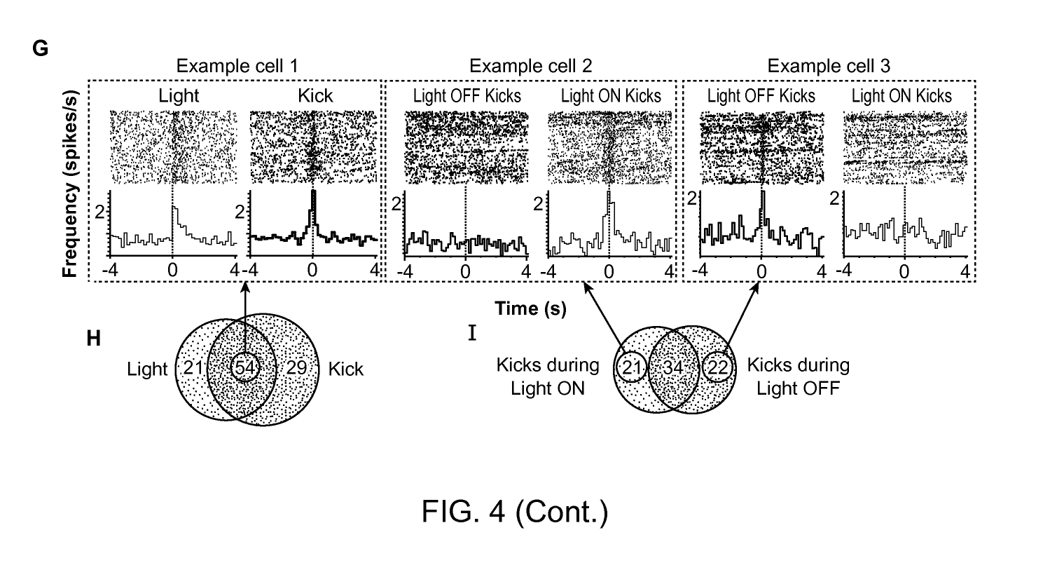

The first in vivo electrophysiological recordings were performed in the recently developed TH::Cre rat (50) during baseline activity in the home cage, exploration in the novel environment of the open field test, and the forced swim test (FIG. 4A) while intermittently illuminating ChR2-expressing VTA DA neurons following CMS (FIG. 4B). Since quantification of the forced swim test (FST) has traditionally been low-resolution measurement of epochs of immobility27, we needed to develop a novel method for millisecond-precision temporal resolution detection of escape-related behavior on the FST. To do this, a novel method of magnetic induction was utilized, using a magnetic coil on the outside of the forced swim tank, and a small magnet attached to the rat's foot to measure swimming kicks or escape-related behavior, and waterproofing the in vivo electrophysiological recording headstage (FIG. 4B).

Similar to the findings on the TST in mice, it was found the illumination of ChR2-expressing VTA DA neurons in 5 CMS TH::Cre rats increased escape-related behavior on the FST (Paired t-test, p=0.0088; FIGS. 4C and D), but there was no detectable effect of light on locomotion in the OFT (FIG. 4D). Although there was a substantial proportion of neurons that encoded both light and kick, light pulses and kick events were not time-locked on the order of seconds, as exemplified by the peri-event raster histogram of kicks referenced to light pulse trains, delivered in 30 Hz, 8 pulse trains every 5 seconds (FIG. 4E). Since we observe a robust increase in kick frequency during light on epochs relative to light off epochs, but do not observe time-locked kicking behavior to laser pulses, we speculate that that escape-related behavior is modulated by overall dopaminergic tone rather than isolated dopaminergic transients.

We then investigated the relationship between the increase in escape-related behavior in light on epochs relative to light off epochs and the proportion of all neurons recorded per animal that encoded phasic VTA DA neuron activation. We found a significant correlation (p=0.0167), using Spearman's correlation test, as the more NAc neurons that showed phasic responses to VTA DA neuron activation per rat, the greater the relative increase in escape-related behavior upon VTA DA neuron activation (FIG. 4F). This could be due to anatomical variation of individual animals as well as experimental variations such as opsin expression. We recorded a total of 123 NAc neurons in 5 CMS TH::Cre rats across the entire session and examined the proportion of neurons that showed phasic responses to light pulses delivered to the VTA, or kicks in the FST.

We found that 75 of 123 (61%; FIGS. 4G and 4H) NAc neurons showed phasic responses to light: 15 of these 75 neurons showed phasic inhibitions to light (20% of light-responsive neurons), while 60 of 75 responses were phasic excitations (80% of light-responsive neurons). 83 of 123 NAc neurons (67%; FIGS. 4G and 4H) encoded escape-related behavior, as seen by phasic responses to kick events: 14 of these neurons (17%) showed phasic inhibitions upon kick and 69 of these neurons (83%) showed phasic excitations in response to kick. 54 neurons encoded both light pulses and kick events, as seen in the representative peri-event raster histogram (FIGS. 4G and 4H).

To examine whether phasic firing of VTA DA neurons could modulate the encoding of escape-related behavior in the NAc, we separated kick events occurring in light on epochs from kick events occurring in light off epochs. While 34 of 123 neurons (28%) encoded the kick in both light on and light off epochs, we found that activation of VTA DA neurons modulated the encoding of kick events in two subpopulations of neurons (FIGS. 4G and 4I). 21 of 123 neurons (17%) selectively encoded escape-related behavior only during the light on epoch, and 22 of 123 neurons (18%) selectively encoded escape-related behavior only during the light off epoch (FIGS. 4G and 4I).

Next, we examined the firing rate dynamics across different epochs in the same session. While there was a relatively modest change in the distribution of firing rates across the population of NAc neurons across various epochs, the net change in distribution did not capture the individual neuronal changes in firing, as there were subpopulations of neurons that either increased or decreased firing rate across different epochs. Subpopulations of neurons showed firing rate changes when moved from the home cage to a novel environment, the open field chamber (33%; 18 increased and 22 decreased firing rate), and subpopulations of neurons showed firing rate changes upon illumination in the OFT (24%; 24 increased and 6 decreased firing rate) and FST (30%; 18 increased and 19 decreased firing rate). However, a much greater proportion of neurons showed changes in firing rate when the rat was moved from a novel environment, the open field chamber, to a stressful environment, the forced swim tank, regardless of whether the comparison was during a light off epoch (86%; 27 increased and 79 decreased firing rate) or during a light on epoch (75%; 19 increased and 73 decreased firing rate). To summarize our findings here, selective inhibition of VTA DA neurons acutely induces depression-related behaviors reflecting an increase in “behavioral despair” (63) and anhedonia.

The chronic presentation of unpredictable mild stressors induced a lasting depression-like phenotype, which was rescued by the phasic activation of VTA DA neurons. Dopamine, but not glutamate, receptor activation in the NAc is required for mediating escape-related behavior. NAc neurons encode the phasic activation of VTA DA neurons as well as escape-related behavior. Importantly, the encoding of escape-related behavior is modulated by VTA DA neuron activation. We also show that the majority of NAc neurons show significant changes in firing rate upon exposure to an inescapable stressor, and that more NAc neurons show decreases, rather than increases, in tonic firing rates.

Our findings are consistent with other in vivo electrophysiological recordings in depression models in rats, showing reduced bursting activity that was restored upon treatment with the antidepressant, desipramine (64). While there have been some reports that indicate that VTA DA neuron firing and bursting is increased in mice susceptible to a 10-day social defeat model of depression (65,66), the differences in these findings could stem from differences in the animal model, the duration of the paradigm, the specific recording sites within the midbrain, or other experimental differences. Our data also support existing models of dopamine function mediating motivation and hedonic responses in the context of addiction and reward-related behavior (11-13,15,20,67-69), as well as VTA dopamine firing underlying the anticipation or receipt of reward (70,71), or the experience of hedonic pleasure (24) or reward-seeking (48,49). Most importantly, our results may provide a mechanistic explanation for the antidepressant effects achieved by deep brain stimulation in the NAc (57,58). These studies, in parallel with antidepressant effects of deep brain stimulation in the subgenual cingulate cortex (71), suggest that a psychiatric disease defined by a constellation of different classes of symptoms may be mediated by multiple neural circuit pathologies. The complexity of mood disorders and the challenge of modeling psychiatric diseases in animals represent obstacles in pinpointing the precise neural dysfunctions mediating the symptoms of depression. However, the potential impact of identifying the circuit mechanisms that mediate even a subset of depression-related symptoms is profound. Studying circuits that are well-conserved between rodents and humans, such as the mesolimbic dopamine system, enhances the likelihood that anti-depressant manipulations in rodent models will aid the development of improved therapeutic interventions in humans.

FIG. 1. Selective inhibition of ventral tegmental area (VTA) dopamine neurons induces a depression-like phenotype. A, Schematic of Cre-dependent AAV. Upon delivery into TH::IRES-Cre transgenics, eNpHR3.0 will be selectively expressed in tyrosine hydroxylase-positive neurons. B, Confocal images of midbrain dopamine neurons; orange dotted rectangle indicates location of the optical fiber aimed to illuminated the VTA. Below, close-up images of the VTA neurons directly below the fiber track. C, Photoinhibition of VTA DA neurons acutely induces a reduction in escape-related behavior, *P<0.05. In FIG. 1C, the left-hand bars in each set are eYFP; the right-hand bars in each set are eNpHR3.0. D, Inhibition of VTA DA neurons does not produce a detectable difference in locomotion in the open field test. E, Schematic and results of 90-min anhedonia assay. Photoinhibition of VTA DA neurons induces an acute reduction in sucrose preference, *P<0.05.

FIG. 2. Sparse, phasic photoactivation of VTA DA neurons rescues a stress-induced depression-like phenotype. A, Diagram of the four experimental groups included in the experiment. B, Schematic of the illumination pattern, with 473 nm light, used to elicit phasic bursts of activity in ChR2-expressing VTA DA neurons. C, Phasic illumination of VTA DA neurons rescues a stress-induced reduction in struggling on the tail suspension test (TST) in ChR2 CMS mice, but not eYFP CMS mice, **P<0.001. In FIG. 2C, the bars in each “off” and “on” set are, from left to right: eYFP CMS; ChR2 CMS; ChR2 Non-CMS; and eYFP Non-CMS. D, The illumination parameters used on the TST did not produce a detectable change in locomotor activity in an open field chamber. E, Phasic activation of VTA DA neurons acutely rescued the stress-induced decrease in sucrose preference in ChR2 CMS, but not eYFP CMS, animals, **P<0.01 for ChR2 CMS mice.

FIG. 3. Dopamine, but not glutamate, receptor signaling is required for mediating escape-related behavior. A, Schematic representation of bilateral NAc pharmacological manipulation in combination with VTA DA neuron illumination in animals treated with CMS. B, Antagonism of AMPAR and NMDAR glutamate receptors (GluRx) in the NAc does not block the baseline levels of struggling nor the light-induced increase in escape-related behavior on the tail suspension test. In FIG. 3B, the left-hand bars in each “off” and “on” data set are GluRx; the right-hand bars in each data set are saline. C, Antagonism of D1 and D2 dopamine receptors (D1x+D2x) in the NAc attenuates escape-related behavior, ***P<0.0001. In FIG. 3C, the left-hand bars in each “off” and “on” data set are saline; the right-hand bars in each data set are D1x+D2x.

FIG. 4. Phasic activation of VTA dopamine neurons modulates NAc neural encoding of escape-related behavior in the TH::Cre rat. A, Schematic overview of the in vivo electrophysiological recording session. B, Integration of in vivo electrophysiological recordings in the NAc, illumination of ChR2-expressing VTA DA neurons, and precision measurement of swimming behavior in TH::Cre rats treated with CMS. C, Phasic illumination of ChR2-expressing VTA DA neurons increases the escape-related behavior of TH::Cre rats in the forced swim test. D, Phasic illumination of ChR2-expressing VTA DA neurons increases kick rate in the forced swim test, but not ambulation rate in the open field test. E, Peri-event raster histogram showing kick events referenced to the train of 8 light pulses, indicated by blue lines, shows that kick events are not time-locked to light pulses. F, Scatterplot showing a correlation between the degree to which each rat's swimming behavior was modulated by VTA DA neuron activation relative to the proportion of all neurons recorded from a given subject, P=0.0167. G, Peri-event raster histograms for representative neurons showing phasic excitation to both light pulses and kick events (Example cell 1), and for neurons that selectively encoded kick events during either the light on epoch (Example cell 2) or the light off epoch (Example cell 3). H, Population summary of neurons showing phasic responses to both light pulses and kick events showing that of 123 NAc neurons recorded from 5 CMS TH::Cre rats, phasic responses to VTA illumination were seen in 75 NAc neurons, and phasic responses to kicking behavior was seen in 83 NAc neurons, with 54 neurons showing phasic response to both light and kick events. I, Population summary of the proportion of neurons showing differential encoding of escape-related kick events during light on and light off epochs during the forced swim test. 55 of 123 NAc neurons responded to kicks during light on epochs, 56 of 123 showed phasic responses to kicks during light off epochs, and 34 of 123 responded to kicks during both light epochs. 21 NAc neurons selectively encoded kick events during light on epochs, while 22 of 123 neurons selectively encoded kick events during light off epochs.
1. Kessler et al. JAMA: the journal of the American Medical Association 289: 3095 (2003)
2. Kendler et al. The American journal of psychiatry (1998)
3. Kessler et al. Arch Gen Psychiatry 62: 617-627 (2005)
4. van Geffen et al. The Annals of Pharmacotherapy 42: 218-225 (2008)
5. Maddox et al. Journal of Psychopharmacology 8: 48-51 (1994)
6. Smits et al. Mol Psychiatry 9: 433-441 (2004)
7. Jick et al. The Journal of the American Medical Association 292: 338-343 (2004)
8. Hall. The Lancet 367: 1959-1962 (2006)
9. Martinez et al. BMJ 330: 389-0 (2005)
10. Jick et al. BMJ 310: 215-218 (1995)
11. Phillips et al. Pharmacology Biochemistry and Behavior 90: 236-249 (2008)
12. Wise. Nature Reviews Neuroscience 4: 483-494 (2004)
13. Koob et al. Molecular psychiatry 1: 186 (1996)
14. Willner et al. The mesolimbic dopamine system: from motivation to action (1991)
15. Ikemoto et al. Neuropharmacology 47 (Supplement 1): 190-201 (2004)
16. Nestler et al. Biological Psychiatry 59: 1151-1159 (2006)
17. Willner et al. European Neuropsychopharmacology 1: 295-296 (1991)
18. Muscat et al. Biological Psychiatry 28: 223-230 (1990)
19. Cabib et al. Psychopharmacology 128: 331-342 (1996)
20. Ikemoto et al. Brain Research Reviews 31: 6-41 (1999)
21. Zhang et al. Nature 446: 633-639 (2007)
22. Boyden et al. Nat Neurosci 8: 1263-1268 (2005)
23. Gradinaru et al. 141: 154-165 (2010)
24. Tsai et al. Science 324: 1080-1084 (2009)
25. Cryan et al. Neuroscience & Biobehavioral Reviews 29: 571-625 (2005)
26. Steru et al. Psychopharmacology 85: 367-370 (1985)
27. Porsolt et al. European Journal of Pharmacology 47: 379-391 (1978)
28. Porsolt et al. Nature 266: 730-732 (1977)
29. Willner et al. Neuroscience & Biobehavioral Reviews 16: 525-534 (1992)
30. Willner. Psychopharmacology 83: 1-16 (1984)
31. Papp et al. Psychopharmacology 104: 255-259 (1991)
32. Forbes et al. Physiology & behavior 60: 1481-1484 (1996)
33. Strekalova et al. Neuropsychopharmacology 29: 2007-2017 (2004)
34. Willner et al. Psychopharmacology 93: 358-364 (1987)
35. Rygula et al. Behavioural brain research 162: 127-134 (2005)
36. Jesberger et al. Biological Psychiatry 20: 764-784 (1985)
37. Cohen et al. Psychological bulletin 98: 310 (1985)
38. Beck et al. University of Pennsylvania Press (2009)
39. Johnson et al. Journal of Psychosomatic research 22: 205-208 (1978)
40. MRCP et al. Journal of Human Stress 2: 3-12 (1976)
41. Swaab et al. Ageing research reviews 4: 141-194 (2005)
42. Paul. Trends in Pharmacological Sciences 12: 131-136 (1991)
43. Paul. Pharmacology & Therapeutics 45: 425-455 (1990)
44. Moreau et al. European Neuropsychopharmacology 2: 43-49 (1992)
45. Willner, et al. Neuroscience & Biobehavioral Reviews 16: 525-534 (1992)
46. Muscat et al. Psychopharmacology 109: 433-438 (1992)
47. Anisman et al. Neuroscience & Biobehavioral Reviews 29: 525-546 (2005)
48. Nagel et al. Proceedings of the National Academy of Sciences 100: 13940-13945 (2003)
49. Adamantidis et al. The Journal of Neuroscience 31: 10829-10835 (2011)
50. Witten et al. Neuron 72: 721-733 (2011)
51. Borgkvist et al. Neuron 70: 803-805 (2011)
52. Lammel et al. Neuron 57: 760-773 (2008)
53. Lammel et al. Neuron 70: 855-862 (2011)
54. Fields et al. Annu. Rev. Neurosci. 30: 289-316 (2007)
55. Stuber et al. The Journal of Neuroscience 30: 8229-8233 (2010)
56. Tecuapetla et al. The Journal of Neuroscience 30: 7105-7110 (2010)
57. Hnasko et al. Neuron 65: 643-656 (2010)
58. Malone Jr. et al. Biological Psychiatry 65L 267-275 (2009)
59. Bewernick et al. Biological Psychiatry 67: 110-116 (2010)
60. Berman et al. Biological Psychiatry 47: 351-354 (2000)
61. Maeng et al. Biological Psychiatry 63: 349-352 (2008)
62. Zarate et al. Arch Gen Psychiatry 63: 856-864 (2006)
63. Porsolt et al. European Journal of Pharmacology 47: 379-391 (1978)
64. Friedman et al. Journal of Molecular Neuroscience 34: 201-209 (2008)
65. Cao et al. The Journal of Neuroscience 30: 16453-16458 (2010)
66. Krishnan et al. Cell 131: 391-404 (2007)
67. Depue et al. Behavioral and Brain Sciences 22: 491-517 (1999)
68. Fibiger et al. Comprehensive Physiology (1986)
69. Salamone et al. Behavioural Brain Research 61: 117-133 (1994)
70. Schultz et al. Science 275: 1593-1599 (1997)
71. Stuber et al. Science 321: 1690-1692 (2008)
72. Mayberg et al. Neuron 45: 651-660 (2005)

### Example 2

### The Role of a Prefrontal Cortex to Brainstem Neural Projection in Goal-Oriented Behavioral States

### Materials and Methods

Subjects.

Male Long-Evans rats weighing 200-225 grams (approximately 6-8 weeks) were obtained from Charles River. Rats were maintained on a standard 12 hour light-dark cycle and given food and water ad libitum. Rats were initially housed two per cage Animals implanted with tetrode microdrives or fixed wire arrays were housed individually after implantation to minimize damage to recording hardware. Animals implanted with only fiber optics continued to be housed two per cage after implantation. All procedures conformed to guidelines established by the National Institutes of Health and have been approved by the Stanford Institutional Animal Care and Use Committee.

Automated Forced Swim Test (FST)

A 9-inch diameter tank of water (Tap Plastics, Mountain View, Calif.) was surrounded by a 10-inch diameter coil constructed from 10 pounds of 26 gauge enamel-coated copper wire. A 2 g rare-earth magnet was placed on the rat's back foot with a comfortably snug rubber band. Magnet placement was performed immediately before behavioral testing and was tolerated well by awake rats. During the FST, movement of the magnet within the coil of wire during swimming was found to induce a robust current in the coil. Coil voltage was bandpass filtered between 1 and 300 Hz, digitized at 2 kHz, and recorded for later analysis using a Digital Lynx data acquisition system (Neuralynx, Bozeman, Mont.). Data was simultaneously collected from a reference coil, and referencing was performed offline to reduce line noise. The same system was used to measure locomotor activity in a familiar cage by placing the induction coil directly underneath the cage; a similar method has been used previously to measure Parkinsonian tremor in rats (1). Coil data and video data were collected for all experiments. Manual scoring for validation of the automatic method was performed blind to experimental condition and automatically scored immobility/kick frequency. During manual scoring visual observations were taken every 5 seconds, and immobile epochs were defined as either an absence of movement or the minimum necessary movement required to stay afloat.

Tetrode Microdrive and Fixed Wire Array Placement

For the data shown in FIG. 2, rats reached a minimum of 400 g before surgery. For the data shown in FIG. 4, rats were bilaterally injected with virus in the mPFC (described below) at 8-10 weeks, and virus was allowed to express for a minimum of four months before electrode implantation (rats typically reached weights >400 g). Rats were initially anesthetized with 5% isoflurane. The scalp was shaved and rats were placed in a stereotaxic frame with non-rupturing ear bars. A heating pad was used to prevent hypothermia. Isoflurane was delivered at 1-3% throughout surgery; this level was adjusted to maintain a constant surgical plane. Ophthalmic ointment was used to protect the eyes. Buprenorphine (0.05 mg/kg, subcutaneous) and enrofloxacin (5 mg/kg, subcutaneous) were given before the start of surgery. A mixture of 0.5% lidocaine and 0.25% bupivicaine (100 μL) was injected subdermally along the incision line. The scalp was disinfected with betadine and alcohol. A midline incision exposed the skull, which was thoroughly cleaned. 8-11 skull screws were implanted at the periphery of the exposed skull to ensure stable recordings, a 2-mm craniotomy was drilled over the right mPFC, and the dura mater was carefully resected. A 4-tetrode microdrive with tetrodes wound from 13 μm nichrome (Neuralynx, Bozeman, Mont.) or a 24-electrode fixed wire array with 50 μm stainless steel electrodes (NB Labs, Denison, Tex.) was implanted over the craniotomy (AP: 2.7 to 3.3 ML: 0.8 DV: 4.0) and secured with dental acrylic. For the data shown in FIG. 4, a fiber optic patch cord targeting the DRN was also implanted at this time (described below). The acrylic was shaped to make a thin neck between the skull and the electrode interface board in order to facilitate waterproofing. The skin was sutured closed and the rats were given carprofen (5 mg/kg, subcutaneous) and lactated ringer's solution (2.5 mL, subcutaneous) and recovered under a heat lamp. After implantation tetrodes were adjusted daily.

Freely Moving Neurophysiology

Rats were briefly anesthetized with isoflurane. The headstage and tether (Neuralynx, Bozeman, Mont.) were connected to the microdrive or fixed wire array and secured with thread to prevent detaching during “wet dog” shakes. To protect the electronics from water damage a latex condom with both ends cut off was secured around the headstage attachment point with tightly wound rubber bands. At this time the magnet was attached to the back foot for behavioral readout. The total time under isoflurane anesthesia was limited to less than 10 minutes, and rats were allowed to recover for at least 1 hour before the start of recordings. For the FIG. 4 recordings, the fiber optic cable was attached immediately before the FST in order to minimize breakage from twisting during rotation, and light power was checked immediately after recordings to confirm that the fiber optic was intact. Neural data was acquired with a 64 channel Digital Lynx data acquisition system (Neuralynx, Bozeman, Mont.). Spiking channels were first referenced to an electrode exhibiting no spiking activity to reduce behavioral noise. The signal was then bandpass filtered between 600 and 6000 Hz and digitized at 32 kHz. Induction coil data and video data were also recorded during all epochs in order to validate the use of the induction coil method for both the FST and familiar cage activity. Data was recorded for a variable number of epochs depending on the experiment. For FIG. 2 we recorded 15 minutes of data pre-FST in a familiar cage, 15 minutes during the FST, and 15 minutes in a familiar cage post-FST. For FIG. 4 we recorded 15 minutes pre-FST in a familiar cage, 20 minutes during the FST with stimulation (five two-minute no-stimulation epochs interleaved with five two-minute stimulation epochs), 15 minutes in a familiar cage post-FST, and 20 minutes in a familiar cage post-FST with stimulation (five two-minute no-stimulation epochs interleaved with five two-minute stimulation epochs). Rats were handled gently during transfer between the familiar cage and the swim tank to minimize neural drift. After recording was completed the waterproofing was removed and the rat was placed under a heat lamp for 10 minutes to dry. Before sacrifice for histology rats were deeply anesthetized and current passed through all electrodes (50 μA for 30 seconds) to make electrolytic lesions for anatomical localization.

Virus Construction and Packaging

Recombinant AAV vectors were serotyped with AAV5 coat proteins and packaged by the viral vector core at the University of North Carolina. Viral titers were 2×10¹² particles/mL and 3×10¹² particles/mL respectively for AAV5-CaMKIIα-hChR2(H134R)-EYFP and AAV5-CaMKIIα-EYFP. Maps are available online at www(dot)optogenetics(dot)org.

Stereotaxic Virus Injection and Optical Fiber Implantation

Rats were prepared for surgery and given analgesics and fluids as described above. A midline incision exposed the skull, and craniotomies were made bilaterally above the mPFC. Virus was injected with a 10 μL syringe and a 33 gauge beveled needle with the bevel facing anteriorly at 150 nL/min using an injection pump. Two 1 μL injections were delivered to each hemisphere at AP 2.2 mm, ML 0.5, DV 5.2 and AP 2.2, ML 0.5, DV 4.2 for a total of 4 μL per rat. After each injection the needle was left in place for 7 minutes and then slowly withdrawn. The skin was sutured closed. Virus was allowed to express for a minimum of 4 months in order to allow time for sufficient opsin accumulation in the axons. At least 10 days before behavioral testing a fiber optic with an external metal ferrule (200 μm diameter, 0.22 NA, Doric Lenses, Québec, Canada) was implanted over the target structure of interest, as described previously2. Coordinates for mPFC implantation were AP 2.7, ML 0.5, DV 3.8. DRN fibers were implanted at a 30 angle from the right to avoid both the central sinus and the cerebral aqueduct, and the coordinates for the tip of the fiber were AP −7.8, ML 0.5, DV 5.9. The rats were prepared for surgery and given analgesics and fluids as described above. A midline incision was made, the skull was thoroughly cleaned, and a craniotomy was made over the mPFC or the DRN. Four skull screws were attached, and the fiber optic was lowered over the mPFC or the DRN. A thin layer of metabond was used to firmly attach the hardware to the skull, and was followed by a thicker layer of dental acrylic for structural support.

Light Delivery

During behavioral testing an external optical fiber (200 μm diameter, 0.22 NA, Doric Lenses, Québec, Canada) was coupled to the implanted fiber optic with a zirconia sleeve. An optical commutator allowed for unrestricted rotation (Doric Lenses, Québec, Canada) (3). Optical stimulation was provided with a 100 mW 473 nm diode pumped solid state laser (OEM Laser Systems, Inc., Salt Lake City, Utah) and controlled by a Master-8 stimulus generator (A.M.P.I., Jerusalem, Israel). Light pulses were recorded with a Digital Lynx data acquisition system (Neuralynx, Bozeman, Calif.) simultaneously with behavioral and neural data. Pulse trains with 5 ms long light pulses at 20 Hz were used for all experiments. The mPFC cell body stimulation experiments used 3 mW light (24 mW/mm² at the fiber tip). The mPFC-DRN axonal stimulation experiments used 20 mW light (159 mW/mm² at the fiber tip). Greater light power was required during the DRN axonal stimulation experiments because of the lower fluorescence at this site, an indicator of lower opsin expression.

Forced Swim Test

We utilized the Porsolt Forced Swim Test for these experiments4. The swim tank was filled with 25° C. water to a height of 40 cm. The induction coil was placed around the tank, and a small magnet was attached to the rat's back foot, as described above. The rats were placed in the FST for 15 minutes on the first day during the light part of the light/dark cycle for pre-exposure. They were then dried with a towel and placed under a heat lamp for 10 minutes to warm before returning to the home cage. Data was collected during a second 15-20 minute test performed 24 hours later. An external fiber optic was suspended above the FST tank and attached to the implanted fiber optic with a zirconia sheath. During experiments with light stimulation, stimulation alternated between on and off in two minute epochs, starting with no stimulation, using the parameters described above for a total of 20 minutes. Induction coil data, video data, and laser pulse time data were collected for all experiments. The FST tank water was changed between each animal.

Open Field Test

An external fiber optic was suspended above the open field and attached to the implanted fiber optic with a zirconia sheath. Rats were placed in the center of a white, dimly lit open field chamber (105×105 cm) and allowed to freely explore the environment. Light stimulation alternated between on and off in three minute epochs, starting with no stimulation, for a total of 15 minutes. A video camera was placed directly above the open field, and locomotor activity was detected and analyzed with Viewer2 software (BiObserve, Fort Lee, N.J.). Laser pulse time data was collected and synchronized to behavioral data.

Anesthetized In Vivo Recordings

Simultaneous dual site recording and optical stimulation of the mPFC and the DRN was performed as described previously3 in anesthetized rats transduced in the mPFC with the AAV5 CaMKIIα-ChR2-EYFP construct. Rats were deeply anesthetized with isoflurane before the start of recording. A midline incision was made and the skin reflected. 2-3 mm diameter craniotomies were made above the mPFC (vertical penetration) and the DRN (30° penetration). A 1 Mohm epoxy-coated tungsten electrode (A-M Systems, Sequim, Wash.) coupled to a 200 μm 0.37 NA optical fiber (Thorlabs Inc., Newton, N.J.) was stereotaxically lowered until a unit was isolated starting at AP 2.7, ML 0.5, DV 3.6 for the mPFC recordings, and AP −7.8, ML 0.5, DV 6.0 (30° penetration) for the DRN recordings. Recorded signals were bandpass filtered between 0.3 and 10 kHz, amplified 10000× (A-M Systems), digitized at 30 kHz (Molecular Devices, Sunnyvale, Calif.) and recorded with Clampex software (Molecular Devices). Optical stimulation was provided with a 100 mW 473 nm diode pumped solid state laser (OEM Laser Systems, Inc., Salt Lake City, Utah). Clampex software was used for both recording neural data and controlling laser output. Light powers between 1 mW (8 mW/mm² at the fiber tip) and 20 mW (159 mW/mm²) were used. At the end of all experiments current was passed through the electrode (50 μA for 30 seconds) to make an electrolytic lesion for anatomical localization.

Histology, Immunohistochemistry, and Confocal Imaging

Rats were deeply anesthetized with Beuthanasia-D and transcardially perfused with ice-cold 4% paraformaldehyde (PFA) in PBS (pH 7.4). Brains were fixed in PFA overnight and then transferred to 30% sucrose in PBS to equilibrate for at least 3 days. 40 μm coronal sections through the mPFC and the DRN were cut on a freezing microtome and stored in cryoprotectant at 4° C. Sections were washed with PBS and incubated for 30 minutes in 0.3% Triton X-100 and 3% normal donkey serum (NDS) in PBS. Sections were incubated with primary antibody overnight in 3% NDS in PBS at 4° C. (rabbit anti-5HT 1:1000, ImmunoStar, Hudson, Wis.). They were then washed in PBS and incubated with secondary antibody conjugated to Cy5 for three hours at room temperature (1:500, Jackson Laboratories, West Grove, Pa.). Sections were washed in PBS and incubated in DAPI (1:50000) for 10 minutes, then washed again and mounted on slides with PVA-DABCO. Images were acquired using a Leica TCS SP5 scanning laser microscope with a 10× air objective or a 40× oil immersion objective.

Data Analysis

Spikes were imported into Offline Sorter software (Plexon Inc, Dallas, Tex.) and sorted offline using waveform features (peak and valley heights) and principal components. Analyses of neural data and behavioral data were done using custom written Matlab (Mathworks, Natick, Mass.) scripts and the Neuroexplorer data analysis package (Nex Technologies, Littleton, Mass.). Statistical significance was defined as p<=0.05 for all analyses. Induction coil data was referenced and zero-phase filtered offline at 1-6 Hz, which preserved the shape of individual kicks while reducing high frequency line noise. The referenced and filtered coil data was then integrated and thresholded (at 10% of the maximum deviation) and the peaks corresponding to individual kicks were detected (FIG. 1). Kicks made during struggling corresponded to large deflections in the recorded signal and could easily be separated from periods of passive floating. Instantaneous kick frequency was defined as the average number of kicks per second in 10 second bins. Automatically scored immobility was determined by scoring epochs with a gap between kicks greater than one second as immobile. The same analysis was performed on induction coil data collected during familiar cage recordings. In this case, the induction coil data was filtered at 1-20 Hz, thresholded at 4% of the maximum deviation, and was not integrated because the unipolar step waveform was more easily detected using these parameters. Automatically scored behavioral data was regressed against manually scored behavioral data to determine the correspondence between scoring methods. R-square and F statistics and p values were derived from this regression analysis. Determination of statistically significant differences in neural firing rate between different behavioral epochs was done using the Wilcoxon rank sum test. Neurons were first tested for differences in firing rate between the pre-FST epoch and the FST epoch. For this analysis neural and behavioral data was binned in 10-second intervals. Neurons were then tested for differences in firing rate between mobile and immobile states during the FST. For this analysis, mobile and immobile behavioral epochs were divided into two different continuous data streams and then statistically tested as above using 10-second bins.

The selectivity index used in FIG. 2 was defined as the difference in firing rate between conditions divided by the sum. Criteria for identifying putative fast spiking inhibitory neurons were a firing rate over 20 Hz and a narrow waveform5. Statistical significance of the behavioral data in FIG. 3 was determined using the Wilcoxon signed rank test. Data was first linearly detrended. The instantaneous average firing rate depicted in FIG. 4 was calculated in 10-second bins, and statistical significance for individual neurons was again calculated using the Wilcoxon rank sum test. The distribution of mobility-immobility differences was tested for changes in variance between stimulated and non-stimulated conditions using the F test for equal variances. Differences in slope were tested with analysis of covariance (ANCOVA).

### Results

Acting to expend energy with vigorous effort under challenging conditions represents a consequential decision for an organism, especially since such action may not always represent the most adaptive behavior. When a vigorous action pattern is selected despite extremely difficult circumstances (rather than a more energy-conserving passive or depressive-type pattern), an assessment may have occurred that anticipated outcomes justify expenditure of energy. Conversely, when an organism selects inactive behavioral patterns in challenging situations, the decision may represent anticipation that effort is likely to be fruitless. Moreover, such anticipation leading to inaction can become maladaptive in human beings, involving clinical symptoms such as psychomotor retardation and hopelessness (core defining features of major depression, a disease with lifetime prevalence of nearly 20% and extensive socioeconomic ramifications (9).

We sought to probe these high-level processes governing behavioral state selection with targeted control of restricted sets of circuit elements in freely-moving mammals. Little is known about the neural underpinnings of such decision-making, nor do broadly-effective medical therapies exist to target these behaviors specifically. However, mounting evidence suggests that the prefrontal cortex (PFC) could be involved; the PFC is responsible for coordinating thought and action, and has been shown to be critical for goal-oriented behavior, planning, and cognitive control (10,11)—all of which are impaired in pathological states such as depression (12-17), consistent with disease-related hypofrontality (5,18).

Moreover, deep brain stimulation (DBS) of the subcallosal cingulate, thought to be analogous to the infralimbic region of medial PFC (mPFC) in rodents, elicits antidepressant effects in treatment-resistant patients (19). Electrical stimulation of the rodent mPFC induces an antidepressant-like reduction in immobility in the forced swim test (20,21), optogenetic stimulation of the mPFC has an antidepressant-like effect on sucrose consumption and social defeat (if both excitatory and inhibitory neurons are concurrently stimulated) (22), and mPFC in rodents appears to mediate resilience and the protective effect of behavioral agency on the acquisition of learned helplessness (23,24) (thought to model key features of major depression) (25). Finally, neuroimaging studies in human patients have been instrumental in focusing attention on brain regions including PFC that exhibit abnormal activity in depression and melancholic states (5,6,26).

Despite these pioneering efforts pointing to the PFC (a broad and complex region with many roles and diverse afferents and efferents), it is unclear which specific neural pathways are involved in real-time selection of goal-oriented behavioral responses to challenging situations. The forced swim test (FST) is relevant to this issue, as a widely-employed behavioral test in rodents (27). In the FST, rodents are placed in an inescapable tank of water and epochs of passive floating or immobility, which are thought to reflect states of behavioral despair (27), are interspersed with epochs of active escape behavior; immobility in the FST is influenced by antidepressant drugs (28) and stress (29). Transitions between active escape and behavioral despair states in the FST are clearly demarcated, in principle providing an unambiguous, instantaneous classification of behavioral state and an opportunity to investigate the neural dynamics underlying the decision to adopt an active behavioral response to challenge. However, to our knowledge, neural activity has never been recorded in behaving animals during the FST because of the fundamental technical obstacles of recording and controlling neural activity in a freely swimming animal.

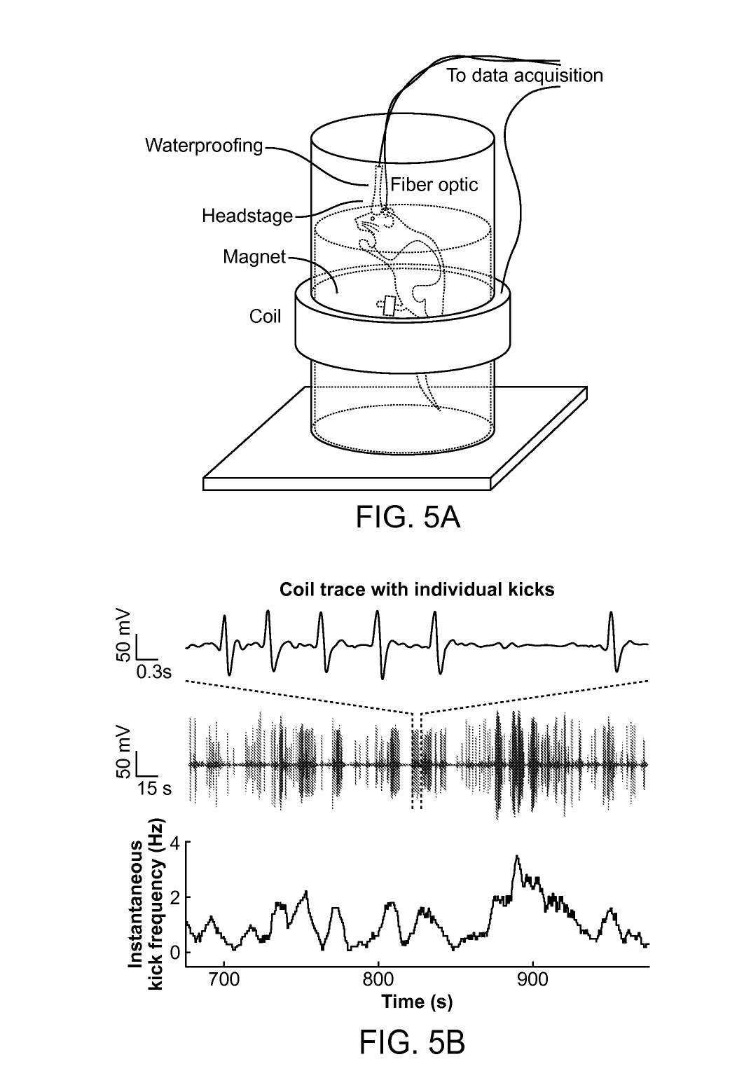

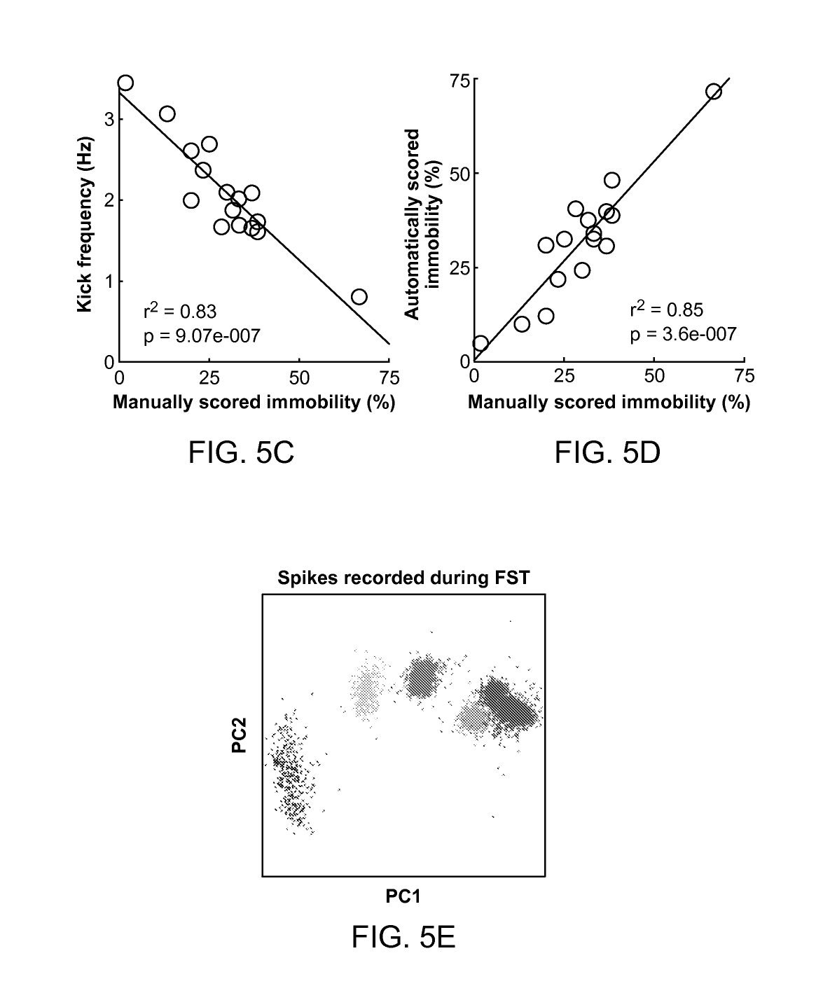

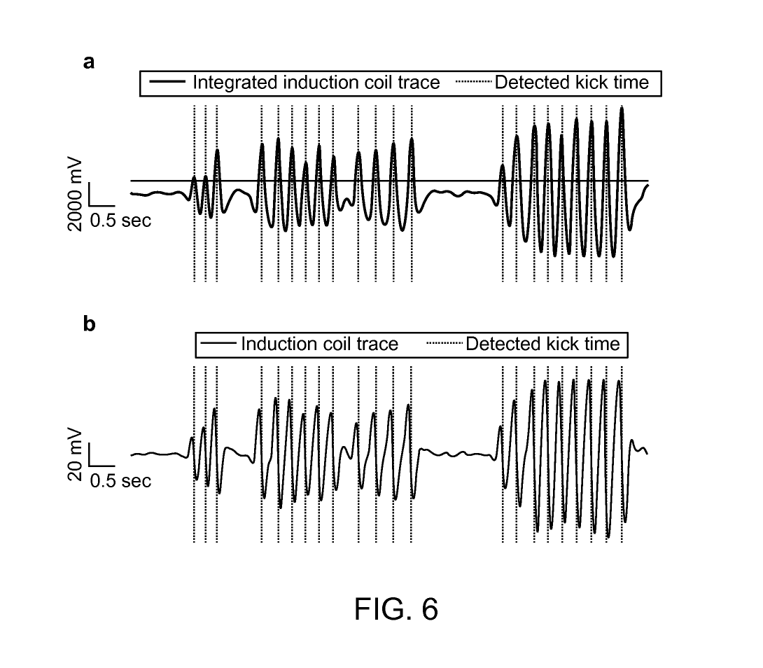

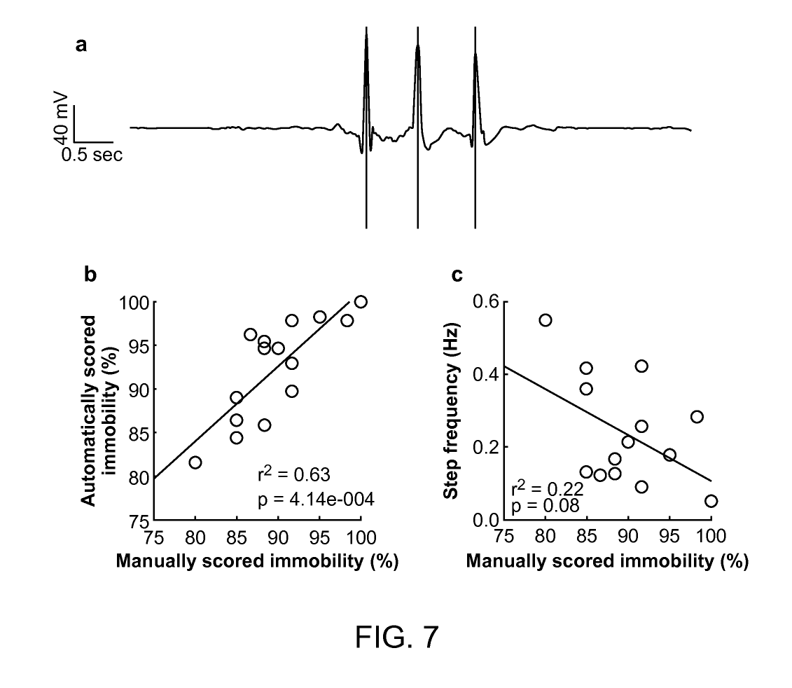

To address this challenge, we developed a new set of methods for recording millisecond-precision neural and behavioral data alongside optogenetic control during the FST (FIG. 5). We designed a magnetic induction method to detect individual swim kicks, in which the FST tank of water was surrounded by an induction coil and a small magnet was attached to the hind paw (FIG. 5a). During the FST each kick induced a current in the coil (FIG. 5b); it was possible to cleanly isolate single kicks (FIG. 6), and kick frequency corresponded well to manually scored immobility (FIG. 5c), providing a reliable measure of behavioral state (FIG. 5d). We additionally employed this method to record mobile and immobile states during activity in a familiar cage, which corresponded well with manually scored data (FIG. 7) as previously observed for tremor measurement in parkinsonian rats (30). In order to record well-isolated single units and local field potentials during swimming, tetrode microdrives or fixed wire arrays were implanted and then waterproofed.

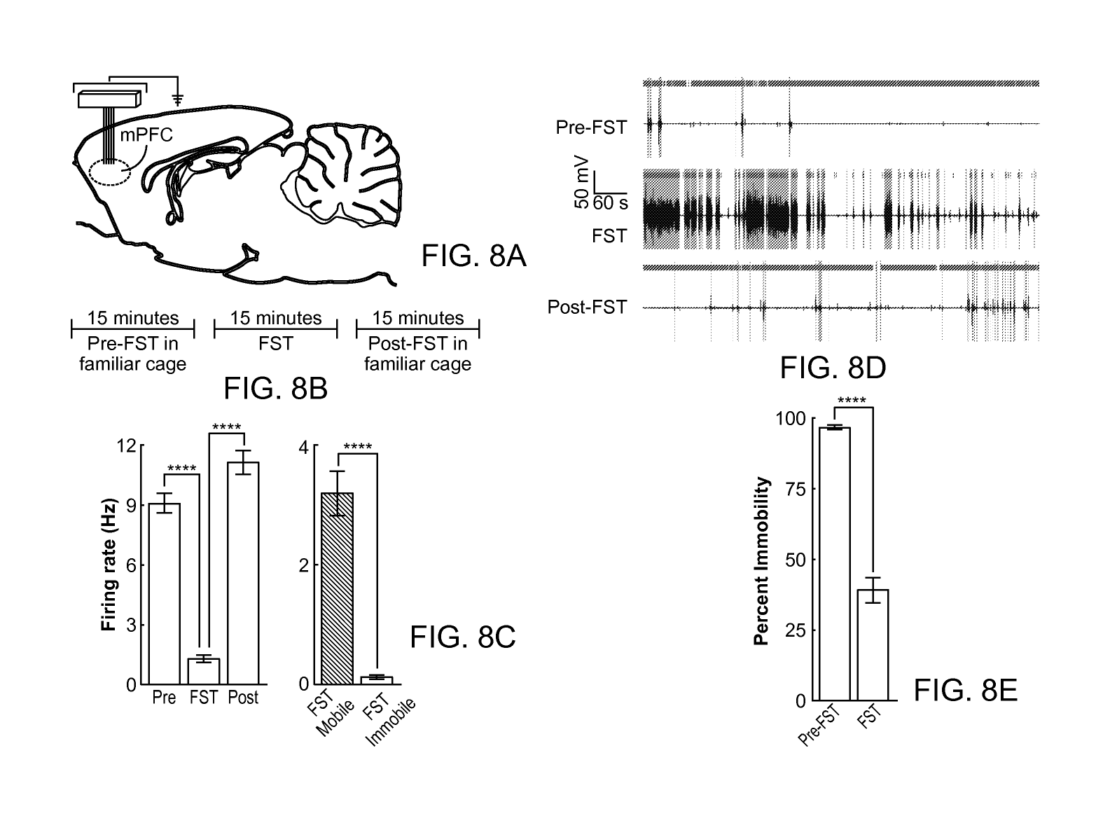

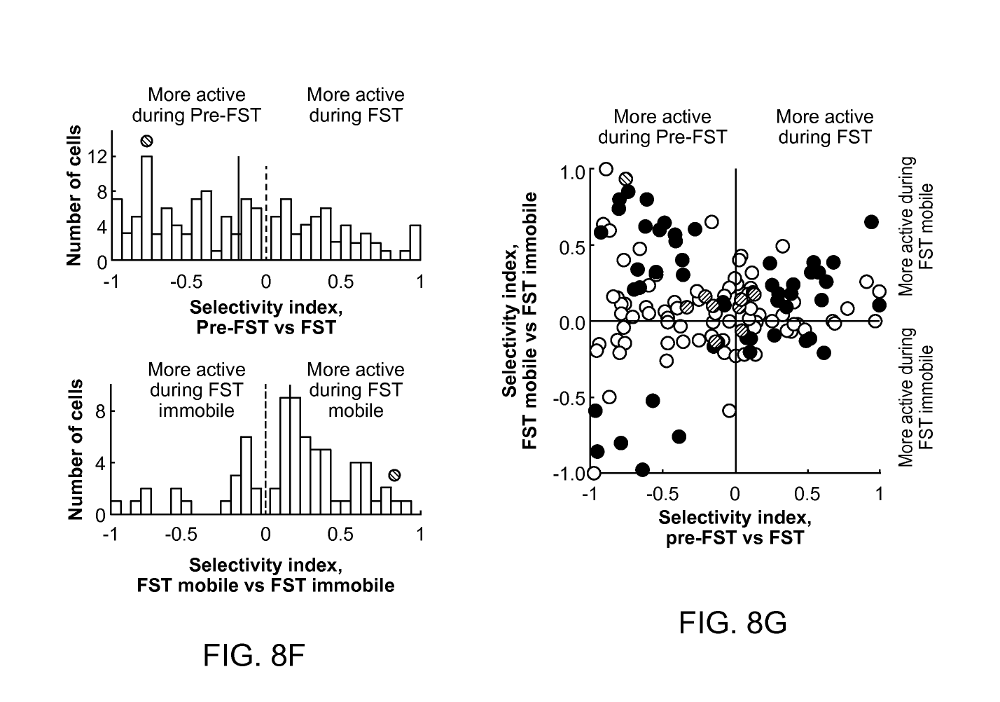

Under these conditions we were reliably able to isolate single units during both immobile and mobile states of the FST (FIG. 5e); indeed, we were able to detect transitions between active escape behavior and immobile states with high temporal precision and to correlate these behaviors with ongoing neural activity (FIG. 8). We recorded neural activity using either a 4-tetrode microdrive (6 rats) or a 24-electrode fixedwire array (5 rats) targeted to the mPFC (FIG. 8a). Three epochs of data were routinely recorded (FIG. 8b): a 15 minute pre-FST epoch in a familiar cage, 15 minutes during the FST, and 15 minutes post-FST after returning to the familiar cage. We found that many mPFC neurons were strongly modulated during behavior in a way that appeared to specifically reflect the decision to act or refrain from action during the FST. An example neuron is shown (FIG. 8c-d). This neuron was highly active during the mostly-immobile pre- and post-FST epochs, but during the FST it stayed active during mobile states and was inhibited during immobile states. This neuron therefore did not simply encode locomotor activity, but was instead specifically inhibited during FST immobility corresponding to traditionally defined states of behavioral despair. We found many neurons in the recorded population ( 23/160, 14%) exhibiting this surprising profile of activity. All rats exhibited minimal motor activity during the pre-FST epoch (greater than 88% immobility for all rats, average 97% immobility) and a moderate to high level of motor activity during the FST epoch (less than 79% immobility for all rats, average 39% immobility, FIG. 8e). Most recorded neurons ( 129/160, 81%) showed a significant change in firing rate between pre-FST and FST epochs (FIG. 8f, top). On average, this population of neurons was inhibited during the FST epoch ( 80/129, 62%), but neurons reflecting the entire range of epoch selectivity were recorded. Many neurons ( 70/160, 44%) also showed a difference in firing rate between mobile and immobile states within the FST epoch (FIG. 8f, bottom). Most of these neurons were activated during mobile states and inhibited during immobile states ( 51/70, 73%), but neurons inhibited during mobile states were also detected.

We then examined the joint distribution of epoch- and mobility-dependent neural selectivity among four quadrants (FIG. 8g), and found it to be highly asymmetric. Two of these, the upper right and lower left quadrants, exhibited a straightforward correspondence between motor activity and neural activity; for example, neurons in the upper right quadrant were more active during the largely mobile FST epoch than during the immobile pre-FST epoch, and, within the FST epoch, were more active during mobile states. The other two quadrants (the upper left and lower right quadrants) showed an inverted correspondence. In the upper left quadrant, neurons that were quieted during the more-active FST epoch were actually activated during escape behaviors within FST, and the neurons in the lower right quadrant did the opposite. The profile of activity found within these groups was therefore not simply dependent on motor activity. Interestingly, there appeared to be many more neurons inhibited during immobile, behavioral despair-like states than there were neurons activated during these states, both in terms of the raw number and in terms of the strength of selectivity exhibited by individual neurons. Finally we noted that putative fast-spiking interneurons exhibited a reduced degree of modulation along both selectivity dimensions, indicating that signals reflecting behavioral response to challenge may be more strongly represented in excitatory or projection neurons.

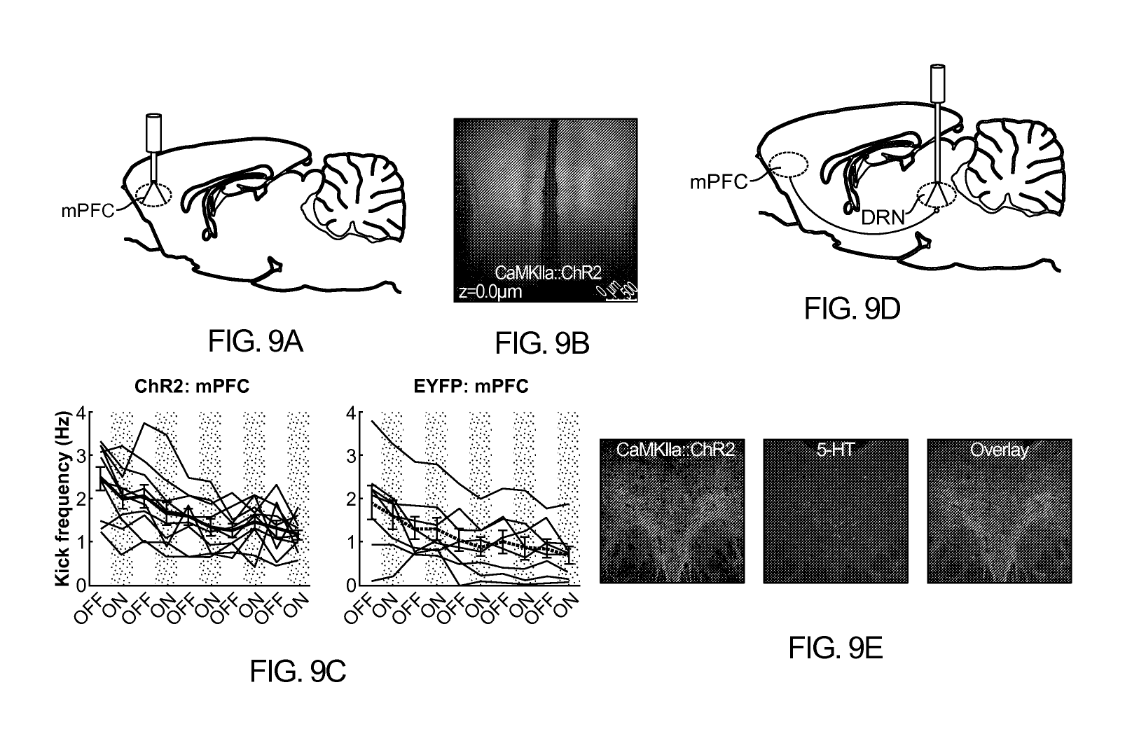

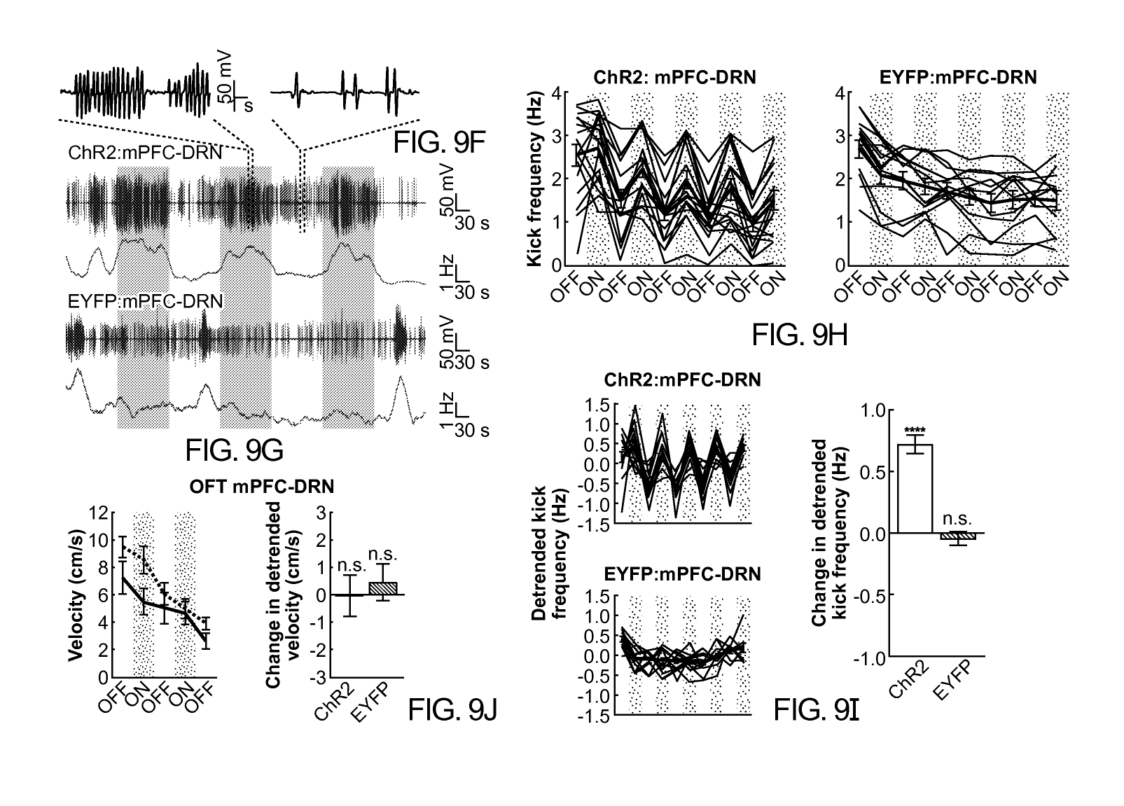

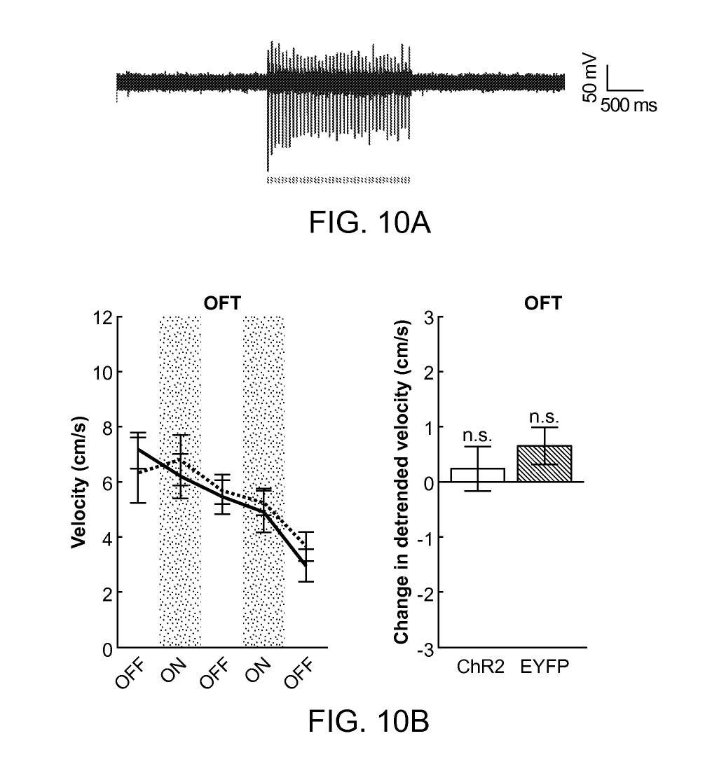

Because the mPFC neurons that we recorded exhibited a range of selectivity profiles, it was not obvious that optogenetically activating local neurons in the mPFC would necessarily have a net effect on behavior during the FST. To test this, we restricted opsin expression to excitatory neurons within the mPFC using an adeno-associated viral vector (AAV5) expressing channelrhodopsin-2 fused to enhanced yellow fluorescent protein (ChR2-EYFP) under the control of the CaMKIIα promoter. Virus was infused into the mPFC and a miniature fiber optic was implanted over the prelimbic region (FIG. 9a-b). Functional targeting of these neurons was confirmed by anesthetized optrode recordings in the mPFC and demonstration of spiking activity upon illumination (FIG. 10a), but surprisingly, when these neurons were illuminated in two-minute epochs during the FST, we found that stimulation was not sufficient to cause even a slight reduction in immobility (FIG. 9c). We also tested these rats on the open field test (OFT) to assess stimulation-induced changes in locomotor activity and similarly did not find evidence of a gross locomotor effect (FIG. 10b). One interpretation of these FST results is that local PFC neurons may correlate with, but are not causally involved in, the behavioral state changes associated with mobility and immobility; alternatively, it could be that some local PFC neurons are so involved, but others are not or are opposed in causal function, and when driven together no net effect on behavior is seen.

We therefore next hypothesized that it could be possible to induce a change in motivated behavioral state by restricting optogenetic stimulation to a reduced population of mPFC neurons. The mPFC is known to project to several downstream brain regions that have been implicated in motivated behavior and depression (31); among these is the dorsal raphe nucleus (DRN) (32), largest of the nine serotonergic nuclei (33,34) and implicated in major depressive disorder8. The mPFC exerts control over both neural activity in the DRN and extracellular 5-HT levels (23,35), and antidepressant-like effects of mPFC electrical stimulation appear to depend on an intact 5-HT system (20), but the projection from the mPFC to the DRN has never been directly shown to have an effect on behavior.

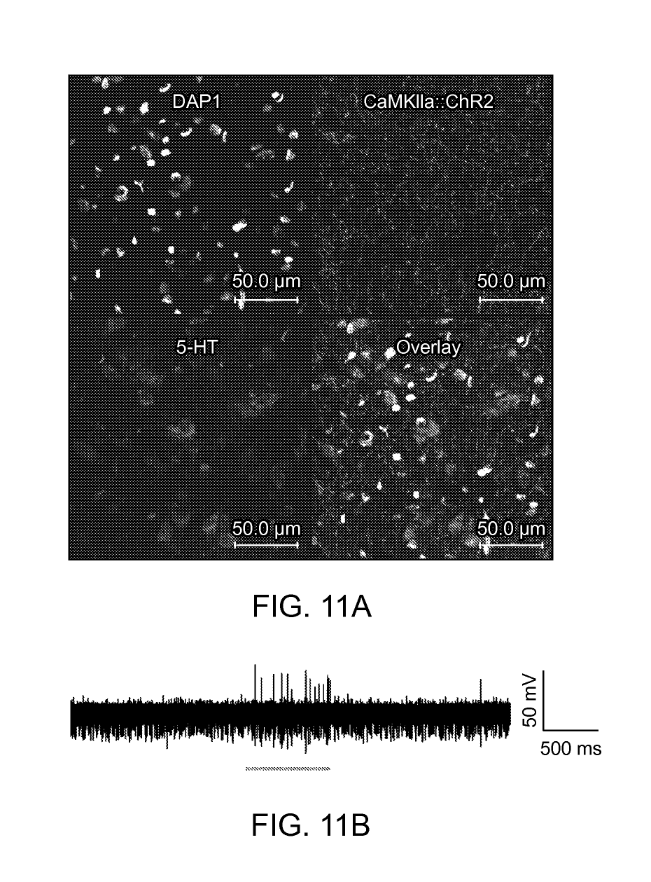

In order to specifically activate the mPFC-DRN projection, we first transduced excitatory neurons in the mPFC with ChR2-EYFP under the control of the CaMKIIα promoter using an AAV5 viral vector (FIG. 9d), which led to robust ChR2-EYFP expression in mPFC axons in the DRN (FIG. 9e; FIG. 11a). We restricted activation to the subpopulation of excitatory neurons in the mPFC that project to the DRN by implanting a miniature fiber optic over the DRN and selectively illuminating the mPFC axons in this region (FIG. 9d). Functional targeting was confirmed by anesthetized optrode recordings in the DRN and demonstration of spiking activity of DRN neurons upon illumination of mPFC axons (FIG. 10b).

When the axons of ChR2-EYFP-expressing mPFC neurons in the DRN were stimulated during the FST, a profound behavioral effect resulted. Example induction-coil behavioral traces from two rats are shown (one ChR2-EYFP and one EYFP rat, FIG. 9f-g), demonstrating a robust increase in kick frequency during each light epoch in the ChR2-EYFP case but not in the control EYFP case. This behavioral effect was present in most rats and was rapid, reversible, and repeatable (FIG. 9h-i). Importantly, stimulation of this projection did not affect locomotor activity in the open field (FIG. 9j), demonstrating again that the increase in escape behaviors seen during the FST was not the result of nonspecific motor activation. This result stood in marked contrast to the lack of effect seen with driving all mPFC excitatory neurons nonspecifically as shown in FIG. 9c, demonstrating the importance of resolving subpopulations defined by projection target, and illustrating a causal role of a specific PFC-to-brainstem neural pathway in driving goal-oriented behavioral responses to a challenging environment.

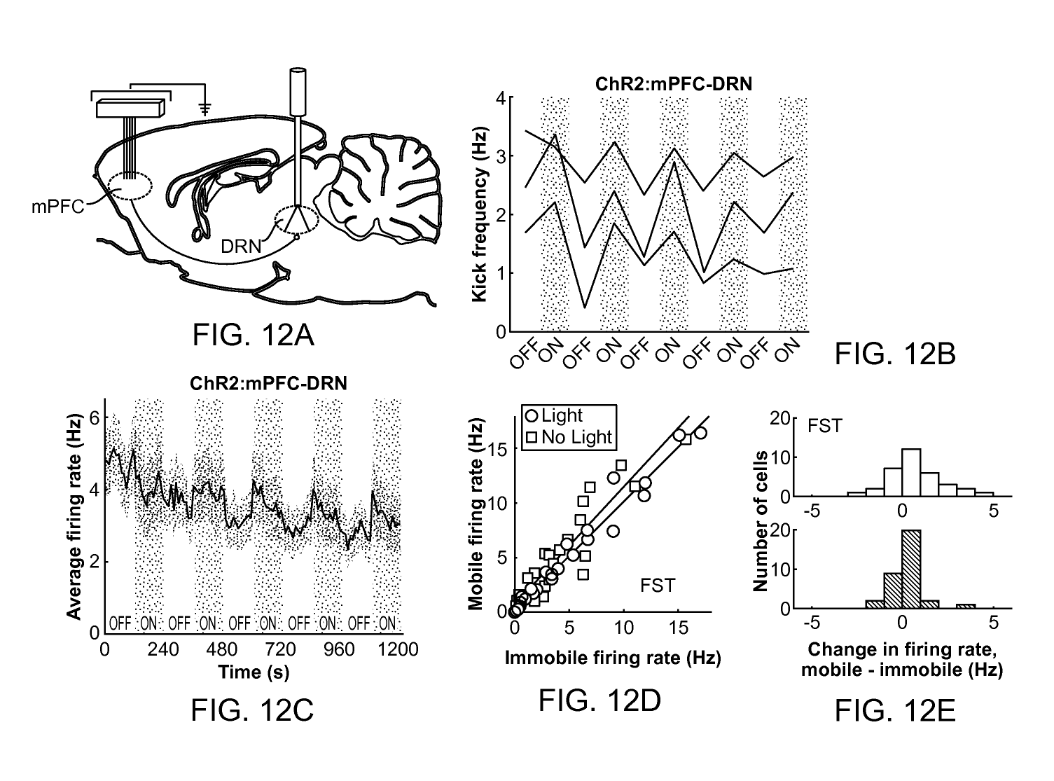

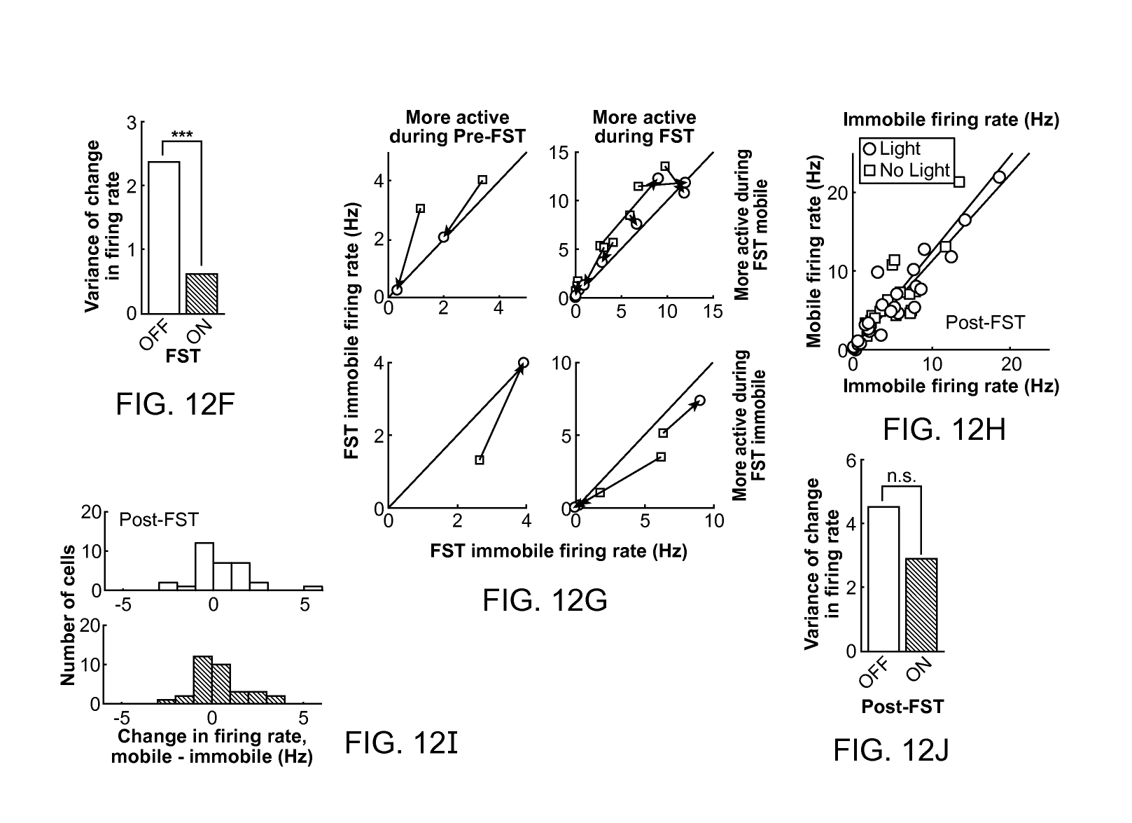

Finally, we explored how this optogenetically-induced behavioral effect might influence mPFC neural coding of behavioral state during the FST. For this experiment, we expressed ChR2-EYFP in mPFC principal neurons and implanted a fiber optic over the DRN in order to specifically activate cells with this projection. In addition, we implanted a 24-electrode fixed wire array over the mPFC to record neural activity in these neurons while simultaneously stimulating the mPFC-DRN projection (FIG. 12a). We recorded neural activity from 3 rats, all of which showed a robust light-induced behavioral effect (FIG. 12b). Moreover, light stimulation influenced firing rate in almost all recorded mPFC neurons ( 31/34, 91%). The average firing rate across the population increased slightly during stimulation epochs (FIG. 12c), although more neurons were significantly inhibited ( 22/31, 71%) than excited ( 9/31, 29%) by stimulation, consistent with selection of a specific behavioral state and suggesting that the net effect of mPFC->DRN stimulation on mPFC is a widespread weak inhibition coupled with a relatively strong sparse excitation of the mPFC network. We also noted that information related to behavioral state was reduced in the PFC during stimulation epochs.

When the difference in firing rate between mobile and immobile states was tested during epochs without light stimulation in the FST, 62% (21/34) of neurons were significantly modulated. However, when these same neurons were tested for differences in firing rate between mobile and immobile states during light stimulation, the proportion that was significantly selective fell dramatically to 21% ( 7/34). Examination of mobile vs. immobile firing rates for each neuron illustrates this effect (FIG. 12d); firing rates during stimulation showed less dependence on mobility state than firing rates during epochs without stimulation; these points have a tighter distribution around the best-fit line. We examined the raw differences in firing rates between mobile and immobile states and observed that the distribution of this difference was significantly narrower during stimulation (FIG. 12e-f). There was no significant difference in slope between the stimulated and non-stimulated conditions (ANCOVA, p=0.2041).

Dividing the population of recorded neurons into the four quadrants described in FIG. 8g, we noted that the reduction in mobility-immobility encoding held true for all cell types (FIG. 12g), and that this reduction is seen whether neurons increase, decrease, or maintain their average firing rate. This reduction in mPFC encoding of behavioral state during light stimulation may reflect a decreased need for further recruiting this specific endogenous PFC activity pattern in the presence of exogenously applied goal orientation-driving stimulation. Intriguingly, light stimulation did not have an effect on mobility encoding during the post-FST epoch (FIG. 12h-j).

Here, we have probed both neural correlates and causal neural pathways involved in the selection of goal-oriented behavior in a challenging situation, using novel technology permitting electrical recordings and optogenetic control in the forced swim test in combination with high speed readout of instantaneous behavioral state. We have demonstrated the existence of different physiologically-defined mPFC neural populations—one selectively inhibited during epochs of behavioral despair-like states, and the other selectively activated. We have also demonstrated that, while activation of all excitatory neurons in the mPFC does not have a net effect on this behavior, selective activation of those mPFC neurons that project to the DRN has a profound, rapid, and reversible effect on the selection of the active behavioral state in response to challenge without nonspecific motor activation.

In summary, these physiological and behavioral results describe the neural dynamics underlying the behavioral response to challenging situations and demonstrate the causal importance of mPFC control of the DRN in implementing this response, with potential implications for understanding both normal and pathological states of behavior pattern selection.

FIGS. 5A-E: The Automated FST Provides a High Temporal Resolution Behavioral Readout that can be Synchronized with Simultaneously Recorded Neural Data.

a) A schematic of the automated FST. The tank of water is surrounded by a coil of wire and a magnet is comfortably attached to the rat's back paw. Movement of the magnet within the coil during swimming induces a current that can be recorded. In order to permit concurrent neural recordings the headstage is waterproofed. An optical fiber can be included for simultaneous optical stimulation. b) Example FST coil traces. Coil voltage is depicted. Top: a short, 6 second behavioral coil trace depicting individual kicks. Middle: a longer, 5 minute behavioral coil trace depicting activity on a longer timescale. Bottom: Instantaneous kick frequency estimated from the 5-minute coil trace. c) Average kick frequency corresponds well to manually scored immobility estimates. d) Estimates of FST immobility derived from the induction coil correspond tightly to manually scored immobility estimates. e) 4 well-isolated single mPFC units recorded during the FST.

FIGS. 6A and 6B: Detection of Individual Kicks in the Forced Swim Test.

a) The induction coil trace is first filtered (1-6 Hz) and then integrated to yield a peak at the midpoint of each kick. The integrated trace is then thresholded (10% of the maximum deviation) and the peaks are detected. The threshold is shown in gray. b) The filtered coil trace before integration. Kick times correspond to the midpoint of each kick.

FIGS. 7A-C: The Magnetic Induction Method can be Used to Detect Immobility in a Cage.

a) The induction coil trace is filtered (1-20 Hz), thresholded (4% of the maximum deviation), and the peaks are detected. The cage coil trace is not integrated before peak detection because of the unipolar waveform associated with steps. b) Automatically scored cage immobility corresponds well to manually scored cage immobility. c) Average step frequency corresponds well to manually scored immobility.

FIGS. 8A-G: Prefrontal Neuronal Activity Encodes FST Behavioral State.

a) A 4-tetrode microdrive (6 rats) or a 24-electrode fixed wire array (5 rats) was implanted over the mPFC. b) We recorded 15 minutes of pre-FST data in a familiar cage (pre-FST), 15 minutes during the FST (FST), and 15 minutes following the FST (post FST) in the familiar cage. c) Bar plot of an example neuron that is specifically inhibited during immobile states in the FST. This neuron fires at a high rate during largely-immobile Preand Post-FST epochs and mobile FST states, but is specifically inhibited during immobile FST states (Wilcoxon rank sum test, * p<0.05; ** p<0.01; ***p<0.001; ****p<0.0001). d) Raster plot of the same neuron. Coil trace in black, mobile states in purple, spikes in red. Top: pre-FST activity in the familiar cage. This neuron fires at a high rate throughout the pre-FST epoch, in which the rat is almost entirely immobile (98% immobility). Middle: activity during the FST. This neuron fires at a high rate during mobile states, but is inhibited during immobile states corresponding to behavioral despair-like states. Bottom: post-FST activity in the familiar cage. This neuron again fires robustly throughout the post-FST epoch, in which the rat is mostly immobile (94% immobility). e) Behavioral data during the pre-FST and FST test epochs from all 11 rats. Rats were almost entirely immobile during the pre-FST period in the familiar cage (97% immobile), but were much more active during the FST (39% immobile). f) Distribution of population selectivity indices (see Supplementary Methods). Top: pre-FST vs. FST epochs. All neurons significantly selective for pre-FST vs. FST are shown. A wide range of selectivity profiles is represented. Bottom: mobile vs. immobile FST states. All neurons significantly selective for mobile vs. immobile FST state are shown. Most neurons fired more during mobile FST states than during immobile FST states. g) Joint distribution of selectivity indices. The upper left quadrant corresponds to neurons that were specifically inhibited during immobile states in the FST, while the lower right quadrant depicts neurons that were specifically activated during these states. Black circles: neurons selective for both task epoch and mobility. Red circle: example neuron. Blue circles: putative inhibitory fast-spiking neurons. Gray circles: non-significantly selective neurons. All recorded neurons are shown.

FIGS. 9A-J: Optogenetic Stimulation of mPFC Axons in the DRN, but not Excitatory mPFC Cell Bodies, Induces Rapid and Reversible Behavioral Activation in a Challenging Situation.

a) AAV5 CaMKIIα-ChR2-EYFP or CaMKIIα-EYFP was infused bilaterally in the mPFC and a fiber optic was implanted over the infected cell bodies. b) EYFP fluorescence in the mPFC. c) Behavioral data from ChR2-EYFP (left, n=10) and EYFP (right, n=8) rats. Illumination of excitatory mPFC cell bodies in ChR2-EYFP rats did not induce a behavioral effect (detrended data, light on vs light off, Wilcoxon signed rank test, p=0.23). Gray lines represent individual rats, while thicker lines depict behavioral averages for ChR2-EYFP (red) or EYFP (black) rats. Blue bars indicate light on. d) AAV5 CaMKIIα-ChR2-EYFP or CaMKIIα-EYFP was infused bilaterally in the mPFC and a fiber optic was implanted over the DRN in order to specifically activate mPFC-DRN axons. e) EYFP fluorescence in mPFC axons in the DRN (immunostained for 5-HT). f) Behavioral data from one ChR2-EYFP-expressing rat. Top, middle: coil trace. Bottom: kick frequency. Kick frequency is increased during light stimulation. Blue bars indicate light on. g) Behavioral data from one EYFP-expressing rat. Top: coil trace. Bottom: kick frequency. Kick frequency is not affected by light stimulation. h) Behavioral data from all rats. Left: ChR2-EYFP rats (n=16). Illumination of ChR2-expressing mPFC axons in the DRN induced a rapid and reversible behavioral activation in the FST. Right: EYFP rats (n=12). Illumination of EYFP-expressing mPFC axons in the DRN did not affect behavior. i) Linearly detrended data from h. Kick frequency during light stimulation was significantly increased during illumination in ChR2-EYFP rats (Wilcoxon signed rank test, p=1.04e-11) but not EYFP rats (Wilcoxon signed rank test, p=0.39; * p<0.05; ** p<0.01; ***p<0.001; **** p<0.0001). j) Open field test. Light stimulation of DRN-projecting mPFC neurons did not have a nonspecific effect on locomotor activity in either ChR2-EYFP rats (n=12, Wilcoxon signed rank test, p=0.59) or EYFP rats (n=12, Wilcoxon signed rank test, p=0.71).

FIGS. 10A and 10B: Optogenetic Stimulation of the Rat mPFC.

a) AAV5 CaMKIIα-ChR2-EYFP was infused bilaterally in the mPFC. An optrode recording detected spiking activity in the mPFC induced by local cell body illumination. b) Open field test. AAV5 CaMKIIα-ChR2-EYFP or AAV5 CaMKIIα-EYFP was infused bilaterally in the mPFC. Light stimulation of the mPFC did not affect velocity in either ChR2-EYFP rats (Wilcoxon signed rank test, p=0.50, n=10) or EYFP rats (Wilcoxon signed rank test, p=0.09, n=8). Red line indicates the ChR2-EYFP group average. Gray line indicates the EYFP group average. Blue bars indicate light on. Significance calculations were performed on detrended data.

FIGS. 11A and 11B: DRN Histology and Optrode Recording.

a) AAV5 CaMKIIα-ChR2-EYFP was infused bilaterally in the mPFC. EYFP fluorescence in mPFC axons in the DRN is shown in green, immunostaining for 5-HT is shown in red, and DAPI staining for nuclei is shown in white. b) AAV5 CaMKIIα-ChR2-EYFP was infused bilaterally in the mPFC. An optrode recording in the DRN detected local spiking activity induced by illumination of mPFC axons in the DRN. Spikes were not elicited with every light pulse. 12 overlaid traces are shown.

FIGS. 12A-J: Optogenetic Stimulation of DRN-Projecting mPFC Neurons Decreases mPFC Encoding of Mobility.

a) AAV5 CaMKIIα-ChR2-EYFP was infused bilaterally into the mPFC, and a fiber optic was implanted over the DRN. A 24-electrode fixed-wire array was targeted to the mPFC. b) All three injected and implanted rats showed a robust increase in FST mobility during stimulation. c) Light stimulation of the mPFC-DRN induced a modest increase in average mPFC firing rate (but see text). d) Illumination of mPFC axons in the DRN decreased mPFC encoding of mobility state during the FST. Each point represents one neuron. Black squares depict mobile vs. immobile firing rate of neurons without light stimulation, while blue circles depict mobile vs. immobile firing rates with light stimulation. Light stimulation causes these points to cluster tightly around the best-fit line. There was not a significant change in slope with light stimulation (ANCOVA, p=0.20). e-f) Histograms of the change in firing rate between mobile and immobile states. Top: no light stimulation. Bottom: light stimulation. Illumination decreases the variance in this distribution (F test for equal variance, p=2.11e-4), indicating decreased encoding of mobility state in these neurons. g) All four quadrants of neurons (see FIG. 2f) show a decrease in encoding of mobility state with light stimulation. h-j) Stimulation does not have a significant effect on mobility state encoding when rats are in a familiar cage, and not engaged in the FST.

### REFERENCES

1. McGuire et al. Proceedings of the National Academy of Sciences of the United States of America 107: 7922-6 (2010)
2. Ridderinkhof et al. Brain and cognition 56: 129-40 (2004)
3. Rubia et al. NeuroImage 20: 351-358 (2003)
4. Aron et al. Nature neurosicence 6: 115-6 (2003)
5. Baxter et al. Archives of general psychiatry 46: 243-50 (1989)
6. Mayberg et al. The American journal of psychiatry 156: 675-82 (1999)
7. Starkstein et al. Brain: a journal of neurology 110 (Pt 4): 1045-59 (1987)
8. Maes et al. Psychopharmacology: the fourth generation of progress 933-44 (1995)
9. Kessler et al. World psychiatry: official journal of the World Psychiatric Association (WPA) 6: 168-76 (2007)
10. Miller et al. Annual review of neuroscience 24: 167-202 (2001)
11. Fuster. The Prefrontal Cortex, Fourth Edition (2008)
12. Davidson et al. Annual review of psychology 53: 545-74 (2002)
13. Elliot et al. Psychological medicine 27L 931042 (1997)
14. Austin. The British Journal of Psychiatry 178: 200-206 (2001)
15. Ingram et al. Cognitive Therapy and Research 18: 317-332 (1994)
16. Dalgleish et al. Clinical Psychology Review 10: 589-604 (1990)
17. Fales et al. Biological Psychiatry 63: 377-384 (2008)
18. Bench et al. Psychological medicine 22: 607-15 (1992)
19. Mayberg et al. Neuron 45: 651-60 (2005)
20. Hamani et al. Biological psychiatry 67: 117-24 (2010)
21. Hamani et al. Journal of psychiatric research 44: 683-7 (2010)
22. Covington et al. The Journal of neuroscience: the official journal of the Society for Neuroscience 30: 16082-90 (2010)
23. Amat et al. Nature neuroscience 8: 365-71 (2005)
24. Amat et al. Neuroscience 154: 1178-86 (2008)
25. Abramson et al. Journal of abnormal psychology 87: 49-74 (1978)
26. Drevets. Current opinion in neurobiology 22: 240-9 (2001)
27. Porsolt et al. Nature 266: 730-732 (1977)
28. Cryan et al. Neuroscience and biobehavioral reviews 29: 547-69 (2005)
29. Willner. Neuropsychobiology 52: 90-110 (2005)
30. Dill et al. Journal of applied physiology 24: 598-9 (1968)
31. Vertes. Synapse (New York, N. Y.) 51: 32-58 (2004)
32. Gonsalves et al. Brain research bulletin 78: 240-7 (2009)
33. Lidov et al. Neuroscience 5: 207-27 (1980)
34. Michelsen et al. Brain research reviews 55: 329-42 (2007)
35. Celada et al. The Journal of neuroscience: the official journal of the Society for Neuroscience 21: 9917-29 (2001)
36. Ressler et al. Nature neuroscience 10: 1116-24 (2007)

### Example 3

### Role of Dopamine Neurons in Conditioned Placement Aversion

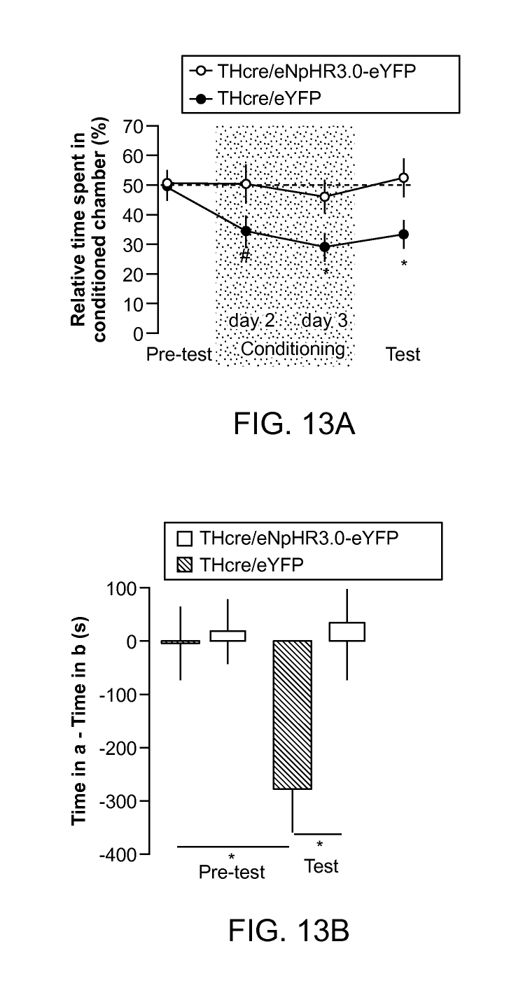

Mice were as described in Example 1. THcre⁺/eNpHR3.0-eYFP mice and THcre⁺/eNpHR3.0-eYFP mice were subjected to a conditioned place test. The results are shown in FIGS. 13A and 13B.

FIG. 13A.

THcre+ mice infected in the VTA with AAV5-flox-eNpHR3.0-eYFP or AAV5-flox-eYFP. The two groups exhibited a significantly different performance (F(1,16)=5, p≦0.001). Two conditioning session (amber box) are sufficient to induce an aversion of the conditioning chamber in THcre+/eNpHR3.0-eYFP mice (**p≦0.01, t(3.3;14)). Only THcre+/eNpHR3.0-eYFP mice showed aversion during the whole experiment (F(3,21)=7.7 ***p≦0.001) (THcre+/eNpHR3.0-eYFP vsTHcre+/eYFP on conditioning day 2 *p≦0.05, t(2.3;16); THcre+/eNpHR3.0-eYFP vsTHcre+/eYFP on test day *p≦0.05, t(2.2;16)). In FIG. 13A, the upper line is Thcre/eYFP; the lower line is THcre⁺/eNpHR2.0-eYFP. FIG. 13B. Note that during the pre-test there is no initial aversion as the time spent by the mice in both chambers is equal. After conditioning, mice expressing eNpHR3.0-eYFP in VTA DA cells show a clear aversion on the test day for the conditioned chamber a (pretest day vs test day for THcre+/eNpHR3.0-eYFP mice *p≦0.05, t(2.5;14) but not control mice (THcre+/eNpHR3.0-eYFP vs THcre+/eYFP on test day *p≦0.05 t2.2;16 n=10). In FIG. 13B, the left-hand bars in the “pre-test” and “test” data sets are THcre/eYFP; the right-hand bars in the “pre-test” and “test” data sets are THcre⁺/eNpHR3.0-eYFP.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A method for identifying a candidate agent treating depression in an individual, comprising:
contacting a rodent that expresses an active optogenetic inhibitor of neuronal activity in ventral tegmental area (VTA) dopaminergic neurons with a test agent, and
determining the effect of the test agent on a behavior of the rodent in a depression assay,
wherein reduction in a depressive behavior of the rodent contacted with the test agent, compared to the behavior of a control rodent that has not been contacted with the test agent, indicates that the test agent is a candidate agent for treating depression.

2. The method of claim 1, wherein the active optogenetic inhibitor is a halorhodopsin (NpHR) polypeptide comprising an amino acid sequence having at least about 95% amino acid sequence identity to the NpHR amino acid sequence set forth in SEQ ID NO:1.

3. The method of claim 2, wherein the NpHR is encoded by a nucleotide sequence that is operably linked to a promoter that provides for expression of the NpHR in a dopaminergic neuron.

4. The method of claim 2, wherein the NpHR comprises an endoplasmic reticulum export signal and a membrane trafficking signal.

5. The method of claim 1, wherein said determining is carried out after, or concurrently with, exposing the VTA to light at a wavelength that activates the optogenetic inhibitor.

6. The method of claim 1, wherein said depression assay is a forced swim test, a tail suspension test, or a conditioned place aversion test.

7. A transgenic rodent comprising dopaminergic neurons of the ventral tegmental area that express an optogenetic inhibitor of neuronal function.

8. The transgenic rodent of claim 7, wherein the optogenetic inhibitor of neuronal function is a halorhodopsin (NpHR) polypeptide comprising an amino acid sequence having at least about 95% amino acid sequence identity to the NpHR amino acid sequence set forth in SEQ ID NO:1.

9. The transgenic rodent of claim 8, wherein the NpHR is encoded by a nucleotide sequence that is operably linked to a promoter that provides for expression of the NpHR in a dopaminergic neuron.

10. The transgenic rodent of claim 8, wherein the NpHR comprises an endoplasmic reticulum export signal and a membrane trafficking signal.

11. A method for identifying a candidate agent for treating depression in an individual, comprising:
contacting a rodent that expresses an active optogenetic activator of neuronal activity in ventral tegmental area (VTA) dopaminergic neurons with a test agent, and
determining the effect of the test agent on a behavior of the rodent in a depression assay,
wherein reduction in a depressive behavior of the rodent contacted with the test agent, compared to the behavior of a control rodent that has not been contacted with the test agent, indicates that the test agent is a candidate agent for treating depression.

12. The method of claim 11, wherein said determining is carried out without exposing the VTA to light at a wavelength that activates the optogenetic inhibitor.

13. The method of claim 11, wherein the optogenetic activator of neuronal activity is a channelrhodopsin polypeptide comprising an amino acid sequence having at least about 95% amino acid sequence identity to the channelrhodopsin amino acid sequence set forth in SEQ ID NO:5.

14. The method of claim 13, wherein the channelrhodopsin is encoded by a nucleotide sequence that is operably linked to a promoter that provides for expression of the channelrhodopsin in a dopaminergic neuron.

15. The method of claim 13, wherein the channelrhodopsin comprises an endoplasmic reticulum export signal and a membrane trafficking signal.

16. A method for screening an agent for the ability to promote depression in an individual, the method comprising:
contacting a rodent that expresses an active optogenetic activator of neuronal activity in ventral tegmental area (VTA) dopaminergic neurons with an agent, and
determining the effect of the agent on the behavior of the rodent in a depression assay,
wherein a depressive behavior of the rodent contacted with the agent, compared to the behavior of a control rodent that has not been contacted with the agent, indicates that the agent promotes depression.

17. The method of claim 16, wherein the active optogenetic activator is a channelrhodopsin.

18. The method of claim 16, wherein the active optogenetic activator of neuronal activity in VTA dopaminergic neurons is activated upon exposure of the VTA to light of an activating wavelength.

19. A method for screening an agent for the ability to promote depression in an individual, the method comprising:
contacting a rodent that expresses an active optogenetic activator of neuronal activity in medial prefrontal cortex (mPFC) excitatory neurons with an agent, and
determining the effect of the agent on the behavior of the rodent in a depression assay,
wherein a depressive behavior of the rodent contacted with the agent, compared to the behavior of a control rodent that has not been contacted with the agent, indicates that the agent promotes depression.

20. The method according to claim 19, wherein the active optogenetic activator is a channelrhodopsin.

21. The method according to claim 19, wherein said contacting is carried out before or concurrently with exposing the dorsal raphe nucleus (DRN) to light of a wavelength that activates the optogenetic activator.

22. A method for identifying a candidate agent for treating an adverse psychological state in an individual, the method comprising:
contacting a rodent that expresses an active optogenetic inhibitor of neuronal activity in ventral tegmental area (VTA) dopaminergic neurons with a test agent, and
determining the effect of the test agent on a behavior of the rodent in a conditioned place aversion (CPA) test,
wherein modulation in the CPA response behavior of the rodent contacted with the test agent, compared to the behavior of a control rodent that has not been contacted with the test agent, indicates that the test agent is a candidate agent for treating an adverse psychological state in an individual.

23. The method of claim 22, wherein the adverse psychological state is dysphoria, anhedonia, depression, suicidality, or anxiety.

24. The method of claim 22, wherein the active optogenetic inhibitor is a halorhodopsin (NpHR) polypeptide comprising an amino acid sequence having at least about 95% amino acid sequence identity to the NpHR amino acid sequence set forth in SEQ ID NO:1.

25. The method of claim 24, wherein the NpHR is encoded by a nucleotide sequence that is operably linked to a promoter that provides for expression of the NpHR in a dopaminergic neuron.

26. The method of claim 24, wherein the NpHR comprises an endoplasmic reticulum export signal and a membrane trafficking signal.

27. The method of claim 22, wherein said determining is carried out after, or concurrently with, exposing the VTA to light at a wavelength that activates the optogenetic inhibitor.


SEQUENCE LISTING (22 sequences)

SEQ ID NO: 1 - protein, 273 aa, Artificial sequence
  Synthetic amino acid sequence
VTQRELFEFVLNDPLLASSLYINIALAGLSILLFVFMTRGLDDPRAKLIAVSTILVPVVSIASYTGLASGLTISVLEMPAGHFAEGSSVMLGGEEVDGVVTMWGRYLTWALSTPMILLALGLLAGSNATKLFTAITFDIAMCVTGLAAALTTSSHLMRWFWYAISCACFLVVLYILLVEWAQDAKAAGTADMFNTLKLLTVVMWLGYPIVWALGVEGIAVLPVGVTSWGYSFLDIVAKYIFAFLLLNYLTSNESVVSGSILDVPSASGTPADD

SEQ ID NO: 2 - protein, 559 aa, Artificial sequence
  Synthetic amino acid sequence
MTETLPPVTESAVALQAEVTQRELFEFVLNDPLLASSLYINIALAGLSILLFVFMTRGLDDPRAKLIAVSTILVPVVSIASYTGLASGLTISVLEMPAGHFAEGSSVMLGGEEVDGVVTMWGRYLTWALSTPMILLALGLLAGSNATKLFTAITFDIAMCVTGLAAALTTSSHLMRWFWYAISCACFLVVLYILLVEWAQDAKAAGTADMFNTLKLLTVVMWLGYPIVWALGVEGIAVLPVGVTSWGYSFLDIVAKYIFAFLLLNYLTSNESVVSGSILDVPSASGTPADDAAAKSRITSEGEYIPLDQIDINVVSKGEELFTGVVPILVELDGDVNGHKFSVSGEGEGDATYGKLTLKFICTTGKLPVPWPTLVTTFGYGLQCFARYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSYQSALSKDPNEKRDHMVLLEFVTAAGITLGMDELYKFCYENEV

SEQ ID NO: 3 - protein, 542 aa, Artificial sequence
  Synthetic amino acid sequence
MVTQRELFEFVLNDPLLASSLYINIALAGLSILLFVFMTRGLDDPRAKLIAVSTILVPVVSIASYTGLASGLTISVLEMPAGHFAEGSSVMLGGEEVDGVVTMWGRYLTWALSTPMILLALGLLAGSNATKLFTAITFDIAMCVTGLAAALTTSSHLMRWFWYAISCACFLVVLYILLVEWAQDAKAAGTADMFNTLKLLTVVMWLGYPIVWALGVEGIAVLPVGVTSWGYSFLDIVAKYIFAFLLLNYLTSNESVVSGSILDVPSASGTPADDAAAKSRITSEGEYIPLDQIDINVVSKGEELFTGVVPILVELDGDVNGHKFSVSGEGEGDATYGKLTLKFICTTGKLPVPWPTLVTTFGYGLQCFARYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSYQSALSKDPNEKRDHMVLLEFVTAAGITLGMDELYKFCYENEV

SEQ ID NO: 4 - protein, 223 aa, Artificial sequence
  Synthetic amino acid sequence
ASSFGKALLEFVFIVFACITLLLGINAAKSKAASRVLFPATFVTGIASIAYFSMASGGGWVIAPDCRQLFVARYLDWLITTPLLLIDLGLVAGVSRWDIMALCLSDVLMIATGAFGSLTVGNVKWVWWFFGMCWFLHIIFALGKSWAEAAKAKGGDSASVYSKIAGITVITWFCYPVVWVFAEGFGNFSVTFEVLIYGVLDVISKAVFGLILMSGAATGYESI

SEQ ID NO: 5 - protein, 310 aa, Artificial sequence
  Synthetic amino acid sequence
MDYGGALSAVGRELLFVTNPVVVNGSVLVPEDQCYCAGWIESRGTNGAQTASNVLQWLAAGFSILLLMFYAYQTWKSTCGWEEIYVCAIEMVKVILEFFFEFKNPSMLYLATGHRVQWLRYAEWLLTCPVILIHLSNLTGLSNDYSRRTMGLLVSDIGTIVWGATSAMATGYVKVIFFCLGLCYGANTFFHAAKAYIEGYHTVPKGRCRQVVTGMAWLFFVSWGMFPILFILGPEGFGVLSVYGSTVGHTIIDLMSKNCWGLLGHYLRVLIHEHILIHGDIRKTTKLNIGGTEIEVETLVEDEAEAGAVP

SEQ ID NO: 6 - protein, 310 aa, Artificial sequence
  Synthetic amino acid sequence
MDYGGALSAVGRELLFVTNPVVVNGSVLVPEDQCYCAGWIESRGTNGAQTASNVLQWLAAGFSILLLMFYAYQTWKSTCGWEEIYVCAIEMVKVILEFFFEFKNPSMLYLATGHRVQWLRYAEWLLTSPVILIHLSNLTGLSNDYSRRTMGLLVSDIGTIVWGATSAMATGYVKVIFFCLGLCYGANTFFHAAKAYIEGYHTVPKGRCRQVVTGMAWLFFVSWGMFPILFILGPEGFGVLSVYGSTVGHTIIDLMSKNCWGLLGHYLRVLIHEHILIHGDIRKTTKLNIGGTEIEVETLVEDEAEAGAVP

SEQ ID NO: 7 - protein, 310 aa, Artificial sequence
  Synthetic amino acid sequence
MDYGGALSAVGRELLFVTNPVVVNGSVLVPEDQCYCAGWIESRGTNGAQTASNVLQWLAAGFSILLLMFYAYQTWKSTCGWEEIYVCAIEMVKVILEFFFEFKNPSMLYLATGHRVQWLRYAEWLLTSPVILIHLSNLTGLSNDYSRRTMGLLVSAIGTIVWGATSAMATGYVKVIFFCLGLCYGANTFFHAAKAYIEGYHTVPKGRCRQVVTGMAWLFFVSWGMFPILFILGPEGFGVLSVYGSTVGHTIIDLMSKNCWGLLGHYLRVLIHEHILIHGDIRKTTKLNIGGTEIEVETLVEDEAEAGAVP

SEQ ID NO: 8 - protein, 344 aa, Artificial sequence
  Synthetic amino acid sequence
MSRRPWLLALALAVALAAGSAGASTGSDATVPVATQDGPDYVFHRAHERMLFQTSYTLENNGSVICIPNNGQCFCLAWLKSNGTNAEKLAANILQWITFALSALCLMFYGYQTWKSTCGWEEIYVATIEMIKFIIEYFHEFDEPAVIYSSNGNKTVWLRYAEWLLTCPVLLIHLSNLTGLKDDYSKRTMGLLVSDVGCIVWGATSAMCTGWTKILFFLISLSYGMYTYFHAAKVYIEAFHTVPKGICRELVRVMAWTFFVAWGMFPVLFLLGTEGFGHISPYGSAIGHSILDLIAKNMWGVLGNYLRVKIHEHILLYGDIRKKQKITIAGQEMEVETLVAEEED

SEQ ID NO: 9 - protein, 344 aa, Artificial sequence
  Synthetic amino acid sequence
MSRRPWLLALALAVALAAGSAGASTGSDATVPVATQDGPDYVFHRAHERMLFQTSYTLENNGSVICIPNNGQCFCLAWLKSNGTNAEKLAANILQWITFALSALCLMFYGYQTWKSTCGWETIYVATIEMIKFIIEYFHEFDEPAVIYSSNGNKTVWLRYAEWLLTCPVLLIHLSNLTGLKDDYSKRTMGLLVSDVGCIVWGATSAMCTGWTKILFFLISLSYGMYTYFHAAKVYIEAFHTVPKGICRELVRVMAWTFFVAWGMFPVLFLLGTEGFGHISPYGSAIGHSILDLIAKNMWGVLGNYLRVKIHEHILLYGDIRKKQKITIAGQEMEVETLVAEEED

SEQ ID NO: 10 - protein, 344 aa, Artificial sequence
  Synthetic amino acid sequence
MSRRPWLLALALAVALAAGSAGASTGSDATVPVATQDGPDYVFHRAHERMLFQTSYTLENNGSVICIPNNGQCFCLAWLKSNGTNAEKLAANILQWITFALSALCLMFYGYQTWKSTCGWEEIYVATIEMIKFIIEYFHEFDEPAVIYSSNGNKTVWLRYATWLLTCPVLLIHLSNLTGLKDDYSKRTMGLLVSDVGCIVWGATSAMCTGWTKILFFLISLSYGMYTYFHAAKVYIEAFHTVPKGICRELVRVMAWTFFVAWGMFPVLFLLGTEGFGHISPYGSAIGHSILDLIAKNMWGVLGNYLRVKIHEHILLYGDIRKKQKITIAGQEMEVETLVAEEED

SEQ ID NO: 11 - protein, 344 aa, Artificial sequence
  Synthetic amino acid sequence
MSRRPWLLALALAVALAAGSAGASTGSDATVPVATQDGPDYVFHRAHERMLFQTSYTLENNGSVICIPNNGQCFCLAWLKSNGTNAEKLAANILQWITFALSALCLMFYGYQTWKSTCGWETIYVATIEMIKFIIEYFHEFDEPAVIYSSNGNKTVWLRYATWLLTCPVLLIHLSNLTGLKDDYSKRTMGLLVSDVGCIVWGATSAMCTGWTKILFFLISLSYGMYTYFHAAKVYIEAFHTVPKGICRELVRVMAWTFFVAWGMFPVLFLLGTEGFGHISPYGSAIGHSILDLIAKNMWGVLGNYLRVKIHEHILLYGDIRKKQKITIAGQEMEVETLVAEEED

SEQ ID NO: 12 - protein, 6 aa, Artificial sequence
  Synthetic amino acid sequence
FXYENE

SEQ ID NO: 13 - protein, 7 aa, Artificial sequence
  Synthetic amino acid sequence
FCYENEV

SEQ ID NO: 14 - protein, 5 aa, Artificial sequence
  Synthetic amino acid sequence
VKESL

SEQ ID NO: 15 - protein, 5 aa, Artificial sequence
  Synthetic amino acid sequence
VLGSL

SEQ ID NO: 16 - protein, 16 aa, Artificial sequence
  Synthetic amino acid sequence
NANSFCYENEVALTSK

SEQ ID NO: 17 - protein, 20 aa, Artificial sequence
  Synthetic amino acid sequence
KSRITSEGEYIPLDQIDINV

SEQ ID NO: 18 - protein, 18 aa, Artificial sequence
  Synthetic amino acid sequence
MTETLPPVTESAVALQAE

SEQ ID NO: 19 - protein, 26 aa, Artificial sequence
  Synthetic amino acid sequence
MDYGGALSAVGRELLFVTNPVVVNGS

SEQ ID NO: 20 - protein, 27 aa, Artificial sequence
  Synthetic amino acid sequence
MAGHSNSMALFSFSLLWLCSGVLGTEF

SEQ ID NO: 21 - protein, 23 aa, Artificial sequence
  Synthetic amino acid sequence
MGLRALMLWLLAAAGLVRESLQG

SEQ ID NO: 22 - protein, 18 aa, Artificial sequence
  Synthetic amino acid sequence
MRGTPLLLVVSLFSLLQD